(19) United States
(12) Patent Application Publication
(10) Pub. No.: US 2012/0190012 A1
(43) Pub. Date: Jul. 26, 2012

(54) COMPOSITIONS AND METHODS FOR DNA SEQUENCING

(75) Inventors: Nathaniel G. BUTLIN, Pacifica, CA (US); Ga-Lai LAW, Berekeley, CA (US); Christopher ANDOLINA, Berkeley, CA (US)

(73) Assignee: LUMIPHORE, INC., Richmond, CA (US)

(21) Appl. No.: 13/081,407

(22) Filed: Apr. 6, 2011

Related U.S. Application Data
(60) Provisional application No. 61/321,328, filed on Apr. 6, 2010.

(51) Int. Cl. G01N 21/64; C07C 237/30; C07D 487/18

(52) U.S. Cl. 435/6.1; 540/461; 564/165

(57) ABSTRACT

The invention provides compositions and methods useful in DNA sequencing. In exemplary embodiments, a detectable label such as a luminescent macrocycle is used.

### CROSS-REFERENCE TO RELATED APPLICATIONS

This application claims under 35 USC 119(e) the benefit of U.S. Patent Application 61/321,328, filed Apr. 6, 2010, which is incorporated by reference in its entirety.

### TECHNICAL FIELD

The field of the invention relates to nucleic acid detection.

### BACKGROUND

There is a continuous and expanding need for rapid, highly specific methods of detecting and quantifying chemical, biochemical and biological substances as analytes in research and diagnostic mixtures. Of particular value are methods for measuring small quantities of nucleic acids, peptides, pharmaceuticals, metabolites, microorganisms and other materials of diagnostic value. Examples of such materials include small molecular bioactive materials (e.g., narcotics and poisons, drugs administered for therapeutic purposes, hormones), pathogenic microorganisms and viruses, antibodies, and enzymes and nucleic acids, particularly those implicated in disease states.

The presence of a particular analyte can often be determined by binding methods that exploit the high degree of specificity, which characterizes many biochemical and biological systems. Frequently used methods are based on, for example, antigen-antibody systems, nucleic acid hybridization techniques, and protein-ligand systems. In these methods, the existence of a complex of diagnostic value is typically indicated by the presence or absence of an observable “label” which is attached to one or more of the interacting materials. The specific labeling method chosen often dictates the usefulness and versatility of a particular system for detecting an analyte of interest. Preferred labels are inexpensive, safe, and capable of being attached efficiently to a wide variety of chemical, biochemical, and biological materials without significantly altering the important binding characteristics of those materials.

A wide variety of labels are known, each with particular advantages and disadvantages. For example, radioactive labels are quite versatile, and can be detected at very low concentrations. Such labels are, however, expensive, hazardous, and their use requires sophisticated equipment and trained personnel. Thus, there is wide interest in non-radioactive labels, particularly labels observable by spectrophotometric, spin resonance, and luminescence techniques, and reactive materials, such as enzymes that produce such molecules.

Labels that are detectable using fluorescence spectroscopy are of particular interest, because of the large number of such labels that are known in the art. Moreover, the literature is replete with syntheses of fluorescent labels that are derivatized to allow their facile attachment to other molecules, and many such fluorescent labels are commercially available.

In addition to being directly detected, many fluorescent labels operate to quench the fluorescence of an adjacent second fluorescent label. Because of its dependence on the distance and the magnitude of the interaction between the quencher and the fluorophore, the quenching of a fluorescent species provides a sensitive probe of molecular conformation and binding, or other, interactions. An excellent example of the use of fluorescent reporter quencher pairs is found in the detection and analysis of nucleic acids.

Conventional organic fluorophores generally have short fluorescence lifetimes, on the order of nanoseconds (ns) which is generally too short for optimal discrimination from background fluorescence. An alternative detection scheme, which is theoretically more sensitive than conventional fluorescence, is time-resolved fluorimetry. According to this method, a chelated lanthanide metal with a long radiative lifetime is attached to a molecule of interest. Pulsed excitation combined with a gated detection system allows for effective discrimination against short-lived background emission. For example, using this approach, the detection and quantification of DNA hybrids via an europium-labeled antibody has been demonstrated (Syvanen et al., Nucleic Acids Research 14: 1017 1028 (1986)). In addition, biotinylated DNA was measured in microtiter wells using Eu-labeled strepavidin (Dahlen, Anal. Biochem. (1982), 164: 78 83). A disadvantage, however, of these types of assays is that the label must be washed from the probe and its fluorescence developed in an enhancement solution.

Lanthanide chelates, particularly coordinatively saturated chelates that exhibit excellent fluorescence properties are highly desirable. Alternatively, coordinatively unsaturated lanthanide chelates exhibiting acceptable fluorescence in the presence of water are also advantageous. Such chelates that are derivatized to allow their conjugation to one or more components of an assay, find use in a range of different assay formats. The present invention provides these and other such compounds and assays using these compounds. Complexes of lanthanide ions such as Tb³⁺ and Eu³⁺ are potentially useful in a variety of biological applications. Of particular importance for biological applications is that these complexes exhibit kinetic stability at high dilution in aqueous solutions, i.e., concentrations at or below nM levels.

Hydroxyisophthalamide ligands useful in applications requiring luminescence have been described (Petoud et al., J. Am. Chem. Soc. 2003, 125, 13324-13325; U.S. Pat. No. 7,018,850 to Raymond et al.), and Johansson et al., J. Am. Chem. Soc. 2004, 126(50):16451-16455).

However, a need for luminescent complexes, which are stable under biological relevant conditions and at low concentrations, and which simultaneously exhibit low non-specific interactions with proteins, remains. Moreover, multiplex assays in which more than one fluorophore undergoes excitation and detection are of use in many fields. Thus, there is a continuing need for fluorescent systems amenable to incorporation in such multiplex assays. The current invention addresses these and other needs.

### SUMMARY OF INVENTION

The invention provides a new class of macrocyclic ligands and metal complexes of these ligands. Also provided are conjugates of these ligands with carrier moieties, which are of use in single fluorophore and multiplex applications. The invention also provides mixtures of carrier moieties, each conjugated to a chelate of the invention. Moreover, there are provided mixtures of carrier moieties in which one or more of a first carrier moiety species is conjugated to a chelate of the invention and one or more of a second carrier moiety species is conjugated to a fluorophore different in structure from the chelate attached to the first carrier moiety species. The invention also provides single fluorophore and multiplex assays incorporating one or more chelates of the invention. It is generally preferred that the chelates be bound to a metal ion, which, together with the chelate, forms a luminescent metal ion complex.

In particular, the invention provides methods of using macrocyclic ligands and metal complexes of these ligands. In one aspect, the invention provides a method of detecting a nucleotide, the method comprising: (a) forming a complex between the nucleic acid and a DNA polymerase comprising a first luminescent group, wherein the nucleic acid comprises a template strand and a primer hybridized to the template strand; (b) extending the primer with a dNTP comprising a second luminescent group by contacting the complex with the dNTP; (c) exciting the complex with light, whereby energy is transferred between the first luminescent group and the second luminescent group; and (d) detecting energy emitted by the complex, thereby detecting the nucleotide.

In one aspect, the invention provides a method of detecting a nucleotide, the method comprising: (a) forming a complex between a nucleic acid and a capture probe, wherein the capture probe is bound to a solid support; (b) contacting the complex with a DNA polymerase and a blocked dNTP comprising a first luminescent group and a second luminescent group, thereby extending the nucleic acid with the blocked dNTP; (c) washing the solid support; (d) exciting the complex with light, whereby energy is transferred between the first luminescent group and the second luminescent group; and (e) detecting energy emitted by the second luminescent group, thereby detecting the nucleotide. The methods disclosed herein can be practiced using the various compounds, such as luminescent groups, energy transfer donors and energy transfer acceptors, disclosed herein.

The invention also provides luminescent complexes, e.g., lanthanide (e.g., terbium and europium) complexes and conjugates of these complexes with a carrier moiety. These complexes exhibit high stability and solubility in aqueous media as well as high quantum yields of luminescence in water without external augmentation (e.g., by micelles or fluoride). The complexes are formed between a metal ion, e.g., of the lanthanide series and a new class of macrocyclic ligands provided by the invention. Preferred ligands incorporate a hydroxy-containing aromatic building block, such as a 2-hydroxy-1,3-amine or -amide (e.g., hydroxy-isophthalamide) moieties within their structure and are characterized by surprisingly low non-specific binding to a variety of different polypeptides such as antibodies and proteins. Because of their unique chemical and physicochemical properties, the complexes of the present invention find use in any application requiring luminescence, particularly in aqueous media, including medical diagnostics and bioanalyical assay systems.

In a first embodiment, the invention provides a compound that includes a chelate structure according to Formula I:

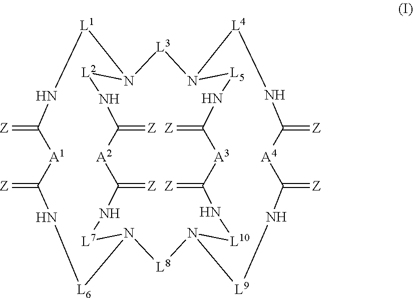

wherein the compound preferably includes at least one acceptor-linker, which is optionally covalently bound to a fluorophore, e.g., through a linkage fragment. In another embodiment, the compound of the invention includes at least one functional moiety. In a still further embodiment, a reactive functional group on the functional moiety is converted to a linkage fragment by reaction with a complementary reactive group on a carrier moiety, e.g., a nucleic acid, a peptide, an antibody, a saccharide, lectin, receptor or antigen, or a solid support.

In Formula I, each Z is a member independently selected from O and S. L¹, L², L³, L⁴, L⁵, L⁶, L⁷, L⁸, L⁹ and L¹⁰ (“L^{x}” moieties) are linker groups independently selected from substituted or unsubstituted alkyl, substituted or unsubstituted heteroalkyl, substituted or unsubstituted aryl, substituted or unsubstituted heteroaryl and substituted or unsubstituted heterocycloalkyl.

A¹, A², A³ and A⁴ are members independently selected from substituted or unsubstituted aryl and substituted and unsubstituted heteroaryl (e.g., an azulene group) moieties. In an exemplary embodiment, these moieties are independently selected from the following structure:

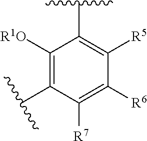

wherein each R¹ is a member independently selected from H, an enzymatically cleavable group, a hydrolytically cleavable group, a metabolically cleavable group and a single negative charge. Each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —C(O)R¹⁸ —COOR¹⁷, —CONR¹⁷R¹⁸, —S(O)₂OR¹⁷, —OC(O)R¹⁷, —C(O)NR¹⁷R¹⁸, —NR¹⁷C(O)R¹⁸, —NR¹⁷SO₂R¹⁸, and —NO₂, wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl.

R¹⁷ and R¹⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R¹⁷ and R¹⁸, together with the atoms to which they are attached, are optionally joined to form a 5- to 7-membered ring.

In an exemplary embodiment, a compound according to Formula I is covalently attached through the functional moiety to a carrier molecule.

Exemplary compounds according to any of the embodiments discussed above, are those in which at least one L^{x} moiety is functionalized with a acceptor-linker, optionally bound to a fluorophore, e.g., through a linkage fragment. In another embodiment, at least one of L^{x} moiety is functionalized with a functional moiety (optionally bound to a carrier moiety or solid support, e.g., through a linkage fragment). In yet another exemplary embodiment, one of these L^{x} groups is functionalized with a acceptor-linker (optionally bound to a fluorophore) and the same or a different L^{x} moiety is functionalized with a functional moiety (optionally bound to a carrier moiety or solid support). As will be appreciated by those of skill in the art, a fluorophore coupled to a acceptor-linker can be an organic fluorophore or a macrocyclic chelate, e.g., such as the structure set forth in Formula I. Moreover, a branched fluorophore with more than one reactive functional group can be use to couple more than one fluorophore through the reactive functional groups, whether the fluorophore is a wholly organic species or is a metal chelate.

In one aspect, the invention provides a luminescent complex formed between at least one metal ion and a chelate according to Formula I, described below. In contrast to organic fluorophores that have a fluorescence lifetime of about 10 ns, lanthanide chelates of the invention preferably have emission lifetimes greater than 100 microseconds, preferably at least 500 microseconds and even more preferably at least 1 ms. The mechanism that is responsible for the long lifetime emission of lanthanide chelates involves energy transfer from the triplet state of the aromatic ligand. Specifically, upon excitation the ligand is excited to its singlet state and then undergoes an intersystem transition to its triplet state, transferring the energy to the lanthanide ion. Fluorescence is then emitted from the lanthanide ion as it returns to the ground state. Since such fluorescence emission does not result from a singlet-to-singlet transition, the use of lanthanide chelates as a donor results in luminescent resonance energy transfer (LRET). Therefore, by using pulse excitation and time-gating techniques, emission from the fluorophore can be selectively recorded after the background fluorescence from organic dyes, scattering, and autofluorescence has decayed. The only signals remaining in this long-time domain are the emission from the lanthanide chelate and from acceptor fluorophores that have participated in LRET. In this case the narrow emission peaks of a lanthanide chelate render the background fluorescence close to zero at certain wavelengths, leading to extremely large signal-to-background ratio.

In one aspect, the invention provides a compound according to Formula I in a mixture with an analyte. Exemplary analytes include nucleic acids, peptides, antibodies, antigens, lectins, saccharides, cells and receptors.

In one aspect, the invention provides a method of detecting an analyte in a sample, said method comprising: (a) contacting said analyte with a solid support comprising a luminescent complex of the invention, wherein said analyte forms an analyte complex; (b) exciting said luminescent complex such that said luminescent complex transfers excitation energy to said analyte complex; and (c) detecting energy emitted by said analyte complex, thereby detecting said analyte.

In yet a further example, the luminescence modifying group and/or the fluorophore and/or the complex of the invention is a component covalently bound to the analyte.

In one aspect, the invention provides a compound having the structure: Q¹-G wherein Q¹ is a luminescent group and G is a cleavable group.

In one aspect, the invention provides a compound having the structure: Q²-G wherein Q² comprises an energy transfer donor and an energy transfer acceptor, G is a cleavable group.

### BRIEF DESCRIPTION OF THE DRAWINGS

FIG. 1 Four Color FRET Transfer Dyes. A. Covalent synthetic coupling of 4-Tb with a conventional dye via a variable length linker. Various synthetic methods could allow a remaining reactive linker anchored to the 4-Tb (R1), the acceptor-linker (R2), or the Acceptor Dye (R3). B. A series of acceptor dyes are joined to the 4-Tb donor and are tuned to each of the different emission peaks.

FIG. 2 Absorption and emission spectra of 4-Tb fluorophore. The four emission peaks (A, B, C, and D) characteristic of terbium offer the potential for multicolor emission based on fluorescent resonance energy transfer.

FIG. 3 Structures of exemplary acceptors for the four major 4-Tb Donor peaks. A) Structure of 5/6 Carboxy Fluorscein (FAM) with 494 nm excitation by Tb Donor Peak at 492 nm and a 518 nm emission. B) 5/6 tetramethylrhodamine Isothiocyanate (TRITC) with 544 nm excitation by Tb Donor Peak at 545 nm and a 572 nm emission. C) Texas Red Sulfonyl Chloride with 588 nm excitation by Tb Donor Peak at 590 nm and a 612 nm emission. D) Cy5 with 649 nm excitation by Tb Donor Peak at 620 nm and a 670 nm emission.

FIG. 4 Chemical model of the isophthalamide based macrocyclic ligand Lumi4®-Tb. Structure is shown with generic linker arm that has been modified for varied reactivites. The chelate binds trivalent lanthanide ions and transfers energy efficiently to a coordinated terbium. The Lumi-4-Tb complex shown is thermodynamically stable and kinetically inert. “R” is any appropriate “R” moiety as disclosed herein.

FIG. 5 Time resolved fluorescence measurements remove interfering short-lived auto fluorescence of biological samples and containers, increasing sensitivity and signal/noise ratios.

FIG. 6 Ligand 1, an octadentate acyclic 2-hydroxyiosphthalamide (A-IAM) ligand efficiently sensitizes trivalent lanthanide ions such as Tb³⁺. Ligand 2, a macrocyclic version (M-IAM) of this complex, exhibits much improved kinetic stability of the metal complex, making it more suitable for biological applications. The commercialized version Lumi4 includes a functionalized linker for biomolecule coupling.

FIG. 7 Lanthanide luminescence stability of dilute solutions (5 nM) of Tb complexes of ligands 1, 2 and 3 (FIG. 6) as measured in various aqueous solutions at room temperature as a function of time. All solutions contained 0.05% Tween-20. All of the complexes are stable in neutral buffers. While the macrocyclic complex Tb-2 shows much improved kinetic stability over its acyclic sibling Tb-1, the derivatized macrocycle, Tb-3, exhibits exceptional kinetic stability even in the presence of EDTA and 1% acetic acid, making it attractive for a variety of applications in biotechnology. Reagents incorporating Tb-3 or a related amine derivative retain their luminescent properties formulated at nanomolar concentrations for greater than 12 months when stored at 4° C.

FIG. 8 (A) Exponential decay lifetime comparison between Lumi-4-Tb (2.69 msec) and LanthaScreen™-Tb (1.67 msec). Lumiphore's complex exhibits a longer decay lifetime. (B) The steady state spectra of bioconjugates of streptavidin (SA), bovine gamma globulin (BGG), and a monoclonal mouse antibody overlap almost identically. (C) Relative time-resolved fluorescence of Lumiphore and Invitrogen's streptavidin-Tb conjugates shows that Lumiphore's conjugates are more than an order of magnitude brighter, which translates to greater signal at lower concentrations. (D) Direct comparisons of Lumiphore and Invitrogen's streptavidin-Tb conjugates in an exemplary FRET assay using biotinylated fluorescein demonstrates that Lumiphore's conjugate generates equivalent signal when using 10× less streptavidin-Tb and biotinylated fluorescein. Both conjugates had ˜4.4 metal complexes per streptavidin and all dilutions were performed in Tris-buffered saline, pH 7.6+0.05% Tween-20. Lifetime data was recorded in 0.1 M Tris, pH 7.4.

FIG. 9 Absorption and emission spectra of Lumi-4-Tb fluorophore. The four emission peaks (A, B, C, and D) characteristic of Terbium offer the potential for multicolor emission based on fluorescent resonance energy transfer.

FIG. 10 Dye conjugate experimental photophysical property comparison.

FIG. 11 Synthetic constructs and accompanying spectra. Absorbance (blue) indicates expected peaks at 340 nm (Lumi4-Tb) and acceptor specific wavelengths. Conventional acceptor emission peak is indicated (grey) overlapping the time resolved spectra (red). Time resolved spectra show some characteristics of the donor dye from less than 100% efficient energy transfer.

FIG. 12 Normalized absorbance (solid) and emission (dashed) spectra of TBFL (Lumi4-Fluorescein) construct before (blue) and after (red) conjugation to streptavidin. Essentially identical absorption and emission peak shapes and maxima indicate no perturbation of the novel fluorophores photophysical properties following conjugation to protein. Measurements were taken in biologically relevant buffer at near neutral pH.

FIG. 13 The structure of 2-nitrobenzyl and the photo-cleaving reaction. Following UV irradiation, the nitrobenzyl moiety leaves a 3′ hydroxyl group available for base incorporation in the developing strand.

FIG. 14 Structure showing positions for modification and attachment of the dye.

FIG. 15 General design of reaction flow chamber.

FIG. 16 Chips spotted with specific probe sequences are incubated with sample DNA segments. Following compliment annealing, a ligase wash/incubation results in covalent attachment of target sequence to chip surface. Each spot will consists of 100's of unique probe sequences.

FIG. 17 Rapid sequencing occurs in cycles consisting of four steps. (A) dNTP incorporation reagents are flushed over the low volume chip, (B) DNA polymerase incorporates a single nucleotide with time resolved fluorescent tag at a blocking location, (C) reagents and polymerase are rigorously washed from chip surface and, (D) excitation at 340 nm reveals a base specific signal against zero background with rapid cleavage of blocking fluorophore. The growing strand is ready to begin a second cycle.

FIG. 18 Alternate sequencing approach leveraging exceptional FRET efficencies. Single immobilized polymerases process target strand DNA incorporating blocked dNTP's (photocleavage with excitation source unblocks 3′ hydroxyl position) or is allowed to process in real-time. As new base enters the binding pocket only then are distances small enough to allow FRET transfer to occur yielding a signal of wavelength specific to the nucleotide. Continuous irradiation and CCD image capture can follow real-time polymerase action.

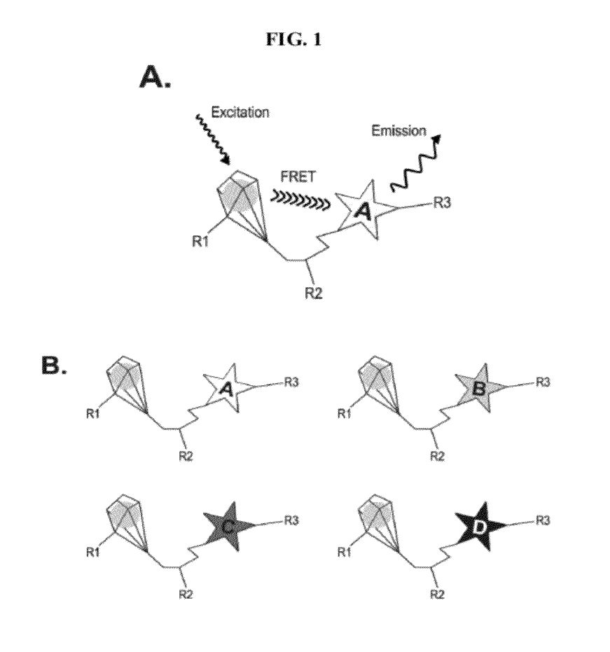

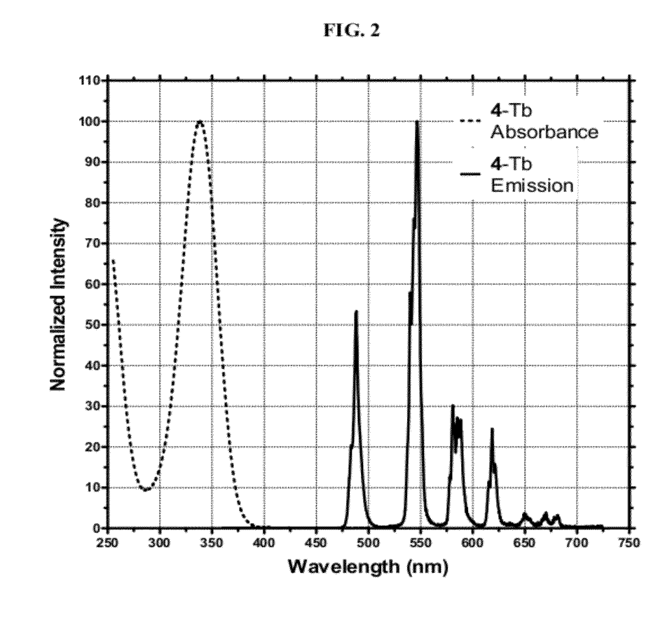

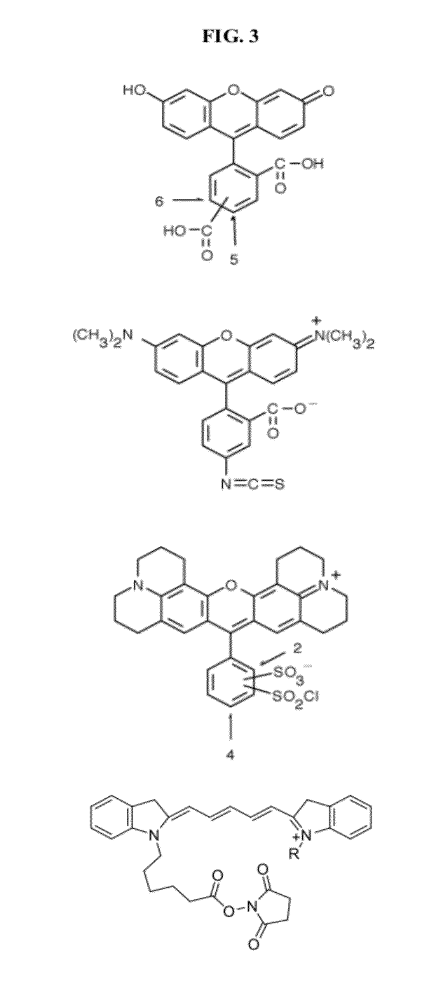

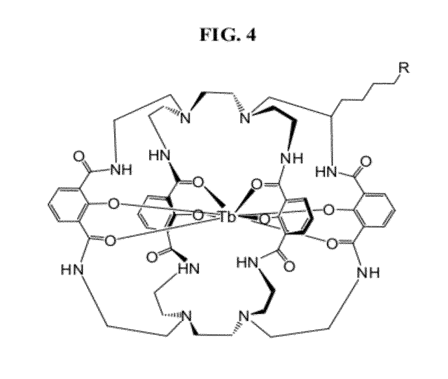

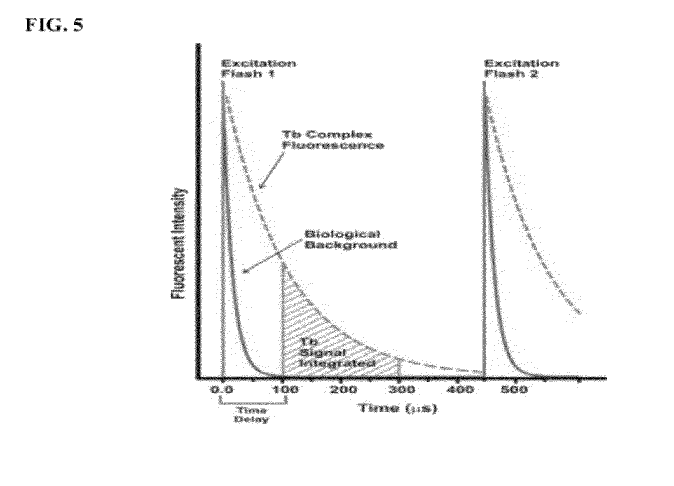

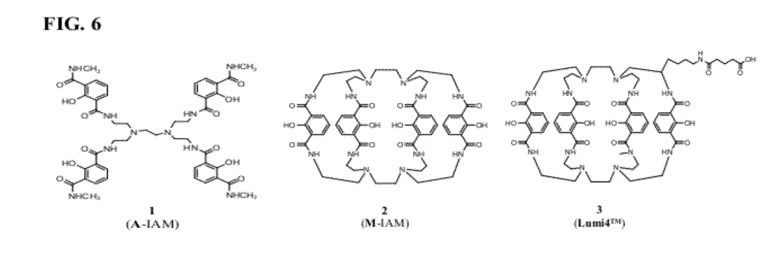

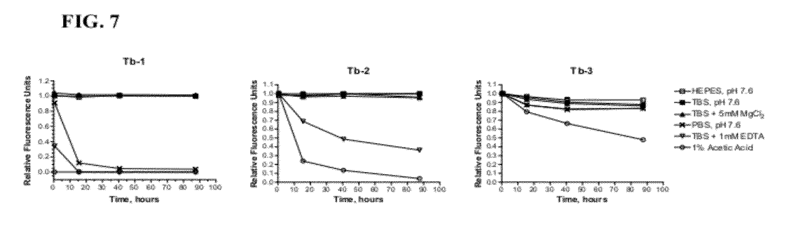

### DESCRIPTION OF EMBODIMENTS

### Definitions

“Analyte”, as used herein, means any compound or molecule of interest for which a diagnostic test is performed, such as a biopolymer or a small molecular bioactive material. An analyte can be, for example, a cell, a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, nucleic acid, lipid, without limitation. An analyte can have bound thereto a fluorophore as defined herein and/or a compound according to Formula I. An analyte can be bound to a carrier moiety or to a solid support.

As used herein, “energy transfer” refers to the process by which energy emission of an excited donor (e.g., a luminescent group) is altered by an acceptor (e.g., a luminescence-modifying group). When the luminescence-modifying group is a quenching group then the energy emission from the luminescent group is attenuated (quenched). Energy transfer mechanisms include luminescence resonance energy transfer, e.g., by dipole-dipole interaction (e.g., in longer range energy transfer) or electron transfer (e.g., across shorter distances). An exemplary mechanism involves transfer of energy from a metal chelate to a fluorophore (or a quencher or other luminescence modifying group) covalently bound to the chelating moiety through a linker, such as the compounds of the invention described herein. While energy transfer is often based on spectral overlap of the emission spectrum of the luminescent group and the absorption spectrum of the luminescence-modifying group, (in addition to distance between the groups) it has been demonstrated that spectral overlap is not necessarily required for energy transfer to occur (see, e.g., Latva et al., U.S. Pat. No. 5,998,146, which is incorporated herein by reference) and this type of energy transfer is encompassed within the present invention. Energy transfer between members of an energy transfer pair occurs when the members of the pair are in “operative proximity,” which is defined herein as a distance between the members of the pair that allows detectable energy transfer to occur. It is to be understood that any reference to “energy transfer” in the instant application encompasses all mechanistically-similar phenomena.

“Energy transfer pair” is used to refer to a group of molecules that participate in energy transfer. Such complexes may comprise, for example, two luminescent groups, which may be different from one-another and one quenching group, two quenching groups and one luminescent group, or multiple luminescent groups and multiple quenching groups. In cases where there are multiple luminescent groups and/or multiple quenching groups, the individual groups may be different from one another. Typically, one of the molecules acts as a luminescent group, and another acts as a luminescence-modifying group. The preferred energy transfer pair of the invention comprises a luminescent group of the invention and a fluorophore (e.g., an organic fluorophore). The fluorophore can act as a quencher or other luminescence modifying group or, rather than a fluorophore, the acceptor-linker can be conjugated to a quencher or other luminescence modifying moiety. There is no limitation on the identity of the individual members of the energy transfer pair in this application. Generally preferred energy transfer pairs are characterized by a change in the spectroscopic properties of the pair if the distance between the individual members is altered by some critical amount. An exemplary energy transfer pair is a luminescent complex of the invention and an organic fluorophore.

As used herein, “luminescence-modifying group” refers to a molecule of the invention that can alter in any way the luminescence emission from a luminescent group. A luminescence-modifying group generally accomplishes this through an energy transfer mechanism. Depending on the identity of the luminescence-modifying group, the luminescence emission can undergo a number of alterations, including, but not limited to, attenuation, complete quenching, enhancement, a shift in wavelength, a shift in polarity, and a change in luminescence lifetime. One example of a luminescence-modifying group is a fluorophore that participates with a metal complex component of a complex of the invention in fluorescence resonance energy transfer. Another exemplary luminescence-modifying group is a quenching group.

As used herein, “quenching group” refers to any luminescence-modifying group of the invention that can attenuate at least partly the light emitted by a luminescent group. This attenuation is referred to herein as “quenching”. Hence, excitation of the luminescent group in the presence of the quenching group leads to an emission signal that is less intense than expected, or even completely absent. Quenching typically occurs through energy transfer between the luminescent group and the quenching group.

“Fluorescence resonance energy transfer” or “FRET” is used interchangeably with and “LRET” and refers to an energy transfer phenomenon in which the excited state energy (e.g., light) emitted by an excited luminescent group is absorbed at least partially by a luminescence-modifying group of the invention and re-emitted at a different (e.g., longer) wavelength by the luminescence-modifying group. FRET depends on energy transfer between the luminescent group and the luminescence-modifying group. The efficiency of FRET depends at least in part on the distance between the luminescence modifying group and the luminescent group. In contrast to excimers and exciplex fluorescence, FRET pairs do not require the dye molecules forming the complexes to be in very close proximity. FRET is commonly used in several detection modes to detect, characterize or identify a variety of biologically active molecules including nucleic acids, e.g., oligonucleotides, peptides (e.g., peptides including one or more protease cleaveage site) and proteins (e.g., antibodies, antigens, receptors). One of the advantages of FRET is that fluorescence arises under physiologically relevant conditions (e.g., pH between about 7 and about 8, e.g., 7.3-7.5) in comparison to exciplex fluorescence which is typically weak under aqueous conditions, requiring the addition of organic solvents or formation in a similar molecular microenvironment. In an one embodiment, the compound according to Formula I is incorporated into a nucleic acid having a motif of a known dual- or multiple-labeled nucleic acid probe (e.g., Molecular Beacons, Scorpion probes, TaqMan, and the like). The compound according to Formula I and the fluorophore can be positioned analogously to the donor and acceptor moieties of such probes.

“Moiety” refers to the radical of a molecule that is attached to another moiety.

The term “targeting moiety” is intended to mean any moiety conjugated to the complexes of the invention that targets the complex to a selected target (e.g., a complementary nucleic acid, a receptor structure, an antibody, an antigen, a lectin). The targeting moiety can be a small molecule (e.g., MW<500D), which is intended to include both non-peptides and peptides. The targeting group can also be a macromolecule, which includes saccharides, lectins, receptors, ligands for receptors, proteins such as BSA, antibodies, nucleic acids, solid supports and so forth. The targeting moiety can be a component of the complex of the invention. For example, in one embodiment, the targeting moiety is the acceptor-linker (e.g., the acceptor-linker is a nucleic acid with a sequence sufficiently complementary to the target to allow hybridization between the acceptor-linker and the target). In another embodiment, the targeting moiety is a group conjugated to the functional moiety (e.g., a nucleic acid, antibody, antigen, biotin, avidin, streptavidin, etc.).

“Carrier moiety” or “carrier” as used herein refers to a species to which a compound according to Formula I is covalently bound through reaction of a reactive functional group on a functional moiety with a reactive functional group of complementary reactivity on the carrier moiety. Exemplary carrier moieties include nucleic acids (DNA, RNA), peptides, antibodies (such as IgG), antibody fragments, antigens, receptors, lectins, saccharides, lipids and the like. Further exemplary carriers include biotin, avidin and streptavidin. A “carrier moiety” can function as a “targeting moiety.”

The term, “fluorophore,” as used herein refers to a species of excited energy acceptors capable of generating fluorescence when excited, which has a structure other than that shown in Formula I or a luminescent metal complex of Formula I. Complexes of different metal ions incorporating the structure according to Formula I are considered to be different compounds. Thus, for example, if a Tb chelate is a complex according to Formula I, an identical Eu complex can be a “fluorophore” according to the present invention. A fluorophore can be covalently bound to a compound according to Formula I through a acceptor-linker. Alternatively, the fluorophore can be bound to a first component of an assay, and the compound according to Formula I bound to a second component of an assay. Generally, it is preferred that the fluorophore is bound to the first assay component at a position and in a manner that allows energy transfer between the compound according to Formula I and the fluorophore when the first and second assay components interact in the assay. An exemplary assay is a hybridization assay in which a fluorophore is bound to a first nucleic acid and a compound according to Formula I is bound to a second nucleic acid. Other exemplary acceptors include quenchers and luminescence modifying moieties.

As used herein, “linker”, “linker moiety”, “linker group” or “linkage” (used interchangeably) refers to a moiety that joins a first portion of a compound to a second portion of the compound. For example, a linker or a linkage may join the chelating moiety of a compound of the invention to another species (e.g., carrier moiety or solid support). Exemplary linkers join a reactive functional group (“functional moiety”) or a fluorophore (“acceptor-linker”) to the chelating moiety of a compound of the invention. A linker can be any useful structure including, but not limited to 0-order linkers (i.e., a bond), acyl, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, a linker or a linkage is a bond. Further exemplary linkers include substituted or unsubstituted branched or linear C₁-C₁₀ substituted or unsubstituted alkyl and substituted or unsubstituted heteroalkyl. Other linkers include nucleic acids and peptides, such as PCR probes, hybridization probes and peptides that include protease cleaveage sites. Still further linkers include antibodies, lectins, haptens and saccharides.

As used herein, “nucleic acid” means DNA, RNA, single-stranded, double-stranded, or more highly aggregated hybridization motifs, and any chemical modifications thereof. Exemplary modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Modifications can also include 3′ and 5′ modifications such as capping with a quencher, a fluorophore, an intercalator, a minor groove binder or another moiety. Exemplary nucleic acids will bind, preferably under stringent conditions, to a nucleic acid of diagnostic interest. Preferred nucleic acids of diagnostic interest are those that are correlated with a disease, condition or syndrome, or progression, amelioration or treatment of a disease, condition or syndrome. Nonlimiting examples of nucleic acids include those that are sufficiently complementary, to bind under stringent conditions, to a nucleic acid from hepatitis (e.g., A, B or C), human papilloma virus (HPV), human immunodeficiency virus (HIV), influenza, Severe Acute Respiratory Syndrome Virus (SARS), gram positive and gram negative bacteria, and antibiotic resistant bacterial infections, e.g., multiple resistant Staphylococcus (MRS).

“Peptide” refers to a homo- or hetero-polymer or -oligomer in which the monomers are amino acids and are joined together through amide bonds, alternatively referred to as a polypeptide. When the amino acids are alpha-amino acids, either the L-optical isomer or the D-optical isomer can be used. Additionally, unnatural amino acids, for example, beta.-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. The term “peptide” or “polypeptide”, as used herein, refers to naturally occurring as well as synthetic peptides. In addition, peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

“Reactive functional group,” as used herein, has the meaning generally recognized in the art of synthetic chemistry, particularly bioconjugate chemistry. Exemplary reactive functional groups included, without limitation, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Methods to prepare each of these functional groups are well-known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

Where substituent groups are specified by their conventional chemical formulae, written from left to right, they equally encompass the chemically identical substituents, which would result from writing the structure from right to left, e.g., —CH₂O— is intended to also recite —OCH₂—. Thus, the incorporation of a moiety depicted with two attachment points into a larger structure is not limited to the depicted orientation of the moiety.

The term “alkyl,” by itself or as part of another substituent, means, unless otherwise stated, a straight or branched chain, or cyclic hydrocarbon radical, or combination thereof, which may be fully saturated, mono- or polyunsaturated and includes mono-, di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds (i.e., alkenyl and alkynyl moieties). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. Typically, an alkyl (or alkylene) group will have from 1 to 30 carbon atoms. A “lower alkyl” or “lower alkylene” is a shorter chain alkyl or alkylene group, generally having eight or fewer carbon atoms. In some embodiments, alkyl refers to an alkyl or combination of alkyls selected from C₁, C₂, C₃, C₄, C₅, C₆, C₇, Cₛ, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉ and C₃₀ alkyl. In some embodiments, alkyl refers to C₁-C₂₅ alkyl. In some embodiments, alkyl refers to Cᵣ C₂₀ alkyl. In some embodiments, alkyl refers to C₁-C₁₅ alkyl. In some embodiments, alkyl refers to C₁-C₁₀ alkyl. In some embodiments, alkyl refers to C₁-C₆ alkyl.

The terms “alkoxy,” “alkylamino” and “alkylthio” (or thioalkoxy) are used in their conventional sense, and refer to those alkyl and heteroalkyl groups attached to the remainder of the molecule via an oxygen atom, a nitrogen atom (e.g., an amine group), or a sulfur atom, respectively.

The term “heteroalkyl,” by itself or in combination with another term, means, unless otherwise stated, a stable straight or branched chain, or cyclic alkyl moiety, or combinations thereof, consisting of a number (e.g., a stated number) of carbon atoms and at least one heteroatom selected from the group consisting of O, N, Si, B and S, and wherein the nitrogen and sulfur atoms may optionally be oxidized and the nitrogen heteroatom may optionally be quaternized. The heteroatom(s) O, N, S, B and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′ C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “acyl” refers to a species that includes the moiety —C(O)R, where R has the meaning defined herein.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

In some embodiments, any of alkyl, heteroalkyl, cycloalkyl, heterocycloalkyl, aryl and heteroaryl is optionally substituted. That is, in some embodiments, any of these groups is substituted or unsubstituted. In some embodiments, substituents for each type of radical are selected from those provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted heteroalkyl, substituted or unsubstituted heterocycloalkyl, substituted or unsubstituted aryl, substituted or unsubstituted heteroaryl, halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′—or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), silicon (Si) and boron (B).

A “linkage fragment” is formed by reaction of a reactive functional group on one species with reactive functional group of complementary reactivity on another species (e.g., a fluorophore and an acceptor-linker, a functional moiety and a carrier moiety (or solid support). Exemplary linkage fragments formed by such reactions include, but are not limited to S, SC(O)NH, SC(O)(NH)₂, HNC(O)S, SC(O)O, O, NH, NHC(O), (NH)₂C(O), (O)CNH and NHC(O)O, and OC(O)NH, CH₂S, CH₂O, CH₂CH₂O, CH₂CH₂S, (CH₂)ₚO, (CH₂)ₚS or (CH₂)ₚY′-PEG wherein, Y′ is S, NH, NHC(O), C(O)NH, NHC(O)O, OC(O)NH, or O and p is an integer from 1 to 50.

The symbol “R” is a general abbreviation that represents a substituent group that is selected from acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

The term “cleavable group” refers to a group that is susceptible to cleavage under appropriate conditions. In exemplary embodiments, a cleavable group is selected from a hydrolytically cleavable group, an enzymatically cleavable group and a photolytically cleavable group. An “enzymatically cleavable” group refers to a group that is susceptible to being cleaved by an enzyme. In some embodiments, an enzymatically cleavable group is a substrate to an enzyme that is capable of cleaving the substrate. Numerous substrate/enzyme pairs are known in the art. A “photolytically cleavable” group refers to a group that is susceptible to being cleaved upon exposure to electromagnetic radiation, such as light. A “hydrolytically cleavable” group refers to a group that is susceptible to be cleaved by hydrolysis as is understood in the art.

The present invention includes all salt forms of those molecules that contain ionizable functional groups, such as basic and acidic groups. The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

When a residue (such as “R”) is defined herein as a single negative charge, then the residue can include a cationic counterion. The resulting salt form of the compound is encompassed in the structure as presented.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The graphic representations of racemic, ambiscalemic and scalemic or enantiomerically pure compounds used herein are taken from Maehr, J. Chem. Ed., 62: 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

The terms “enantiomeric excess” and diastereomeric excess” are used interchangeably herein. Compounds with a single stereocenter are referred to as being present in “enantiomeric excess,” those with at least two stereocenters are referred to as being present in “diastereomeric excess.” Preferred excesses are at least a percentage selected from 90%, 92%, 94%, 96% and 98%.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### Introduction

The present invention provides a class of luminescent probes that are based on metal ion (e.g., lanthanide, such as terbium and europium) chelates, which are formed between the metal ion and a novel class of macrocyclic ligands, such as those set forth in Formula I. These complexes exhibit high stability as well as high quantum yields of luminescence in aqueous media without the need for secondary activating agents such micelles or fluoride. Preferred ligands are macrocyclic structures incorporating an aromatic moiety, e.g., phthalamidyl, salicylamidyl, hydropyridinonate, etc., within their macrocyclic framework. The macrocycles of the invention are characterized by surprisingly high kinetic stability and unexpectedly low, non-specific binding to a variety of different polypeptides such as antibodies and proteins. These characteristics distinguish the macrocyclic structures of the invention from known, open-structured ligands.

Lanthanide complexes of the invention exhibit high quantum efficiencies and relatively high absorption coefficients. These properties make metal complexes of ligands of the invention useful for time resolved luminescence resonance energy transfer (TR-LRET) applications (e.g., homogeneous TR-LRET) in which donor and acceptor molecules are used at low concentrations. Complexes of the present invention find use in any application requiring strong luminescence under aqueous conditions including medical diagnostics and bioanalytical assay systems, such as immunoassays, peptide cleavage assays, DNA reporter assays and the like. In addition, these complexes and their derivatives have wide applicability in nanotechnology (incorporation into particles) and material science. In an exemplary embodiment, a complex of the invention is embedded in a solid material, allowing for the transmission of light.

Luminescent metal chelates of the invention can be used with other fluorophores or quenchers as components of energy transfer probes. Many fluorescent labels are useful in combination with the complexes of the invention and many such labels are available from commercial sources, such as SIGMA (Saint Louis) or Invitrogen, that are known to those of skill in the art. Furthermore, those of skill in the art will recognize how to select an appropriate fluorophore for a particular application and, if it is not readily available, will be able to synthesize the necessary fluorophore de novo or synthetically modify commercially available fluorescent compounds to arrive at the desired fluorescent label.

The compounds of the invention can be used as probes, as tools for separating particular ions from other solutes, as probes in microscopy, enzymology, clinical chemistry, molecular biology and medicine. The compounds of the invention are also useful as therapeutic agents and as diagnostic agents in imaging methods. Moreover, the compounds of the invention are useful as components of optical amplifiers of light, waveguides and the like. Furthermore, the compounds of the invention can be incorporated into inks and dyes, such as those used in the printing of currency and other instruments.

In one embodiment, the compounds of the invention show luminescence after exciting them in any manner known in the art, including, for example, with light or electrochemical energy (see, for example, Kulmala et al, Analytica Chimica Acta 1999, 386:1). The luminescence can, in the case of chiral compounds of the invention, be circularly polarized (see, for example, Riehl et al., Chem. Rev. 1986, 86:1). The present invention provides chiral chelates according to Formula I or II that are enantiomerically or diastereomerically enriched with respect to one enantionmer or diastereomer.

### Compositions

In a first embodiment, the invention provides a compound that includes a chelate structure according to Formula I:

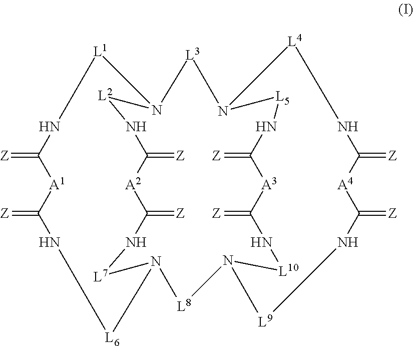

wherein the compound preferably includes at least one acceptor-linker, which is optionally covalently bound to a fluorophore, e.g., through a linkage fragment. In another embodiment, the compound of the invention includes at least one functional moiety. In a still further embodiment, a reactive functional group on the functional moiety is converted to a linkage fragment by reaction with a complementary reactive group on a carrier moiety, e.g., a nucleic acid, a peptide, an antibody, a saccharide, lectin, receptor, antigen, or a solid support.

In Formula I, each Z is a member independently selected from O and S. L¹, L², L³, L⁴, L⁵, L⁶, L⁷, L⁸, L⁹ and L¹⁰ (“L^{x}” moieties) are linker groups independently selected from substituted or unsubstituted alkyl, substituted or unsubstituted heteroalkyl, substituted or unsubstituted aryl, substituted or unsubstituted heteroaryl, substituted or unsubstituted cycloalkyl, and substituted or unsubstituted heterocycloalkyl.

A¹, A², A³ and A⁴ are members independently selected from substituted or unsubstituted aryl and substituted or unsubstituted heteroaryl (e.g., azulene) moieties. In an exemplary embodiment, these moieties are independently selected from the following structure:

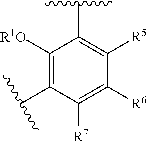

wherein each R¹ is a member independently selected from H, an enzymatically cleavable group, a hydrolytically cleavable group, a metabolically cleavable group and a single negative charge. Each R⁵, R⁶ and R⁷ (“an R^{x} moiety”) is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷R¹⁸, —C(O)R¹⁸—COOR¹⁷, —CONR¹⁷R¹⁸, —S(O)₂OR¹⁷, —OC(O)R¹⁷, —C(O)NR¹⁷R¹⁸, —NR¹⁷C(O)R¹⁸, —NR¹⁷SO₂R¹⁸, and —NO₂, wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl.

R¹⁷ and R¹⁸ are members independently selected from H, substituted or unsubstituted alkyl, substituted or unsubstituted heteroalkyl, substituted or unsubstituted aryl, substituted or unsubstituted heteroaryl, substituted or unsubstituted cycloalkyl, substituted or unsubstituted heterocycloalkyl, and R¹⁷ and R¹⁸, together with the atoms to which they are attached, are optionally joined to form a 5- to 7-membered ring.

In one embodiment, (a) a first moiety selected from L¹, L², L³, L⁴, L⁵, L⁶, L⁷, L⁸, L⁹, L¹⁰, R¹, R⁵, R⁶ and R⁷ comprises a acceptor-linker covalently attached to a fluorophore wherein said acceptor-linker and said fluorophore are covalently joined through a linkage fragment; or (b) a first moiety selected from L¹, L², L³, L⁴, L⁵, L⁶, L⁷, L⁸, L⁹, L¹⁰, R¹, R⁵, R⁶ and R⁷ comprises a acceptor-linker and a second moiety selected from L¹, L², L³, L⁴, L⁵, L⁶, L⁷, L⁸, L⁹, L¹⁰, R¹, R⁵, R⁶ and R⁷ comprises a functional moiety, wherein said first moiety and said second moiety are different moieties; or (c) a first moiety selected from L¹, L², L³, L⁴, L⁵, L⁶, L⁷, L⁸, L⁹, L¹⁰, R¹, R⁵, R⁶ and R⁷ comprises a acceptor-linker covalently joined, through a linkage fragment, to a fluorophore; and a second moiety selected from L¹, L², L³, L⁴, L⁵, L⁶, L⁷, L⁸, L⁹, L¹⁰, R¹, R⁵, R⁶ and R⁷ comprises a functional moiety wherein said first moiety and said second moiety, each selected from L¹, L², L³, L⁴, L⁵, L⁶, L⁷, L⁸, L⁹, L¹⁰, R¹, R⁵, R⁶ and R⁷, are different moieties; or (d) a first moiety selected from L¹, L², L³, L⁴, L⁵, L⁶, L⁷, L⁸, L⁹, L¹⁰, R¹, R⁵, R⁶ and R⁷ comprises a acceptor-linker; and a second moiety selected from L¹, L², L³, L⁴, L⁵, L⁶, L⁷, L⁸, L⁹, L¹⁰, R¹, R⁵, R⁶ and R⁷ comprises a functional moiety covalently joined, through a linkage fragment, to a member selected from a carrier moiety and a solid support wherein said first moiety and said second moiety, each selected from L¹, L², L³, L⁴, L⁵, L⁶, L⁷, L⁸, L⁹, L¹⁰, R¹, R⁵, R⁶ and R⁷, are different moieties; or (e) a first moiety selected from L¹, L², L³, L⁴, L⁵, L⁶, L⁷, L⁸, L⁹, L¹⁰, R¹, R⁵, R⁶ and R⁷ comprises a acceptor-linker covalently joined, through a linkage fragment, to a fluorophore; and a second moiety selected from L¹, L², L³, L⁴, L⁵, L⁶, L⁷, L⁸, L⁹, L¹⁰, R¹, R⁵, R⁶ and R⁷ comprises a functional moiety covalently joined, through a linkage fragment, to a member selected from a carrier moiety and a solid support wherein said first moiety and said second moiety, each selected from L¹, L², L³, L⁴, L⁵, L⁶, L⁷, L⁸, L⁹, L¹⁰, R¹, R⁵, R⁶ and R⁷ are different moieties a first moiety selected from L¹, L², L³, L⁴, L⁵, L⁶, L⁷, L⁸, L⁹, L¹⁰, R¹, R⁵, R⁶ and R⁷ comprises a moiety which is both a fluorescent-linker and a functional moiety; or (f) a first moiety selected from L¹, L², L³, L⁴, L⁵, L⁶, L⁷, L⁸, L⁹, L¹⁰, R¹, R⁵, R⁶ and R⁷ comprises a moiety which is both a fluorescent-linker and a functional moiety covalently bound, through at least one linkage fragment to at least one member selected from a fluorophore, a carrier moiety, a solid support and a combination thereof, wherein each of said at least one linkage fragment is the same or different.

In an exemplary embodiment, a compound according to Formula I is covalently attached through the functional moiety to a carrier molecule.

Exemplary compounds according to any of the embodiments discussed above, are those in which at least one L^{x} or R^{x} moiety is functionalized with a acceptor-linker, optionally bound to a fluorophore, e.g., through a linkage fragment. In another embodiment, at least one of L^{x} or R^{x} moiety is functionalized with a functional moiety (optionally bound to a carrier moiety or solid support, e.g., through a linkage fragment). In yet another exemplary embodiment, one of these L^{x} or R^{x} groups is functionalized with a acceptor-linker (optionally bound to a fluorophore) and the same or a different L^{x} or R^{x} moiety is functionalized with a functional moiety (optionally bound to a carrier moiety or solid support).

Thus, the present invention provides compounds according to Formula I in which at least one L^{x} moiety is substituted with a group selected from:

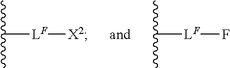

in which L^{F} is a acceptor-linker as described herein, and X² is a reactive functional group. F is a fluorophore bound to L^{F} through a linkage fragment formed as described herein.

In another embodiment, the invention provides compounds according to Formula I in which at least one L^{x} moiety is substituted with a group selected from:

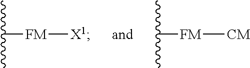

in which FM is a functional moiety as described herein, having as a component a reactive functional group, X¹. CM is a carrier moiety (or solid support) bound to FM through a linkage fragment formed as described herein.

In still a further embodiment, the invention provides a compound according to Formula I in which at least one L^{x} moiety is substituted with a group selected from:

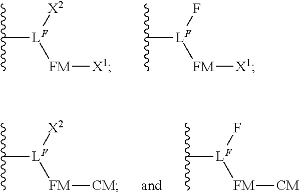

in which the moieties are as described above. As will be appreciated by those of skill in the art, rather than a fluorophore, the acceptor-linker can be conjugated to one or more quencher or other luminescence modifying moiety.

In an exemplary embodiment, the present invention provides a composition comprising a chelate according to Formula I, combined with a fluorophore. The chelate and the fluorophore are preferably both linked to a carrier moiety: each can be linked to the same carrier moiety or to a different carrier moiety. It is generally preferred that the chelate be complexed with a metal ion selected such that the chelate forms an energy transfer pair with the fluorophore. In general, the metal complex will serve as the donor fluorophore, and will have a longer excited state lifetime than the acceptor fluorophore. In an exemplary embodiment, the donor fluorophore is a lanthanide chelate. In another exemplary embodiment, the acceptor fluorophore is an organic fluorophore, e.g., a polyaromatic hydrocarbon (e.g., a heterocyclic compound).

Transfer of excited state energy from the donor fluorophore to the acceptor fluorophore, provides an acceptor fluorophore with a longer excited state lifetime than and identical fluorophore that is not excited by the donor. The acceptor fluorophore generally luminesces at a wavelength longer than that of the energy incoming from the donor.

In another exemplary embodiment, the compositions of the invention include multiple donor fluorophores. In a further embodiment, the compositions of the invention include multiple acceptor fluorophores. The compositions can include both multiple donor and multiple acceptor fluorophores (or quenchers or other luminescence modifying moieties).

The luminescent complexes according to Formula I, in conjunction (e.g., operative contact allowing exchange of energy) with energy transfer to a fluorophore, provides a luminescent system that is tunable with respect to emission wavelength. The emission wavelength is tunable because, when energy transfer is chosen to be large, emission color is principally determined by the emission wavelength of the fluorophore, which can be selected for its output color.

The complexes in conjunction with the fluorophore are also tunable with respect to emission lifetime because the lifetime is determined by the efficiency of energy transfer from the complex of Formula Ito the fluorophore. The fluorophore typically has a short lifetime. Because it is continuously excited by the luminescent complex of Formula I, its emission intensity decays with a lifetime related to the lifetime of the luminescent complex. The lifetime can be tuned by altering the distance between the luminescent complex and the fluorophore. The Foerster equation is of use to predict the lifetime of the energy transfer pair.

In another exemplary embodiment, the compound of the invention has the structure:

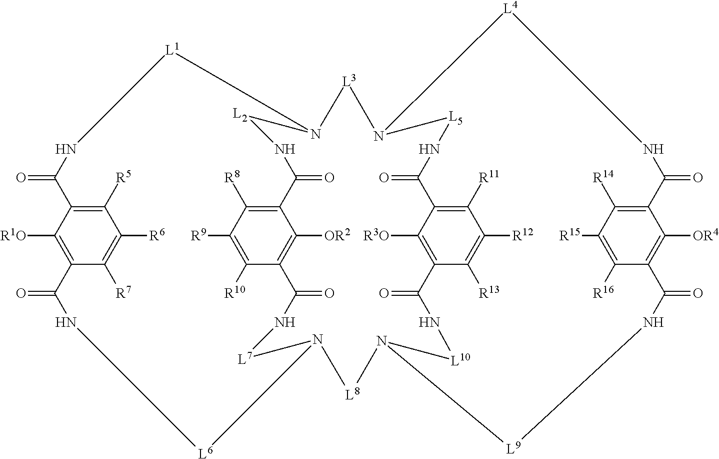

wherein R¹, R², R³ and R⁴ are members independently selected from H, an enzymatically cleavable group, a hydrolytically cleavable group, a metabolically cleavable group, a group that is cleaved by incident light and a single negative charge. The substituents and attributes of compounds according to this embodiment are as described above with reference to Formula I. Any one or more than one of L¹, L², L³, L⁴, L⁵, L⁶, L⁷, L⁸, L⁹ and L¹⁰ can be substituted with one or more functional moiety and/or acceptor-linker.

In another exemplary embodiment, the linker moieties L¹, L², L³, L⁴, L⁵, L⁶, L⁷, L⁸, L⁹ and L¹⁰ are members independently selected from substituted or unsubstituted C₁ to C₆ alkyl. Exemplary compounds include those in which L¹, L², L³, L⁴, L⁵, L⁶, L⁷, L⁸, L⁹ and L¹⁰ are members independently selected from substituted or unsubstituted ethyl. An exemplary ligand according to this embodiment has the structure:

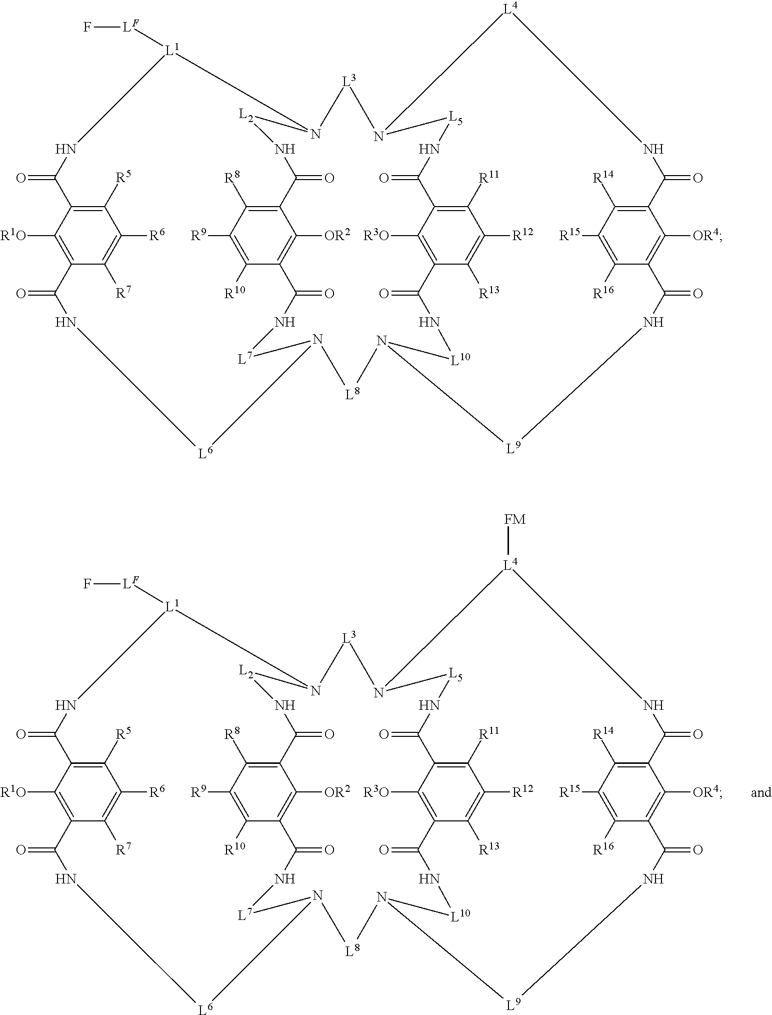

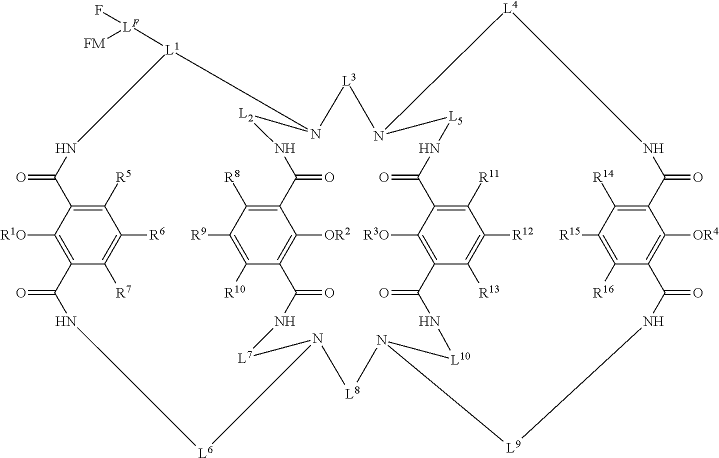

wherein L^{F} is the acceptor-linker, F represents the fluorophore and FM is the functional moiety. As will be appreciated by those of skill, the acceptor-linker and functional moiety can be attached to any one or more than one of L¹, L², L³, L⁴, L⁵, L⁶, L⁷, L⁸, L⁹ and L¹⁰.

In another embodiment, the compound of the invention has a formula selected from:

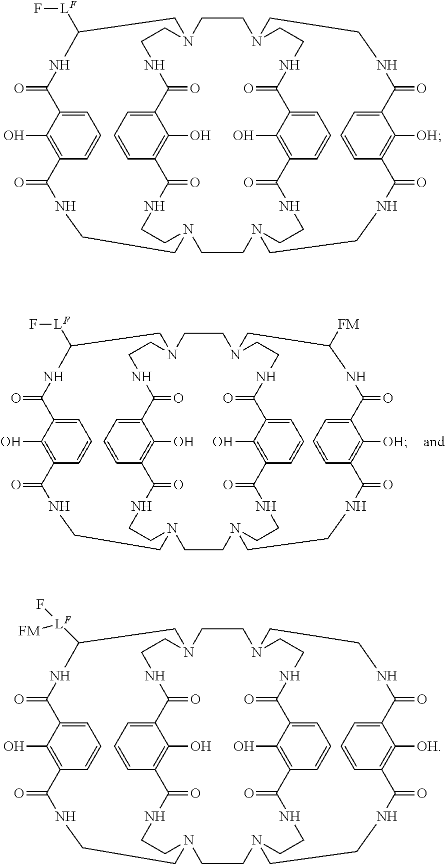

Each of the structures above is intended to include those derivatives in which the acceptor-linker is not conjugated to a fluorophore as well as those conjugated to a fluorophore. Also included are those derivatives in which the functional moiety is conjugated to a carrier moiety (CM) as well as those that are conjugated to a carrier moiety or solid support.

In another exemplary embodiment, the invention provides a compounds having the formula:

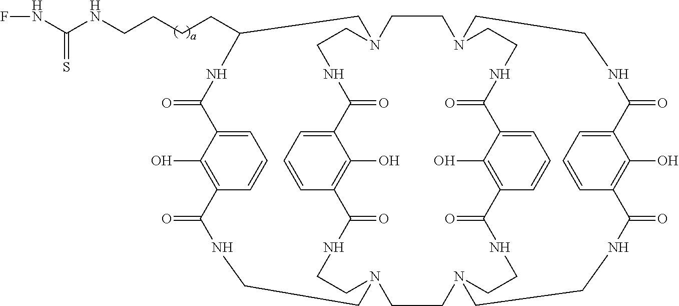

in which a is an integer greater than or equal to 0, e.g., from 0 to 10.

An exemplary compound according to this embodiment has the formula:

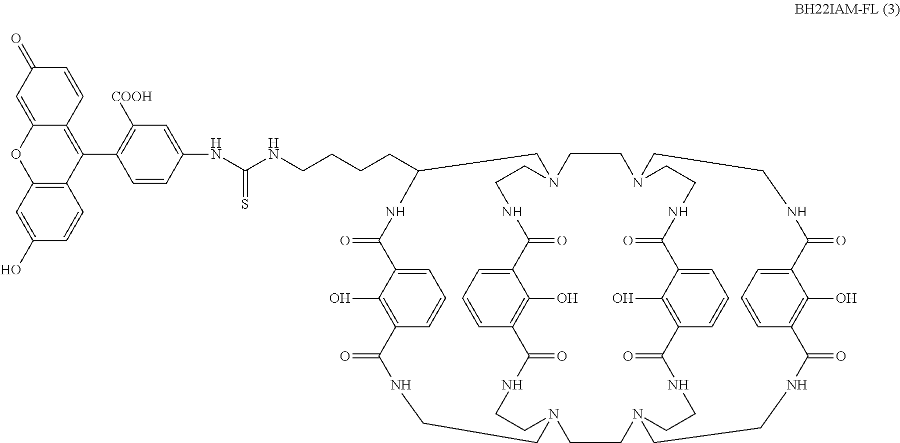

In another exemplary embodiment, the compound of the invention includes and amide linkage, which is more stable than the thiourea:

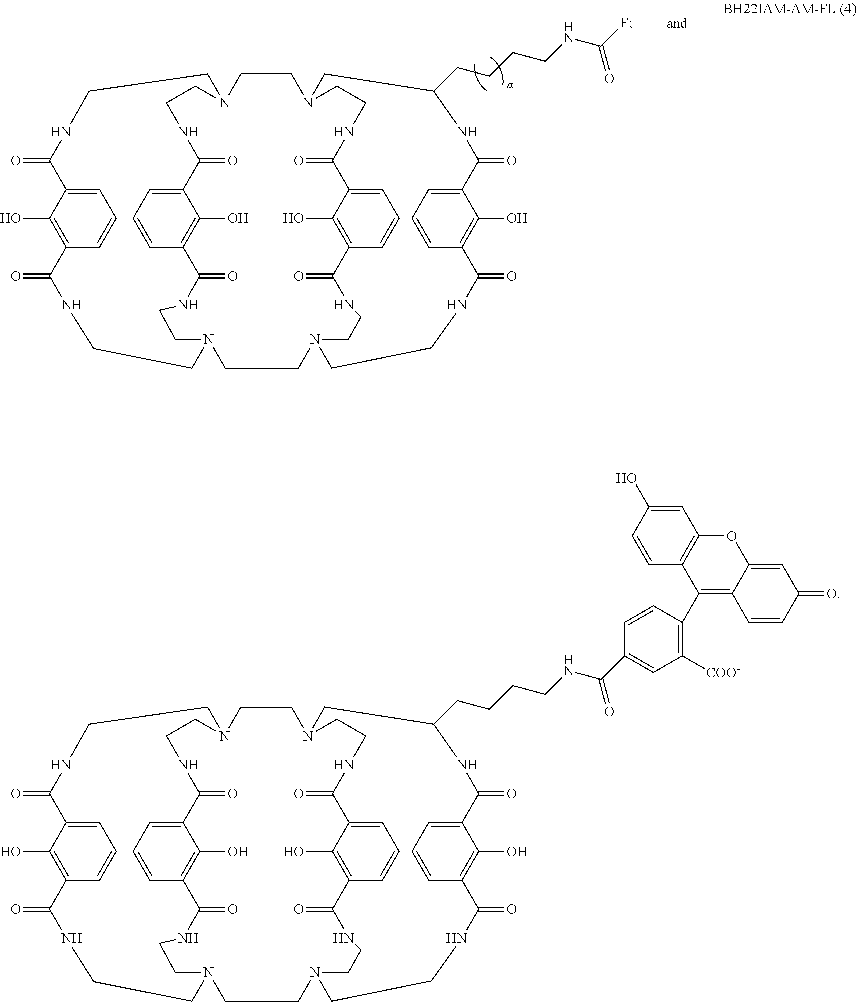

Compounds of the inventions including both a acceptor-linker moiety, optionally attached to a fluorophore, and a functional moiety, optionally attached to a carrier moiety (or solid support) are exemplified by the following compounds:

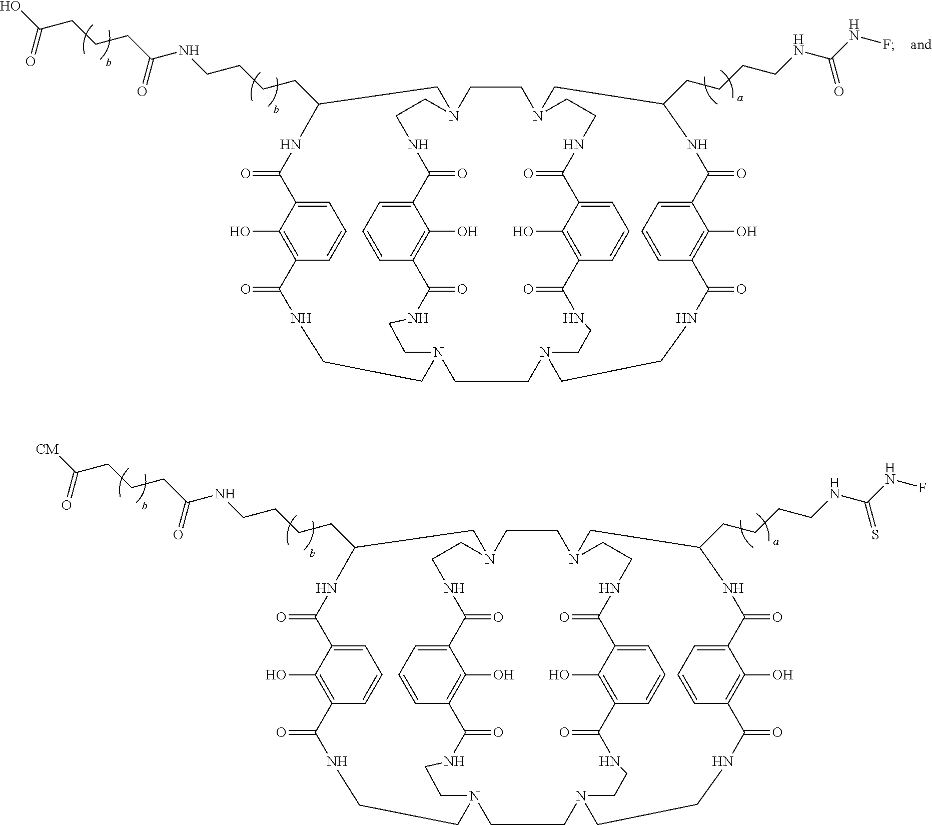

in which a and b are independently selected from 0, 1, 2, 3, 4, 5, 6, 7, 8, 9, 10, 11, 12, 13, 14 and 15; and CM and F are as described above.

Other exemplary compound of the invention in which F is an organic fluorophore and the acceptor-linker and FM are attached at different sites on the compound according to Formula I have the formulae:

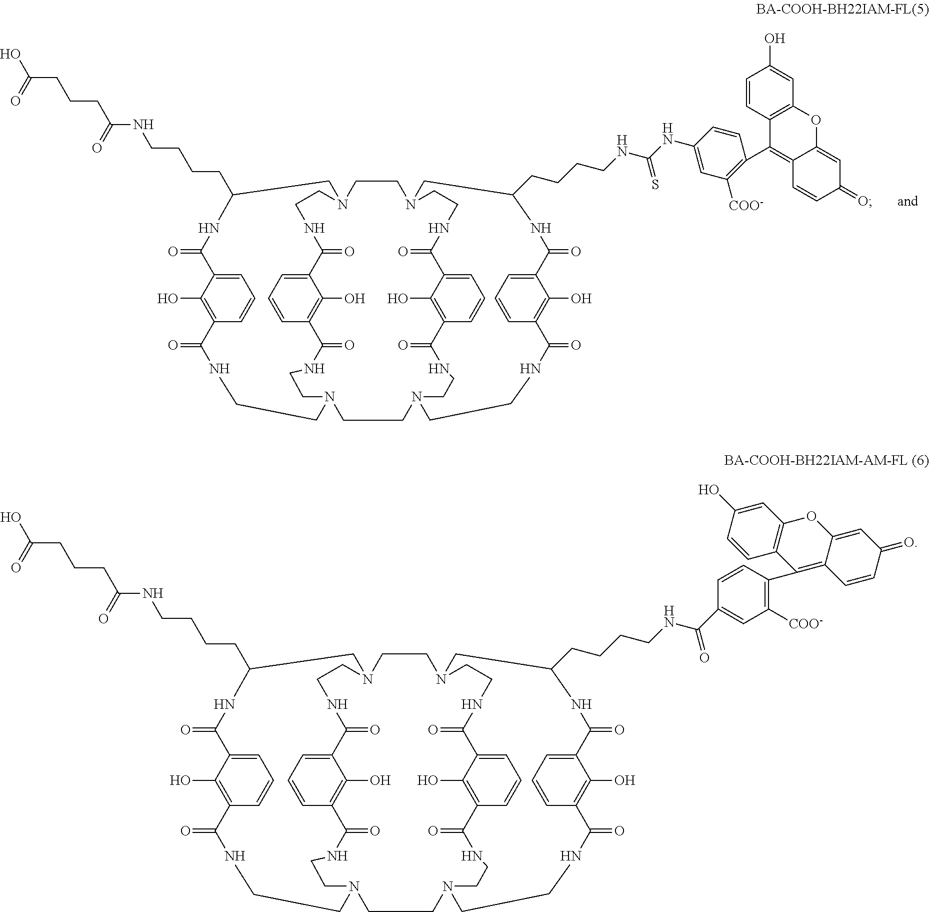

In still other exemplary embodiments, the functional moiety and the acceptor-linker are components of a structure bonded at the same point (e.g., the same atom) of the chelate, providing exemplary compounds having the formulae:

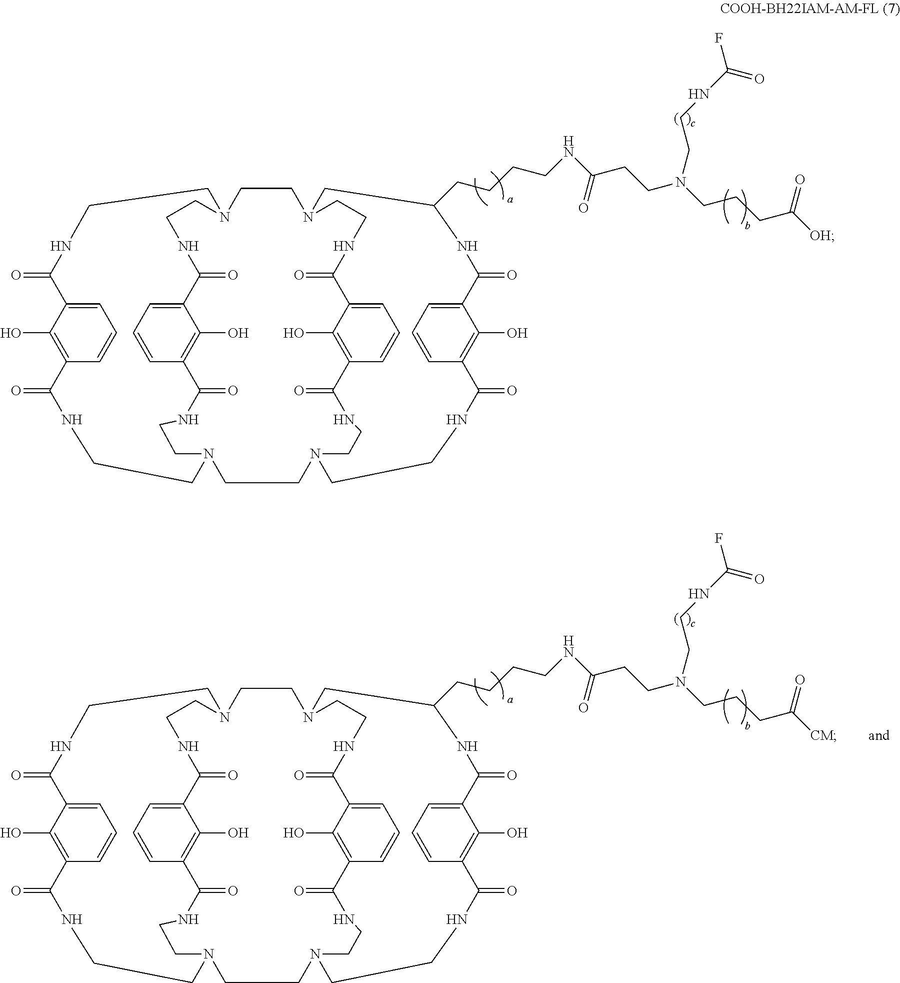

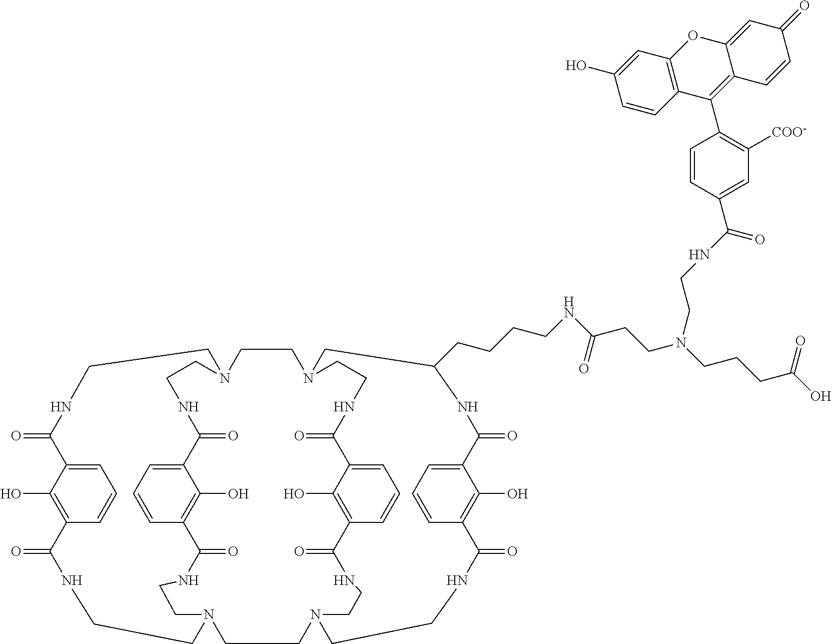

in which a, b and c are integers independently selected from 0, 1, 2, 3, 4, 5, 6, 7, 8, 9, 10, 11, 12, 13, 14 and 15; CM is a carrier moiety (or solid support) and F is a fluorophore. Any of the compounds disclosed above and elsewhere herein (in particular, a macrocyclic compound) can be functionalized or otherwise modified for attachment to additional moieties to form a luminescent compound or luminescent group, such as an energy transfer donor or an energy transfer acceptor.

Also provided are metal complexes formed from each of the chelates of the invention described herein. In an exemplary embodiment, the metal ion is selected to provide a metal chelate that is capable of transferring energy to the fluorophore. Exemplary metal ions of use in to transfer energy to a fluorophore in compounds of this invention are lanthanide ions.

In another exemplary embodiment, the compounds of the invention emit light at an emission wavelength of the fluorophore attached to the metal chelate through the acceptor-linker. Exemplary compounds of the invention are characterized by emitting at a wavelength characteristic of the fluorophore, and the emission having a significantly enhanced lifetime. For example, compound 3, when coordinated to terbium and excited at a BH22IAM absorption wavelength (˜340 nm), the metal chelate emits at 520 nm, the characteristic wavelength of fluorescein with a lifetime of 524 ns. The new lifetime is over 100-fold longer than the lifetime for a fluorescein solution that is directly excited (<5 ns).

Probes based on the nucleotide sequences can be used to detect or amplify transcripts or genomic sequences encoding the same or homologous proteins. In other embodiments, the probe further comprises a label group attached thereto, e.g., the label group can be a radioisotope, an enzyme, or an enzyme co-factor. Such probes can be used as a part of a genomic marker test kit for identifying cells which express a particular protein, such as by measuring a level of the protein-encoding nucleic acid in a sample of cells, e.g., detecting the target nucleic acid mRNA levels or determining whether the gene encoding the mRNA has been mutated or deleted.

In generality, a nucleic acid probe of the invention (sometimes referred to as a “capture probe”) comprises a nucleic acid probe sequence that hybridizes, e.g., hybridizes under stringent conditions, to a target nucleotide sequence of interest. These hybridization conditions include washing with a solution having a salt concentration of about 0.02 molar at pH 7 at about 60° C. As used herein, the term “hybridizes under stringent conditions” is intended to describe conditions for hybridization and washing under which nucleotide sequences at least 60% complementary to each other typically remain hybridized to each other. Preferably, the conditions are such that sequences having perfect complementary or a complementarity of at least a percentage selected from 70%, 80%, 90%, 95%, 96%, 97%, 98%, 99% typically remain hybridized to each other. Such stringent conditions are known to those skilled in the art and can be found in Current Protocols in Molecular Biology, John Wiley & Sons, N.Y. (1989) 6.3.1 6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. As used herein, a “naturally-occurring” nucleic acid molecule refers to a RNA or DNA molecule having a nucleotide sequence that occurs in nature (e.g., encodes a natural protein).

The nucleic acid probes of the invention can be DNA or RNA or chimeric mixtures or derivatives or modified versions thereof, so long as it is still capable of hybridizing to the desired target nucleic acid. In addition to being labeled with a resonance energy transfer moiety, the nucleic acid sequence can be modified at the base moiety, sugar moiety, or phosphate backbone, and may include other appending groups or labels, so long as it is still capable of priming the desired amplification reaction, or functioning as a blocking oligonucleotide, as the case may be.

For example, a nucleic acid probe of the present invention can be chemically synthesized using naturally occurring nucleotides or variously modified nucleotides designed to increase the biological stability of the molecules or to increase the physical stability of the duplex formed between the complimentary nucleic acids, e.g., phosphorothioate derivatives and acridine substituted nucleotides can be used. A preferred example of a class of modified nucleotides which can be used to generate the nucleic acid probes is a 2′-O-methyl nucleotide. Additional examples of modified nucleotides which can be used to generate the nucleic acid probes include for example 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In another embodiment, the nucleic acid probe of the present invention comprises at least one modified sugar moiety selected from the group including but not limited to arabinose, 2-fluoroarabinose, xylulose, and hexose. In yet another embodiment, the nucleic acid probe of the present invention comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. As stated above, a preferred example of a modified nucleotide which can be used to generate the nucleic acid probes is a 2′-O-methyl nucleotide.

Nucleic acid probes of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (Nucl. Acids Res., 1988, 16: 3209), methylphosphonate oligonucleotides can be prepared by use of controlled pore glass polymer supports (Sarin et al., Proc. Natl. Acad. Sci. U.S.A., 1988, 85: 7448-7451), etc.

Once the desired oligonucleotide is synthesized, it is cleaved from the solid support on which it was synthesized and treated, by methods known in the art, to remove any protecting groups present. The oligonucleotide may then be purified by any method known in the art, including extraction and gel purification. The concentration and purity of the oligonucleotide may be determined by examining oligonucleotide that has been separated on an acrylamide gel, or by measuring the optical density at 260 nm in a spectrophotometer.

Nucleic acid probes of the invention may be labeled with donor and acceptor moieties during chemical synthesis or the label may be attached after synthesis by methods known in the art. In a specific embodiment, the following donor and acceptor pairs are used: a luminescent lanthanide chelate, e.g., terbium chelate or lanthanide chelate, is used as the donor, and an organic dye such as fluorescein, rhodamine or CY-5, is used as the acceptor. Preferably, terbium is used as a donor and fluorescein or rhodamine as an acceptor, or europium is used as a donor and CY-5 as an acceptor. In another specific embodiment, the donor is fluorescent, e.g. fluorescein, rhodamine or CY-5, and the acceptor is luminescent, e.g. a lanthanide chelate. In yet another embodiment, the energy donor is luminescent, e.g., a lanthanide chelate, and the energy acceptor may be non-fluorescent.

One of ordinary skill in the art can easily determine, using art-known techniques of spectrophotometry, which fluorophores will make suitable donor-acceptor FRET pairs. For example, FAM (which has an emission maximum of 525 nm) is a suitable donor for TAMRA, ROX, and R6G (all of which have an excitation maximum of 514 nm) in a FRET pair. Probes are preferably modified during synthesis, such that a modified T-base is introduced into a designated position by the use of Amino-Modifier C6 dT (Glen Research), and a primary amino group is incorporated on the modified T-base, as described by Ju et al. (1995, Proc. Natl. Acad. Sci. USA 92:4347 4351). These modifications may be used for subsequent incorporation of fluorescent dyes into designated positions of the nucleic acid probes of the present invention.

The optimal distance between the donor and acceptor moieties will be that distance wherein the emissions of the donor moiety are maximally absorbed by the acceptor moiety. This optimal distance varies with the specific moieties used, and may be easily determined by one of ordinary skill in the art using well-known techniques. The lifetime of the luminescence of the compounds of the invention is readily tuned by varying the distance between the luminescent complex and the fluorophore. For energy transfer in which the fluorophore that emits energy is to be detected, the donor and acceptor fluorophores are preferably separated when hybridized to target nucleic acid by a distance of up to 30 nucleotides, more preferably from about 1 to about 20 nucleotides, and still more preferably from about 2 to about 10 nucleotides and more preferably separated by 3, 4, 5, 6, 7, 8 or 9 nucleotides. For energy transfer wherein it is desired that the acceptor moiety quench the emissions of the donor, the donor and acceptor moieties are preferably separated by a distance of less than 10, 9, 8, 7, 6, 5, 4, 3, 2, or 1 nucleotide(s) (e.g., on the opposite strand, complementary nucleotides of a duplex structure), although a 5 nucleotide distance (one helical turn) is also advantageous for use.

The nucleic acid probes of the invention have use in nucleic acid detection, or amplification reactions as primers, or in the case of triamplification, blocking oligonucleotides, to detect or measure a nucleic acid product of the amplification, thereby detecting or measuring a target nucleic acid in a sample that is complementary to a 3′ primer sequence. Accordingly, the nucleic acid probes of the invention can be used in methods of diagnosis, wherein a sequence is complementary to a sequence (e.g., genomic) of an infectious disease agent, e.g. of human disease including but not limited to viruses, bacteria, parasites, and fungi, thereby diagnosing the presence of the infectious agent in a sample of nucleic acid from a patient. The target nucleic acid can be genomic or cDNA or mRNA or synthetic, human or animal, or of a microorganism, etc.

### Functional Moiety/Acceptor-Linker

In some embodiments, a compound of the invention comprises at least one functional moiety. In some embodiments, a reactive functional group on the functional moiety is converted to a linkage fragment by reaction with a complementary reactive group on a carrier moiety.

The compounds of the invention include one or more structure referred to herein as a functional moiety and acceptor-linker. These moieties have a structure appropriate to allow their covalent attachment to a carrier moiety (or solid support) or a fluorophore (e.g., an organic fluorophore), respectively. Prior to conjugation with a fluorophore or carrier moiety (or solid support), the acceptor-linker and the functional moiety include a reactive functional group.

In a further embodiment, the acceptor-linker and/or the functional moiety is bound to a fluorophore or carrier moiety (or solid support), respectively. Binding of the fluorophore or carrier moiety is effected through reaction of complementary functional groups on the fluorophore, or carrier moiety, and the acceptor-linker or functional moiety, respectively, thereby forming a linkage fragment which joins the two components. Exemplary linkage fragments include: S, SC(O)NH, SC(O)(NH)₂, HNC(O)S, SC(O)O, O, NH, NHC(O), (NH)₂C(O), (O)CNH and NHC(O)O, and OC(O)NH, CH₂S, CH₂O, CH₂CH₂O, CH₂CH₂S, (CH₂)ₚO, (CH₂)ₚS or (CH₂)ₚY′-PEG wherein, Y′ is S, NH, NHC(O), C(O)NH, NHC(O)O, OC(O)NH, or O and p is an integer from 1 to 50.

The acceptor-linker and functional moiety can be of any useful structure including, but not limited to, acyl, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, peptide (e.g., a peptide including a protease site), nucleic acid (e.g., hybridization probes, PCR primers), saccharide (e.g., dextran, starch, cyclodextrin). In one preferred embodiment, the linker L¹¹ of the functional moiety is long enough to avoid side reactions during synthesis (e.g. intra-molecular reactions, such as intra-molecular peptide bond formation), to allow coupling of the compound or complex of the invention to a targeting moiety and to allow the targeting moiety to fulfill its intended function. Useful linkers include those with about 2 to about 50 linear atoms, preferably about 4 to about 20 linear atoms.

In an exemplary embodiment, the acceptor-linker is a nucleic acid and the invention provides a probe based on the nucleic acid. In an example according to this embodiment, an oligonucleotide probe is labeled with a luminescent chelate of the invention as the donor, and an organic fluorophore as the acceptor (reporter) moiety. The nucleic acid probe in a LRET pair can be a simple linear probe, i.e., neither a quencher nor a hairpin structure is necessary.

In one exemplary embodiment, the compounds of the invention are derivatized with a functional moiety. The functional moiety can, for example, be attached to one of the linker units or to one of the building blocks. When two or more functional moieties are used, each can be attached to any of the available linking sites.

The acceptor-linker and/or the functional moiety is preferably attached, so that the resulting functionalized ligand will be able to undergo formation of stable metal ion complexes. In an exemplary embodiment, the macrocyclic ligand is derivatized with a functional moiety. Formula II below shows preferred sites for derivatization with a functional group and/or a acceptor-linker of the chelates of the invention.

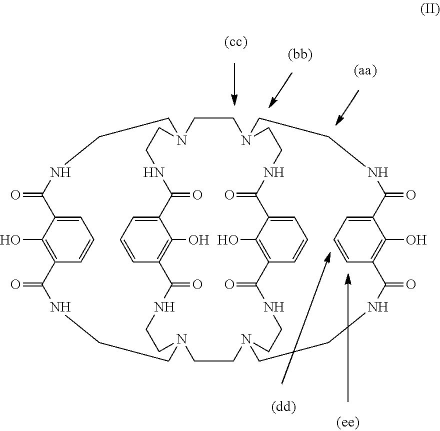

In one exemplary embodiment, a compound according to Formula II is derivatized at position (aa), (bb) or (cc). However, ligands, in which alternative positions within the core structure of the ligand (e.g., positions (dd) and (ee)) are derivatized with a functional moiety and/or a acceptor-linker have similarly useful properties and are encompassed within the instant invention. Those of skill will appreciate that the substitution strategy set forth above is broadly relevant to all compounds according to Formula I, including those compounds set forth herein.

In an exemplary embodiment, the functional moiety (or acceptor-linker) has the structure:

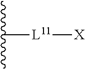

wherein L¹¹ is a linker moiety, which is a member selected from a bond, acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. X is a reactive functional group, which can be reacted with a carrier moiety (or solid support) or a fluorophore, conjugating this species to the linker through a linkage fragment. In some embodiments, X is a linkage fragment.

Exemplary ligands that include a functional moiety have the structure:

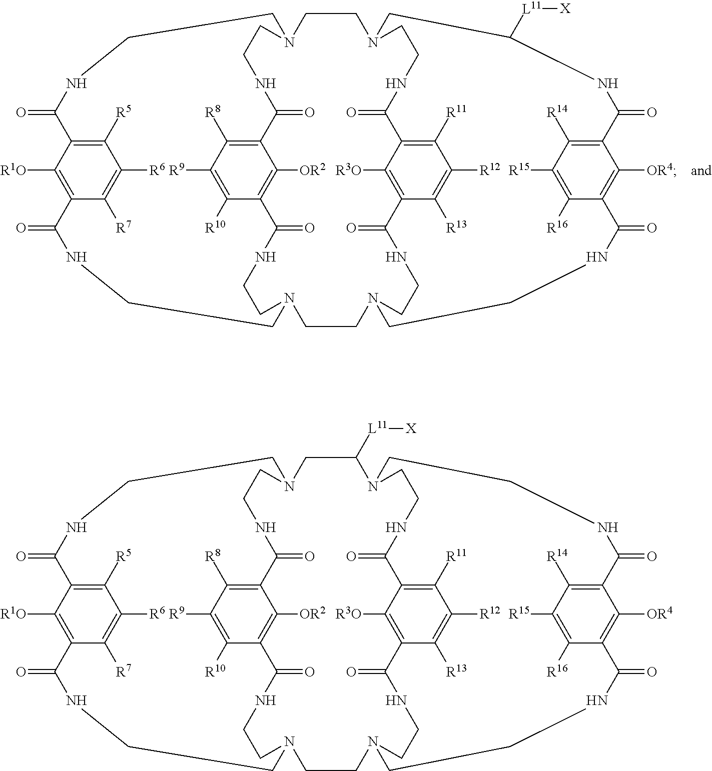

wherein L¹¹, X, R⁵, R⁶, R⁷, R⁸, R⁹, R¹⁰, R¹¹, R¹², R¹³, R¹⁴, R¹⁵ and R¹⁶ are as defined above.

In one example, R⁵ to R¹⁶ are H. Exemplary ligands have the structure:

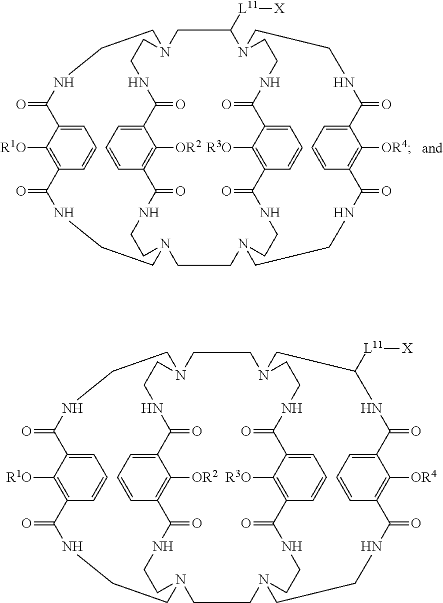

Functionalization of a compound according to Formula II at position (aa) with a (CH₂)₄NH₂ group leads to the macrocyclic derivative:

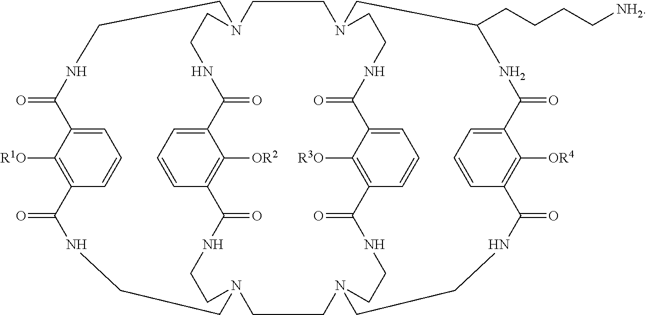

### Reactive Functional Groups

In one embodiment, the functional moiety includes a reactive functional group, which can be used to covalently attach the ligand to another species, e.g. a carrier moiety or solid support. Alternatively, the reactive functional group can be used to link the ligand to a nano-particle of any kind Reactive groups and classes of reactions useful in practicing the present invention are generally those that are well known in the art of bioconjugate chemistry. Currently favored classes of reactions available with reactive functional groups of the invention are those which proceed under relatively mild conditions. These include, but are not limited to nucleophilic substitutions (e.g., reactions of amines and alcohols with acyl halides and activated esters), electrophilic substitutions (e.g., enamine reactions) and additions to carbon-carbon and carbon-heteroatom multiple bonds (e.g., Michael reactions and Diels-Alder reactions). These and other useful reactions are discussed, for example, in: March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

### a) Amines and Amino-Reactive Groups

In one embodiment, the reactive functional group is a member selected from amines, such as a primary or secondary amine, hydrazines, hydrazides, and sulfonylhydrazides. Amines can, for example, be acylated, alkylated or oxidized. Useful non-limiting examples of amino-reactive groups include N-hydroxysuccinimide (NHS) esters, sulfur-NHS esters, imidoesters, isocyanates, isothiocyanates, acylhalides, arylazides, p-nitrophenyl esters, aldehydes, sulfonyl chlorides and carboxyl groups.

NHS esters and sulfur-NHS esters react preferentially with the primary (including aromatic) amino groups of the reaction partner. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide.

Imidoesters are the most specific acylating reagents for reaction with the amine groups of e.g., a protein. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the reaction partner attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of carboxylic acids are also useful amino-reactive groups. Although the reagent specificity is not very high, α- and ε-amino groups appear to react most rapidly.

Aldehydes react with primary amines of the conjugate components (e.g., ε-amino group of lysine residues). Although unstable, Schiff bases are formed upon reaction of the protein amino groups with the aldehyde. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product. Alternatively, a stable bond may be formed by reductive amination.

Aromatic sulfonyl chlorides react with a variety of sites of the conjugate components, but reaction with the amino groups is the most important, resulting in a stable sulfonamide linkage.

Free carboxyl groups react with carbodiimides, soluble in both water and organic solvents, forming pseudoureas that can then couple to available amines yielding an amide linkage. Yamada et al., Biochemistry 1981, 20: 4836-4842, e.g., teach how to modify a protein with carbodiimides.

### b) Sulfhydryl and Sulfhydryl-Reactive Groups

In another embodiment, the reactive functional group is a member selected from a sulfhydryl group (which can be converted to disulfides) and sulfhydryl-reactive groups. Useful non-limiting examples of sulfhydryl-reactive groups include maleimides, alkyl halides, acyl halides (including bromoacetamide or chloroacetamide), pyridyl disulfides, and thiophthalimides.

Maleimides react preferentially with the sulfhydryl group of the conjugate components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryl groups via disulfide exchange to give mixed disulfides. As a result, pyridyl disulfides are relatively specific sulfhydryl-reactive groups.

Thiophthalimides react with free sulfhydryl groups to also form disulfides.

### c) Other Reactive Functional Groups

Other exemplary reactive functional groups include:
(a) carboxyl groups and various derivatives thereof including, but not limited to, N-hydroxybenztriazole esters, acid halides, acyl imidazoles, thioesters, p-nitrophenyl esters, alkyl, alkenyl, alkynyl, including succinic and maleic active esters and aromatic esters;
(b) hydroxyl groups, which can be converted to esters, ethers, aldehydes, etc.;
(c) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) alkenes, which can undergo, for example, cycloadditions, acylation, Michael addition, etc;
(g) epoxides, which can react with, for example, amines and hydroxyl groups;
(h) phosphoramidites and other standard functional groups useful in nucleic acid synthesis and
(i) any other functional group useful to form a covalent bond between the functionalized ligand and a molecular entity or a surface.
   d) Functional Groups with Non-Specific Reactivities

In addition to the use of site-specific reactive moieties, the present invention contemplates the use of non-specific reactive groups to link the ligand to a targeting moiety. Non-specific groups include photoactivatable groups, for example.

Photoactivatable groups are ideally inert in the dark and are converted to reactive species in the presence of light. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming intraprotein crosslinks.

It is well within the abilities of a person skilled in the art to select a reactive functional group, according to the reaction partner. As an example, an activated ester, such as an NHS ester will be useful to label a protein via lysine residues. Sulfhydryl reactive groups, such as maleimides can be used to label proteins via amino acid residues carrying an SH-group (e.g., cystein). Antibodies may be labeled by first oxidizing their carbohydrate moieties (e.g., with periodate) and reacting resulting aldehyde groups with a hydrazine containing ligand.

The reactive functional groups can be chosen such that they do not participate in, or interfere with, the reactions necessary to assemble the reactive ligand. Alternatively, a reactive functional group can be protected from participating in the reaction by means of a protecting group. Those of skill in the art understand how to protect a particular functional group so that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

With respect to each of the functional groups set forth above, when the functional group is on a linker, it is generally preferred that the functional group is located at a terminus of the linker. Thus, it is generally preferred that the functional group on the functional moiety and the acceptor-linker are found at a terminus of the functional moiety and the acceptor-linker, respectively.

Exemplary compounds of the invention include a compound according to

Formula I, wherein the compound comprises -L¹¹-X having a structure selected from

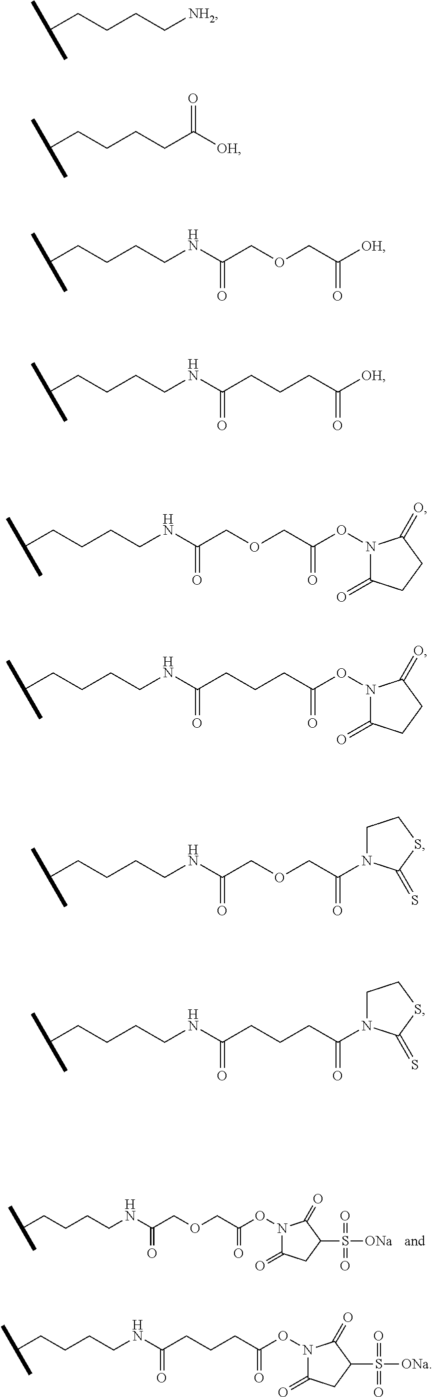

In some embodiments, -L¹¹-X is selected from

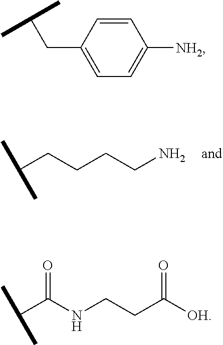

In the drawings of these moieties, the thickened leftmost bar represents the rest of the molecule to which the moiety is attached. As will be apparent to those of skill in the art, the amines and carboxylic acids of the precursor compounds are readily covalently bound through a linkage fragment to one or more carrier moiety, solid support, or fluorophore. In some embodiments, X is a linkage fragment, which itself is bonded to a protein, such as a DNA polymerase. The moieties -L¹¹-X shown above or any -L¹¹-X described herein can be attached to a macrocycle described herein to act as a linker between the macrocycle and any other useful entity.

### Targeting Moieties

Exemplary targeting moieties include carrier molecules as discussed herein; including small-molecule ligands, lipids, linear and cyclic peptides, polypeptides (e.g., EPO, insulin etc.), enzymes, antibodies and receptors. Other targeting moieties include antibody fragments (e.g., those generated to recognize small-molecules and receptor ligands), antigens, nucleic acids (e.g. RNA and cDNA), carbohydrate moieties (e.g., polysaccharides), and pharmacologically active molecules, such as toxins, pharmaceutical drugs and drugs of abuse (e.g. steroids). Additional targeting moieties are selected from solid supports and polymeric surfaces (e.g., polymeric beads and plastic sample reservoirs, such as plastic well-plates), sheets, fibers and membranes. Targeting moieties also include particles (e.g., nano-particles) and drug-delivery vehicles.

In one embodiment, the targeting moiety includes at least one unit of a macrocyclic compound. In an exemplary embodiment, the macrocyclic compound of the targeting moiety has a structure according to Formula I. In another exemplary embodiment, the compound of the invention has a dendrimeric structure and encompasses several ligands having a structure according to Formula I. In a further exemplary embodiment, according to this aspect, a complex based on such dendrimer includes at least two metal ions.

In one exemplary embodiment, the targeting moiety is substituted with a luminescence modifying group that allows luminescence energy transfer between a complex of the invention and the luminescence modifying group when the complex is excited.

In another exemplary embodiment, the linker moiety L¹¹ or the targeting moiety includes an ether or polyether, such as polyethylene glycol (PEG) and derivatives thereof. In one example, the polyether has a molecular weight between about 50 to about 10,000 daltons.

In further embodiments, the compounds and luminescent complexes of the invention can be used in any assay format aimed at detecting a lipid in a sample (e.g., in the blood of a patient). An exemplary complex according to this embodiment, includes a targeting moiety, which is a protein containing a lipid recognition motif. Exemplary lipid binding proteins include those that bind to phosphatidylinositol, phosphatidylinositol phosphates or other biological lipids.

In another example, the targeting moiety is an antibody that recognizes and binds to an analyte. In an exemplary assay system an analyte may be detected in a sample by first incubating the sample with a complex of the invention, wherein the complex is covalently bound to an antibody that includes a binding site for the analyte. To the mixture can then be added an excess of a probe molecule that binds to the same binding site as the analyte and includes a luminescence modifying group (e.g. an acceptor). The presence and concentration of analyte in the sample is indicated by the luminescence of the assay mixture. For instance, if the concentration of analyte in the sample is high, many of the antibody binding sites will be occupied with the analyte and less binding sites will be available for the probe molecule. In an exemplary embodiment, the analyte is a lipid molecule.

### Complexes

The invention provides complexes formed between at least one metal ion and a compound according to Formula I. Exemplary complexes are luminescent, and the metal ion is chosen according to meeting this criterion. In one exemplary embodiment, the metal is a member selected from the lanthanide group and the complex is preferably luminescent. Exemplary lanthanides include neodynium (Nd), samarium (Sm), europium (Eu), gadolinium (Gd), terbium (Tb), dysprosium (Dy) and ytterbium (Yb), of which europium and terbium are presently preferred.

### Fluorophore (Donor and Acceptor Moieties)

The luminescent compounds of the invention can be used with a wide range of energy donor and acceptor molecules to construct luminescence energy transfer pairs, e.g., fluorescence energy transfer (FET) probes. Fluorophores useful in conjunction with the complexes of the invention are known to those of skill in the art. See, for example, Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995).

There is practical guidance available in the literature for selecting appropriate donor-acceptor pairs for particular probes, as exemplified by the following references: Pesce et al., Eds., FLUORESCENCE SPECTROSCOPY (Marcel Dekker, New York, 1971); White et al., FLUORESCENCE ANALYSIS: A PRACTICAL APPROACH (Marcel Dekker, New York, 1970). The literature also includes references providing exhaustive lists of fluorescent and chromogenic molecules and their relevant optical properties, for choosing reporter-quencher pairs (see, for example, Berlman, HANDBOOK OF FLUORESCENCE SPECTRA OF AROMATIC MOLECULES, 2nd Edition (Academic Press, New York, 1971); Griffiths, COLOUR AND CONSTITUTION OF ORGANIC MOLECULES (Academic Press, New York, 1976); Bishop, Ed., INDICATORS (Pergamon Press, Oxford, 1972); Haugland, HANDBOOK OF FLUORESCENT PROBES AND RESEARCH CHEMICALS (Molecular Probes, Eugene, 1992) Pringsheim, FLUORESCENCE AND PHOSPHORESCENCE (Interscience Publishers, New York, 1949); and the like. Further, there is extensive guidance in the literature for derivatizing reporter and quencher molecules for covalent attachment via readily available reactive groups that can be added to a molecule.

The diversity and utility of chemistries available for conjugating fluorophores to other molecules and surfaces is exemplified by the extensive body of literature on preparing nucleic acids derivatized with fluorophores. See, for example, Haugland (supra); Ullman et al., U.S. Pat. No. 3,996,345; Khanna et al., U.S. Pat. No. 4,351,760. Thus, it is well within the abilities of those of skill in the art to choose an energy exchange pair for a particular application and to conjugate the members of this pair to a probe molecule, such as, for example, a small molecular bioactive material, nucleic acid, peptide or other polymer.

In a FRET pair, it is generally preferred that an absorbance band of the acceptor substantially overlap a fluorescence emission band of the donor. When the donor (fluorophore) is a component of a probe that utilizes fluorescence resonance energy transfer (FRET), the donor fluorescent moiety and the quencher (acceptor) of the invention are preferably selected so that the donor and acceptor moieties exhibit fluorescence resonance energy transfer when the donor moiety is excited. One factor to be considered in choosing the fluorophore-quencher pair is the efficiency of fluorescence resonance energy transfer between them. Preferably, the efficiency of FRET between the donor and acceptor moieties is at least 10%, more preferably at least 50% and even more preferably at least 80%. The efficiency of FRET can easily be empirically tested using the methods both described herein and known in the art.

The efficiency of FRET between the donor-acceptor pair can also be adjusted by changing ability of the donor and acceptor to dimerize or closely associate. If the donor and acceptor moieties are known or determined to closely associate, an increase or decrease in association can be promoted by adjusting the length of a linker moiety, or of the probe itself, between the two fluorescent entities. The ability of donor-acceptor pair to associate can be increased or decreased by tuning the hydrophobic or ionic interactions, or the steric repulsions in the probe construct. Thus, intramolecular interactions responsible for the association of the donor-acceptor pair can be enhanced or attenuated. Thus, for example, the association between the donor-acceptor pair can be increased by, for example, utilizing a donor bearing an overall negative charge and an acceptor with an overall positive charge.

In addition to fluorophores that are attached directly to a probe, the fluorophores can also be attached by indirect means. In this embodiment, a ligand molecule (e.g., biotin) is preferably covalently bound to the probe species. The ligand then binds to another molecules (e.g., streptavidin) molecule, which is either inherently detectable or covalently bound to a signal system, such as a fluorescent compound of the invention, or an enzyme that produces a fluorescent compound by conversion of a non-fluorescent compound. Useful enzymes of interest as labels include, for example, hydrolases, particularly phosphatases, esterases and glycosidases, or oxidotases, particularly peroxidases. Fluorescent compounds include fluorescein and its derivatives, rhodamine and its derivatives, dansyl, umbelliferone, etc., as discussed above. For a review of various labeling or signal producing systems that can be used, see, U.S. Pat. No. 4,391,904.

A non-limiting list of exemplary donor or acceptor moieties that can be used in conjunction with the luminescent complexes of the invention, is provided in Table 1.

TABLE 1
Suitable Moieties Useful as Acceptors in FRET Pairs

4-acetamido-4′-isothiocyanatostilbene-2,2′disulfonic acid
acridine and derivatives:
acridine
acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide
anthranilamide
BODIPY
Brilliant Yellow
coumarin and derivatives:
coumarin
7-amino-4-methylcoumarin (AMC, Coumarin 120)
7-amino-4-trifluoromethylcouluarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin and derivatives:
eosin
eosin isothiocyanate
erythrosin and derivatives:
erythrosin B
erythrosin isothiocyanate
ethidium
fluorescein and derivatives:
5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
QFITC (XRITC)
fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene and derivatives:
pyrene
pyrene butyrate
succinimidyl 1-pyrene butyrate
quantum dots
Reactive Red 4 (Cibacron ™ Brilliant Red 3B-A)
rhodamine and derivatives:
6-carboxy-X-rhodamine (ROX)
6-carboxyrhodamine (R6G)
lissamine rhodamine B sulfonyl chloride rhodamine (Rhod)
rhodamine B
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
lanthanide chelate derivatives

Structures of exemplary functionalized fluorophores of use in the compounds of the invention are set forth herein. Similar derivatization strategies for each of the fluorophores set forth in Tables 1-3 are available and applicable to the invention.

Exemplary commercially available acceptors are listed in Table 2 including protein based acceptors and quenchers.

TABLE 2
Exemplary fluorophores

| | Excitation | Emission |
|---|---|---|
| Acceptor Name | (nm) | (nm) |
| Fluorescein (FITC, FAM) | 494 | 518 |
| Eosin | 524 | |
| TRITC | 543 | |
| TET | 525 | 540 |
| HEX; JOE; VIC; CAL Fluor Orange 560 | 535 | 555 |
| ROX (5/6-carboxy Rhodamine); LC Red | 575 | 605 |
| 610; Cal Fluor Red 610 | | |
| Rhodamine 101 | 496 | 520 |
| Rhodamine Red | 570 | |
| Texas Red | 595 | 615 |
| Texas Red; LC Red 610; CAL Fluor | 590 | 610 |
| Red 610 | | |
| Cy2 | 489 | 506 |
| Cy3 | 548 | 562 |
| Cy3; NED; Quasar 570; Oyster 556 | 550 | 570 |
| Cy5; LC Red 670; Quasar 670; Oyster 645 | 649 | 670 |
| Malachite Green | 630 | |
| Tetramethyl Rhodamine (TAMRA, TMR, | 555 | 580 |
| TRITC) | | |
| Acridine orange | 500 | 530 |
| Bodipy 530/550 | 534 | 554 |
| BODIPY TR-X | 588 | 616 |
| GFP | 489 | 509 |
| LC Red 640; Cal Fluor Red 635 | 625 | 640 |
| Nile Red | 485 | 525 |
| Oregon Green 488 | 493 | 520 |
| YOYO-1 | 491 | 509 |
| YOYO-2 | 612 | 631 |
| Ca-Green | 506 | 534 |
| Ca-Orange | 555 | 576 |
| Ca-Crimson | 588 | 610 |
| Mg-Green | 506 | 532 |
| Na-Green | 507 | 532 |
| Oxonol V | 610 | 639 |
| PROTEIN FLUOROPHORES | | |
| EGFP | 489 | 508 |
| dsRED | 558 | 583 |
| B-Phycoerythrin | 546, 565 | 575 |
| R-Phycoerythrin | 480, 546, 565 | 578 |
| allophycocyanin | 650 | 660 |

| FRET QUENCHERS | | |
|---|---|---|
| Quencher Name | ε (cm⁻¹M⁻¹) | Absorption Max (nm) |
| QSY 7 | 90,000 | 570 |
| QSY-9 | 88,000 | 562 |
| QSY-35 | 23,000 | 475 |
| BHQ-1 | | 535 |
| BHQ-2 | | 580 |
| DDQ-I | | 430 |
| Dabcyl | | 475 |
| Eclipse | | 530 |
| Iowa Black FQ | | 532 |
| DDQ-II | | 630 |
| Iowa Black RQ | | 645 |

In one embodiment, the fluorophore is a member of the Alexa Fluor family, such as those set forth in Table 3.

TABLE 3
Alexa Fluor ® as Exemplary Acceptor
Fluorophores for 4-Tb Donor.

| | | Ex | Em | MW | |
|---|---|---|---|---|---|
| | Color¹ | (nm) | (nm) | (g/mol) | ε (cm⁻¹M⁻¹) |
| Alexa Fluor 350 | blue | 346 | 442 | 410 | 19,000 |
| Alexa Fluor 405 | violet | 401 | 421 | 1028 | 34,000 |
| Alexa Fluor 430 | green | 434 | 541 | 702 | 16,000 |
| Alexa Fluor 488 | green | 495 | 519 | 643 | 71,000 |
| Alexa Fluor 500 | green | 502 | 525 | 700 | 71,000 |
| Alexa Fluor 514 | green | 517 | 542 | 714 | 80,000 |
| Alexa Fluor 532 | green | 532 | 554 | 721 | 81,000 |
| Alexa Fluor 546 | yellow-green | 556 | 573 | 1079 | 104,000 |
| Alexa Fluor 555 | green | 555 | 565 | ~1250 | 150,000 |
| Alexa Fluor 568 | orange | 578 | 603 | 792 | 91,300 |
| Alexa Fluor 594 | orange-red | 590 | 617 | 820 | 90,000 |
| Alexa Fluor 610 | red | 612 | 628 | 1172 | 138,000 |
| Alexa Fluor 633 | not vis | 632 | 647 | ~1200 | 100,000 |
| Alexa Fluor 647 | not vis | 650 | 665 | ~1300 | 239,000 |
| Alexa Fluor 660 | not vis | 663 | 690 | ~1100 | 132,000 |
| Alexa Fluor 680 | not vis | 679 | 702 | ~1150 | 184,000 |
| Alexa Fluor 700 | not vis | 702 | 723 | ~1400 | 192,000 |
| Alexa Fluor 750 | not vis | 749 | 775 | ~1300 | 240,000 |

¹Approximate color of the emission spectrum.
ε = extinction coefficient

Presently preferred Alexa Fluor fluorophores include 488, 500, 532, 546, 555, 568, 594, 610 and 633.

US/2008/0213780, incorporated by reference, discloses nucleic acid probes, methods of their use and other methods that may be applicable to the compounds and methods of the present invention.

Means of detecting fluorescent labels are well known to those of skill in the art. Thus, for example, fluorescent labels can be detected by exciting the fluorophore with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product.

### Methods

The compounds and complexes of the invention are useful as probes in a variety of biological assay systems and diagnostic applications. An overview of assay systems, such as competitive assay formats, immunological assays, microarrays, membrane binding assays and enzyme activity assays, is given e.g., in U.S. Pat. No. 6,864,103 to Raymond et al., which is incorporated herein in its entirety for all purposes. It is within the ability of one of skill in the art to select and prepare a probe that includes a complex of the invention, and which is suitable for each assay system. In an exemplary embodiment, the luminescent probe molecule is used to detect the presence or absence of an analyte in a sample.

In an exemplary embodiment, the complex according to Formula I is utilized in a procedure wherein emission from the complex excites at least one fluorophore in an assay. In another exemplary embodiment, emission from the complex excites at least two fluorophores in an assay, such that each fluorophore emits light of a characteristic wavelength and lifetime. In this example, each of the at least two fluorophores is distinguishable from the other on the basis of emission wavelength and/or lifetime. See, for example, Chen et al., J. Am. Chem. Soc., 122: 657-660 (2000). In a preferred embodiment, the complex of the invention distinguishably excites at least 2, 3, 4, 5, 6, 7, 8, 9 or at least 10 fluorophores essentially simultaneously.

In one aspect, the invention provides a method of detecting a nucleotide, the method comprising: (a) forming a complex between a nucleic acid and a DNA polymerase comprising a first luminescent group, wherein the nucleic acid comprises a template strand and a primer hybridized to the template strand; (b) extending the primer with the nucleotide by contacting the complex with the nucleotide, wherein the nucleotide is a dNTP comprising a second luminescent group; (c) exciting the complex with light, whereby energy is transferred between the first luminescent group and the second luminescent group; and (d) detecting energy emitted by the complex, thereby detecting the nucleotide.

In some embodiments, the DNA polymerase is bound to a solid support. A “polymerase” or “DNA polymerase”, as used herein, refers to any protein that is useful for covalently binding a free nucleotide to an oligonucleotide (known as a primer). The primer is typically complementary and bound to a template oligonucleotide. A polymerase will bind a free nucleotide to the 3′ terminus of the primer, the free nucleotide being complementary to the template nucleotide immediately following the 3′ terminus of the primer. Many polymerases are known in the art. An exemplary polymerase can be T4 polymerase or Taq DNA polymerase. A “solid support” refers to any material appropriate for immobilizing a molecule, such as a polymerase or capture probe, in solution. Suitable substrates include metal surfaces such as gold, electrodes, glass and modified or functionalized glass, plastics (including acrylics, polystyrene and copolymers of styrene and other materials, polypropylene, polyethylene, polybutylene, polycarbonate, polyurethanes, Teflon, derivatives thereof, etc.), polysaccharides, nylon or nitrocellulose, resins, mica, silica or silica-based materials including silicon and modified silicon, carbon, metals, inorganic glasses, fiberglass, ceramics, GETEK (a blend of polypropylene oxide and fiberglass) and a variety of other polymers.

In some embodiments, the first luminescent group is an energy transfer donor.

In some embodiments, the energy transfer donor has a structure according to the formula:

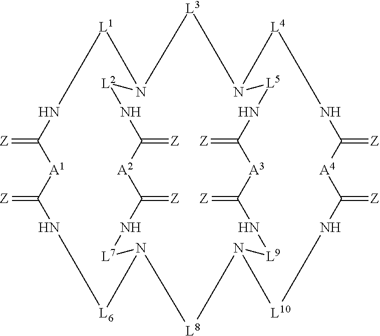

wherein each Z is a member independently selected from O and S; L¹, L², L³, L⁴, L⁵, L⁶, L⁷, L⁸, L⁹ and L¹⁰ are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; A¹, A², A³ and A⁴ are members independently selected from the general structure:

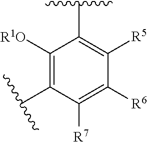

wherein each R¹ is a member independently selected from H, an enzymatically cleavable group, a hydrolytically cleavable group, a metabolically cleavable group and a single negative charge; and each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and NO₂; wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl; and R¹⁷ and R¹⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R¹⁷ and R¹⁸, together with the atoms to which they are attached, are optionally joined to form a 5- to 7-membered ring; wherein the energy transfer donor comprises a linkage to the DNA polymerase. In some embodiments, R¹ is H. In some embodiments, R¹ is not present; i.e., R¹ is nil. In some embodiments, R⁵, R⁶ and R⁷ are selected from H, halogen, alkyl, haloalkyl, heteroalkyl, aryl, heteroaryl, —C(O)R¹⁷, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, wherein R¹⁷ and R¹⁸ are selected from H and alkyl. In some embodiments, R⁵, R⁶ and R⁷ are H.

In some embodiments, the energy transfer donor has the structure:

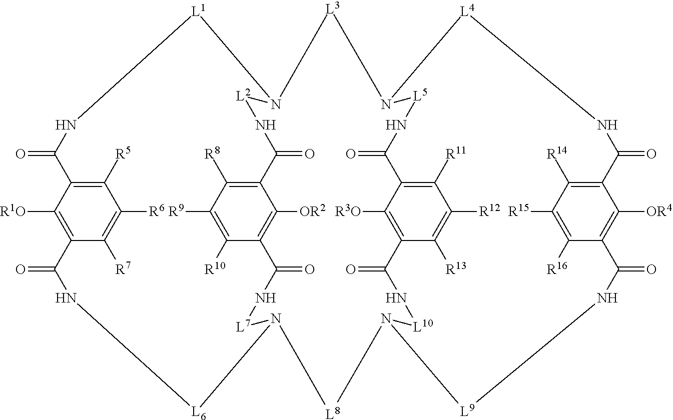

wherein R², R³ and R⁴ have the same definition as R¹; and R⁸, R⁹ and R¹⁰ have the same definition as R⁵, R⁶ and R⁷, respectively; R¹¹, R¹² and R¹³ have the same definition as R⁵, R⁶ and R⁷, respectively; and R¹⁴, R¹⁵ and R¹⁶ have the same definition as R⁵, R⁶ and R⁷, respectively.

In some embodiments, the energy transfer donor has a structure selected from:

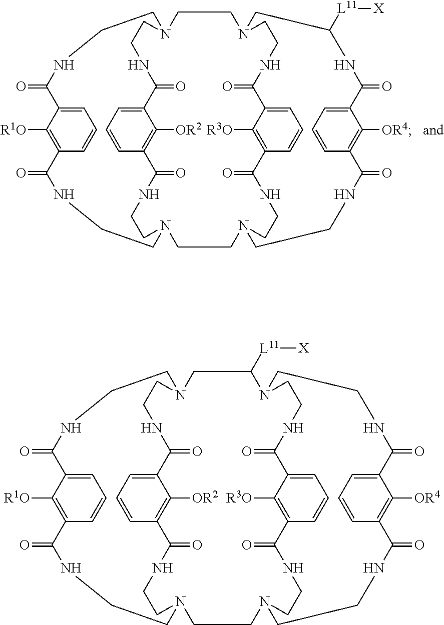

wherein L¹¹ is a member selected from a bond, acyl, substituted or unsubstituted alkyl, substituted or unsubstituted heteroalkyl, substituted or unsubstituted aryl, substituted or unsubstituted heteroaryl, substituted or unsubstituted heterocycloalkyl and nucleic acid; and X is a linkage fragment covalently binding the DNA polymerase to L¹¹.

In some embodiments, the linker moieties L¹, L², L³, L⁴, L⁵, L⁶, L⁷, L⁸, L⁹ and L¹⁰ are members independently selected from substituted or unsubstituted C₁ to C₆ alkyl. In some embodiments, the linker moieties L¹, L², L³, L⁴, L⁵, L⁶, L⁷, L⁸, L⁹ and L¹⁰ are members independently selected from substituted or unsubstituted ethyl. In some embodiments, at least one of L¹, L², L³, L⁴, L⁵, L⁶, L⁷, L⁸, L⁹ and L¹⁰ comprises a linkage to the DNA polymerase.

In some embodiments, the dNTP is not blocked at the 3′ position. In some embodiments, the dNTP is blocked at the 3′ position (for example, by a 3′ block). A dNTP that is blocked (and sometimes referred to herein as “a blocked dNTP”) will lack the ability to be extended by a DNA polymerase. Any number of chemical groups known in the art can be used as a 3′ block. In some embodiments, a 3′ block is —R′-M wherein R′ is alkyl and M is a polar group. In particular embodiments, R′ is an alkyl selected from C₁, C₂, C₃, C₄, C₅, C₆, C₇, C₈, C₉, C₁₀ alkyl. In some embodiments, M is selected from —OR″, —SH, —SR″, —NH₂, —NHR″, —NR″₂ and —CN, wherein R″ is alkyl, which in some embodiments is an alkyl selected from C₁, C₂, C₃, C₄, C₅ and C₆ alkyl. In some embodiments, the 3′ block is selected from —(CH₂)₃SH, —(CH₂)₃NH₂, —(CH₂)₃OH and inorganic phosphate. In particular embodiments, the 3′ block is —(CH₂)₃OH. In some embodiments, the method further comprises removing the 3′ block (i.e., unblocking the blocked dNTP). In some embodiments, the method further comprises removing the second luminescent group. In some embodiments, the method further comprises repeating the method to add a further dNTP for identifying the next nucleotide in the template strand.

In some embodiments, the dNTP is covalently linked at the 3′ position to a cleavable group. In some embodiments, the cleavable group is selected from a hydrolytically cleavable group, an enzymatically cleavable group and a photolytically cleavable group. In some embodiments, the cleavable group is a photolytically cleavable group.

In some embodiments, the photolytically cleavable group comprises

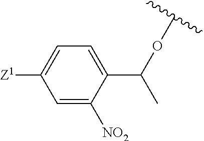

wherein Z¹ is the second luminescent group.

In some embodiments, a method herein comprises exposing the complex to UV light.

In some embodiments, the second luminescent group is a fluorophore acceptor.

In some embodiments, one of the luminescent groups is chelated to a metal ion.

In some embodiments, the metal ion is a lanthanide ion.

In some embodiments, the lanthanide is a selected from neodynium (Nd), samarium (Sm), europium (Eu), terbium (Tb), dysprosium (Dy) and ytterbium (Yb).

In one aspect, the invention provides a method of detecting a nucleotide, the method comprising: (a) forming a complex between a nucleic acid and a capture probe, wherein the capture probe is bound to a solid support; (b) contacting the complex with a DNA polymerase and the nucleotide, wherein the nucleotide is a blocked dNTP comprising a first luminescent group and a second luminescent group, thereby extending the nucleic acid with the nucleotide; (c) washing the solid support; (d) exciting the complex with light, whereby energy is transferred between the first luminescent group and the second luminescent group; and (e) detecting energy emitted by the second luminescent group, thereby detecting the nucleotide. In some embodiments, the method further comprises removing the first and second luminescent groups. In some embodiments, the method further comprises unblocking the blocked dNTP. In some embodiments, the method further comprises contacting the complex with a second blocked dNTP comprising a first luminescent group and a second luminescent group, thereby extending the nucleic acid with the second blocked dNTP. In some embodiments, the method comprises repeating steps (c) to (e).

In some embodiments, the first luminescent group is an energy transfer donor, the second luminescent group is an energy transfer acceptor, the donor and acceptor are covalently joined to form a donor-acceptor assembly, and the donor-acceptor assembly is joined to a dNTP by a donor-acceptor linker comprising a cleavable group.

In some embodiments, the first luminescent group is an energy transfer donor, the second luminescent group is an energy transfer acceptor, and the donor and acceptor are joined by a donor-acceptor linker comprising a cleavable group.

In some embodiments, the energy transfer donor has a structure according to the formula:

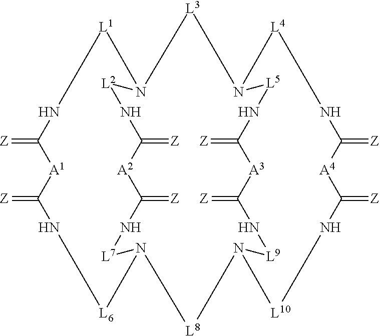

wherein each Z is a member independently selected from O and S; L¹, L², L³, L⁴, L⁵, L⁶, L⁷, L⁸, L⁹ and L¹⁰ are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; A¹, A², A³ and A⁴ are members independently selected from the general structure:

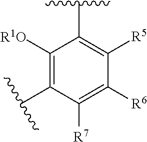

wherein each R¹ is a member independently selected from H, an enzymatically cleavable group, a hydrolytically cleavable group, a metabolically cleavable group and a single negative charge; and each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and NO₂; wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl; and R¹⁷ and R¹⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R¹⁷ and R¹⁸, together with the atoms to which they are attached, are optionally joined to form a 5- to 7-membered ring, wherein the energy transfer donor comprises a linkage to the donor-acceptor linker. In some embodiments, R¹ is H. In some embodiments, R¹ is not present; i.e., R¹ is nil. In some embodiments, R⁵, R⁶ and R⁷ are selected from H, halogen, alkyl, haloalkyl, heteroalkyl, aryl, heteroaryl, —C(O)R¹⁷, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, wherein R¹⁷ and R¹⁸ are selected from H and alkyl. In some embodiments, R⁵, R⁶ and R⁷ are H.

In some embodiments, the energy transfer donor has the structure:

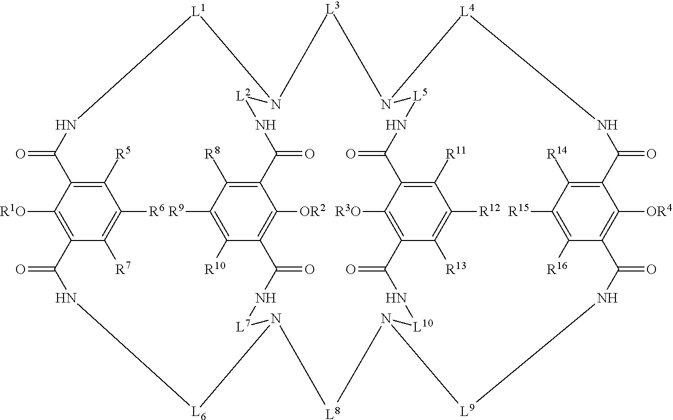

wherein R², R³ and R⁴ have the same definition as R¹; and R⁸, R⁹ and R¹⁰ have the same definition as R⁵, R⁶ and R⁷, respectively; R¹¹, R¹² and R¹³ have the same definition as R⁵, R⁶ and R⁷, respectively; and R¹⁴, R¹⁵ and R¹⁶ have the same definition as R⁵, R⁶ and R⁷, respectively.

In some embodiments, the energy transfer donor has a structure selected from:

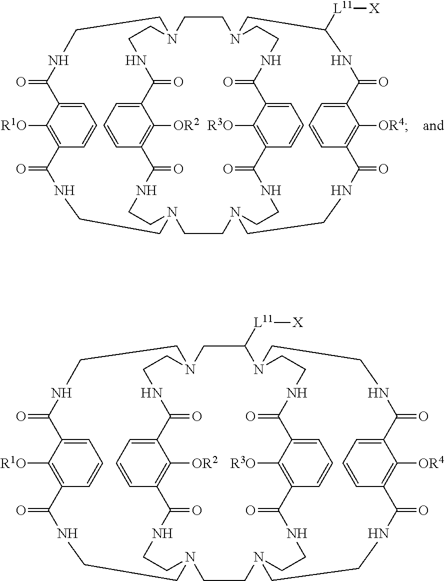

wherein L¹¹ is a member selected from a bond, acyl, substituted or unsubstituted alkyl, substituted or unsubstituted heteroalkyl, substituted or unsubstituted aryl, substituted or unsubstituted heteroaryl, substituted or unsubstituted heterocycloalkyl and nucleic acid; and X is a linkage fragment covalently binding the donor-acceptor linker to L¹¹.

In some embodiments, the linker moieties L¹, L², L³, L⁴, L⁵, L⁶, L⁷, L⁸, L⁹ and L¹⁰ are members independently selected from substituted or unsubstituted C₁ to C₆ alkyl. In some embodiments, the linker moieties L¹, L², L³, L⁴, L⁵, L⁶, L⁷, L⁸, L⁹ and L¹⁰ are members independently selected from substituted or unsubstituted ethyl. In some embodiments, at least one of L¹, L², L³, L⁴, L⁵, L⁶, L⁷, L⁸, L⁹ and L¹⁰ comprises a linkage to the linker between the first luminescent group and the second luminescent group.

In some embodiments, the donor-acceptor linker comprises substituted or unsubstituted aryl or substituted or unsubstituted heteroaryl.

In some embodiments, the donor-acceptor linker comprises

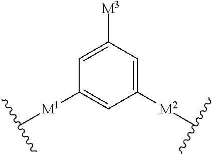

wherein M¹ and M² are independently selected from substituted or unsubstituted alkyl and substituted or unsubstituted heteroalkyl; and M³ is a linker to DNA and comprises a cleavable group.

In some embodiments, the cleavable group is selected from a hydrolytically cleavable group, an enzymatically cleavable group and a photolytically cleavable group.

In some embodiments, the cleavable group comprises

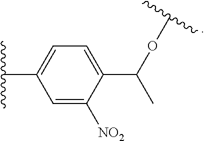

In some embodiments, the energy transfer acceptor is a fluorophore acceptor.

In some embodiments, one of the luminescent groups is chelated to a metal ion.

In some embodiments, the metal ion is a lanthanide ion.

In some embodiments, the lanthanide is a selected from neodynium (Nd), samarium (Sm), europium (Eu), terbium (Tb), dysprosium (Dy) and ytterbium (Yb).

In one aspect, the invention provides a compound having the structure: Q¹-G, wherein Q¹ is a luminescent group and G is a cleavable group.

In some embodiments, Q¹ is an energy transfer acceptor.

In some embodiments, Q¹ is a fluorophore acceptor.

In some embodiments, G is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

In some embodiments, G is substituted or unsubstituted aryl.

In some embodiments, G comprises

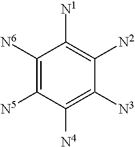

wherein N¹, N², N³, N⁴, N⁵ and N⁶ are independently selected from H, halogen, haloalkyl, —NO₂, —CN, —SO₃H, —CO₂H, —CHO, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and one of N¹, N², N³, N⁴, N⁵ and N⁶ is bonded to Q¹.

In some embodiments, a compound has the structure

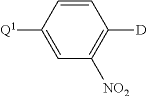

wherein D is selected from —C(O)CH₃, —C(O)(0)CH₃ and a linkage to a nucleotide.

In one aspect, the invention provides a compound having the structure: Q²-G wherein Q² comprises a first luminescent group and a second luminescent group; and G is a cleavable group; wherein the first luminescent group is an energy transfer donor and the second luminescent group is an energy transfer acceptor.

In some embodiments, G is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

In some embodiments, G is substituted or unsubstituted aryl.

In some embodiments, G comprises

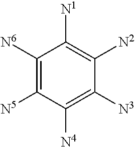

wherein N¹, N², N³, N⁴, N⁵ and N⁶ are independently selected from H, halogen, haloalkyl, —NO₂, —CN, —SO₃H, —CO₂H, —CHO, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and one of N¹, N², N³, N⁴, N⁵ and N⁶ is bonded to Q².

In some embodiments, a compound has the structure

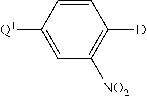

wherein D is selected from —C(O)CH₃, —C(O)(0)CH₃ and a linkage to a nucleotide.

In some embodiments, the energy transfer acceptor is a fluorophore acceptor.

In some embodiments, the energy transfer donor has a structure according to the formula:

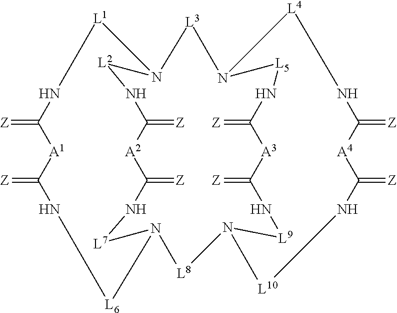

wherein each Z is a member independently selected from O and S; L¹, L², L³, L⁴, L⁵, L⁶, L⁷, L⁸, L⁹ and L¹⁰ are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; A¹, A², A³ and A⁴ are members independently selected from the general structure:

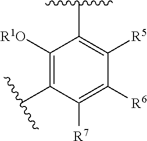

wherein each R¹ is a member independently selected from H, an enzymatically cleavable group, a hydrolytically cleavable group, a metabolically cleavable group and a single negative charge; and each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and —NO₂, wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl; and R¹⁷ and R¹⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R¹⁷ and R¹⁸, together with the atoms to which they are attached, are optionally joined to form a 5- to 7-membered ring. In some embodiments, R¹ is H. In some embodiments, R¹ is not present; i.e., R¹ is nil. In some embodiments, R⁵, R⁶ and R⁷ are selected from H, halogen, alkyl, haloalkyl, heteroalkyl, aryl, heteroaryl, —C(O)R¹⁷, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, wherein R¹⁷ and R¹⁸ are selected from H and alkyl. In some embodiments, R⁵, R⁶ and R⁷ are H.

In some embodiments, Q² has the structure

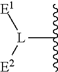

wherein E¹ is the energy transfer donor, E² is the energy transfer acceptor and L is a linker selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

In some embodiments, Q² has the structure

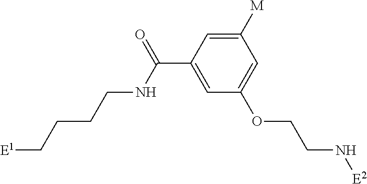

wherein M is a linkage to G.

In some embodiments, one of the luminescent groups is chelated to a metal ion.

In some embodiments, the metal ion is a lanthanide ion.

In some embodiments, the lanthanide is a selected from neodynium (Nd), samarium (Sm), europium (Eu), terbium (Tb), dysprosium (Dy) and ytterbium (Yb).

In some embodiments, the energy transfer acceptor is a compound disclosed herein.

In some embodiments, the energy transfer donor is a compound disclosed herein.

In some embodiments, the first luminescent group or the second luminescent group is a compound disclosed herein.

The disclosed lanthanide complexes have particular utility in assays that are intended to detect or quantify binding or other modification of an assay component. These assays may incorporate one or more steps, including (a) contacting at least one member of a plurality of molecules with a binding partner capable of binding one of the molecules, (b) detecting a response indicative of the extent of binding between the at least one member of the plurality and the binding partner, and (c) correlating the response with the extent of binding or modification, or with the activity of an enzyme that affects the modification. In some embodiments, the assays may include repeating the steps of contacting, detecting, and/or correlating for the same sample and/or a plurality of different samples. The assays may also involve providing a sample holder having a plurality of sample sites containing or supporting a corresponding plurality of samples, and sequentially and/or simultaneously repeating the steps of contacting, detecting, and/or correlating for the plurality of samples. The remainder of this section describes in more detail the steps of (a) contacting, (b) detecting, and (c) correlating.

The step of contacting assay components such as binding partners (e.g., nucleic acids, peptides, enzymes, enzyme modulators, substrates, products) with one another and/or with other species generally comprises any method for bringing any specified combination of these components into functional and/or reactive contact. A preferred method is by mixing and/or forming the materials in solution, although other methods, such as attaching one or more components (e.g., a complex according to Formula I, a species comprising a complex according to Formula I or other assay component) to a bead or surface, also may be used, as long as the components retain at least some function, specificity, and/or binding affinity following such attachment. The assay may be carried out in a device for manipulating fluids. Useful assay apparati having fluidics capability (e.g., microfluidics) suitable for contacting or otherwise preparing assay components are generally known in the art.

One or more of the assay components may comprise a sample, which typically takes the form of a solution containing one or more analyte that are biological and/or synthetic in origin. The sample may be a biological sample that is prepared from a blood sample, a urine sample, a swipe, or a smear, among others. Alternatively, the sample may be an environmental sample that is prepared from an air sample, a water sample, or a soil sample, among others. The sample typically is aqueous but may contain compatible organic solvents, buffering agents, inorganic salts, and/or other components known in the art for assay solutions.

The assay components and/or sample may be supported for contact and/or detection and/or analysis by any substrate or material capable of providing such support. Suitable substrates may include microplates, PCR plates, biochips, and hybridization chambers, among others, where features such as microplate wells and microarray (i.e., biochip) sites may comprise assay sites. Microplates may include 96, 384, 1536, or other numbers of wells. These microplates also may include wells having small (≈50 μL) volumes, elevated bottoms, and/or frusto-conical shapes capable of matching a sensed volume. Suitable PCR plates may include the same (or a similar) footprint, well spacing, and well shape as the preferred microplates, while possessing stiffness adequate for automated handling and thermal stability adequate for PCR. Suitable microarrays include nucleic acid and polypeptide microarrays, which are generally known in the art.

The step of detecting a response indicative of the extent of binding or modification generally comprises any method for effectuating such detection, including detecting and/or quantifying a change in, or an occurrence of, a suitable parameter and/or signal. The method may include luminescence and/or nonluminescence methods, and heterogeneous and/or homogeneous methods, among others.

Luminescence and nonluminescence methods may be distinguished by whether they involve detection of light emitted by a component of the sample. Luminescence assays involve detecting light emitted by a luminescent compound (or luminophore) and using properties of that light to understand properties of the compound and its environment. A typical luminescence assay may involve (1) exposing a sample to a condition capable of inducing luminescence from the sample, and (2) measuring a detectable luminescence response indicative of the extent of binding between the member of interest and a corresponding binding partner. Suitable luminescence assays include, among others, (1) luminescence intensity, which involves detection of the intensity of luminescence, (2) luminescence polarization, which involves detection of the polarization of light emitted in response to excitation by polarized light, (3) luminescence energy transfer, and (4) luminescence lifetime. A single assay mixture may be analyzed by one or more of these techniques. In a preferred embodiment, energy exchange between a luminescent complex of the invention and a fluorophore is utilized to detect the analyte (and optionally its degree of modification or binding to a binding partner) is utilized to determine both the emission wavelength and excitation lifetime of one or more fluorophores.

The detectable luminescence response generally comprises a change in, or an occurrence of, a luminescence signal that is detectable by direct visual observation and/or by suitable instrumentation. Typically, the detectable response is a change in a property of the luminescence, such as a change in the intensity, polarization, energy transfer, lifetime, and/or excitation or emission wavelength distribution of the luminescence. For example, energy transfer may be measured as a decrease in donor luminescence, an increase (often from zero) in acceptor luminescence, and/or a decrease in donor luminescence lifetime, among others. The detectable response may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence assays, the detectable response may be generated directly using a donor or acceptor associated with an assay component actually involved in binding, or indirectly using a donor or acceptor associated with another (e.g., reporter or indicator) component. Suitable methods and donors and acceptors for luminescently labeling assay components are described in the following materials, which are incorporated herein by reference: Richard P. Haugland, Handbook of Fluorescent Probes and Research Chemicals (6th ed. 1996).

Heterogeneous and homogeneous methods may be distinguished by whether they involve sample separation before detection. Heterogeneous methods generally require bulk separation of bound and unbound species. This separation may be accomplished, for example, by washing away any unbound species following capture of the bound species on a solid phase, such as a bead or microplate surface labeled with a trivalent metal or other suitable binding partner. Such metals may include gallium (Ga, including Ga(III)), iron (Fe), aluminum (Al), and/or zinc (Zn), among others. Suitable metals and other binding partners are described in more detail in U.S. patent application Ser. No. 10/746,797, filed Dec. 23, 2003, which is incorporated herein by reference. The extent of binding then can be determined directly by measuring the amount of captured bound species and/or indirectly by measuring the amount of uncaptured unbound species (if the total amount is known). Homogeneous methods, in contrast, generally do not require bulk separation but instead require a detectable response such as a luminescence response that is affected in some way by binding or unbinding of bound and unbound species without separating the bound and unbound species. Alternatively, or in addition, enzyme activity may result in increased or decreased energy transfer between a donor and acceptor of an energy transfer pair, based on whether the acceptor quenches or not, and based on whether enzyme activity in the assay results in increased or decreased proximity of the donor and acceptor. Homogeneous assays typically are simpler to perform but more complicated to develop than heterogeneous assays.

The step of correlating generally comprises any method for correlating the extent of binding with the extent of modification of the assay component being analyzed, and/or with the presence and/or activity of an enzyme that affects the modification. The nature of this step depends in part on whether the detectable response is simply detected or whether it is quantified. If the response is simply detected, it typically will be used to evaluate the presence of a component such as a substrate, product, and/or enzyme, or the presence of an activity such as an enzyme or modulator activity. In contrast, if the response is quantified, it typically will be used to evaluate the presence and/or quantity of a component such as a substrate, product, and/or enzyme, or the presence and/or activity of a component such as an enzyme or modulator.

The correlation generally may be performed by comparing the presence and/or magnitude of the response to another response (e.g., derived from a similar measurement of the same sample at a different time and/or another sample at any time) and/or a calibration standard (e.g., derived from a calibration curve, a calculation of an expected response, and/or a luminescent reference material). Thus, for example, in a energy transfer assay for cyclic nucleotide concentration, the cyclic nucleotide concentration in an unknown sample may be determined by matching the energy transfer efficiency measured for the unknown with the cyclic nucleotide concentration corresponding to that efficiency in a calibration curve generated under similar conditions by measuring energy transfer efficiency as a function of cyclic nucleotide concentration.

Thus, in one aspect, the invention provides a mixture of a complex of the invention and an analyte.

In another aspect, the invention provides a method of detecting the presence or absence of an analyte in a sample. The method comprises (a) contacting the sample and a composition including a complex of the invention; (b) exciting the complex; and (c) detecting luminescence from the complex. The presence or absence of the analyte can be indicated by the absence or presence of luminescence from the complex.

In a further aspect, the invention provides a method of detecting the presence or absence of an analyte in a sample. The method comprises (a) contacting the sample and a composition comprising a complex of the invention, and a luminescence modifying group, wherein energy can be transferred between the complex and the luminescence modifying group when the complex is excited, and wherein the complex and the luminescence modifying group can be part of the same molecule or be part of different molecules; and (b) exciting said complex; and (c) determining the luminescent property of the sample, wherein the presence or absence of the analyte is indicated by the luminescent property of the sample.

In diagnostic or prognostic detection methods the subject nucleic acid can comprise a genetic point mutation, deletion, or insertion relative to a naturally occurring or control nucleic acid. Such screening methods can permit the detection of the subject nucleic acid indicating the presence of a genetically associated disease, such as certain cancers, in the sample. There are many well-known examples of genetic mutations already in the art that are indicative of a disease state. The methods include the detection of nucleic acids comprising K-ras, survivin, p53, p16, DPC4, or BRCA2. Furthermore, the methods can be used to detect the amount of a subject nucleic acid being produced by an organism for purposes other than diagnosis or prognosis of a disease or condition. Resonance energy transfer detections of the present invention can be performed with the assistance of single- or multiple-photon microscopy, time-resolved fluorescence microscopy or fluorescence endoscopy, as detailed below.

In one aspect, the invention provides a kit including a recognition molecule and a compound or a complex of the invention. Exemplary recognition molecules include biomolecules, such as whole cells, cell-membrane preparations, antibodies, antibody fragments, proteins (e.g., cell-surface receptors, such as G-protein coupled receptors), protein domains, peptides, nucleic acids, polymerases and the like.

The invention further provides kits for the detection of a subject nucleic acid comprising the nucleic acid probe compositions described herein, necessary reagents and instructions for practicing the methods of detection. Such alternative compositions, methods and kits therefor are described in more detail by way of the examples, and still others will be apparent to one of skill in the art in view of the present disclosure.

One embodiment of the present invention provides compositions and methods that measure a resonance energy transfer, for example, a fluorescent signal due to FRET or LRET as a result of direct interaction between two molecular beacons when hybridized to the same target nucleic acid of interest. This method can dramatically reduce false-positive signals in gene detection and quantification in living cells. Probe sequences are chosen such that the molecular beacons hybridize adjacent to each other on a single nucleic acid target in a way that positions their respective fluorophores in optimal configuration for FRET. Emission from the acceptor fluorophore serves as a positive signal in the FRET based detection assay.

If acceptor and donor fluorophores are well matched, excitation of the donor can be achieved at a wavelength that has little or no capacity to excite the acceptor; excitation of the acceptor will therefore only occur if both molecular beacons are hybridized to the same target nucleic acid and FRET occurs. Molecular beacons that are degraded or open due to protein interactions will result in the presence of unquenched fluorophore, however, fluorescence emitted from these species is different in character from the signal obtained from donor/acceptor FRET pair, making background and true positive signal more readily differentiated. Thus, by detecting FRET instead of direct single-molecule fluorescence, nucleic acid probe/target binding events can be distinguished from false-positives.

In another embodiment that can be used in the diagnosis or prognosis of a disease or disorder, the target sequence is a naturally occurring or wild type human genomic or RNA or cDNA sequence, mutation of which is implicated in the presence of a human disease or disorder, or alternatively, the target sequence can be the mutated sequence. In such an embodiment, optionally, the amplification reaction can be repeated for the same sample with different sets of probes that amplify, respectively, the naturally occurring sequence or the mutated version. By way of example, the mutation can be an insertion, substitution, and/or deletion of one or more nucleotides, or a translocation.

In a specific embodiment, the compound of the invention is utilized as a component of a high-throughput nucleic acid sequencing application, where 4-color or 2 color fluorophores are a necessity for the current sequencing platforms. In this embodiment, the invention utilizes multiple different acceptor fluorophores, each one excited by a separate emission band of the luminescent complex of the invention. For example, when a Tb chelate is utilized, four different fluorophores can be used as acceptors. An alternative is to use only 3 different acceptor fluorophores and the fourth color is the remaining uncoupled emission peak of the luminescent metal chelate alone.

In an exemplary embodiment, a set of conventional dyes are selected with peak absorption maxima at each of the chelate-Tb emission maxima, identified as A(490 nm), B(545 nm), C(590 nm), and D(620 nm). These conventional dyes are brought into close proximity (e.g., operative proximity) to the chelate-Tb dye for fluorescent resonance energy transfer (FRET) and a number of conceivable scenarios for this are outlined below.

Multicolor dyes based on a luminescent metal chelate donor and various conventional acceptors can be covalently linked (see FIGS.) through synthetic coupling reactions. This has been demonstrated in previous filings as a direct fluorescein conjugate. The obvious extension of this scenario is the inclusion of other acceptors with differing emission wavelengths (see FIGS.). The usefulness of such molecules is enhanced by an additional linkage moiety (e.g., reactive functional group convertible into a linkage fragment) for synthetic attachment of the construct to biologically relevant molecules such as carrier moieties. As shown in the figures, these reactive linkages can originate with the chelate-Tb macrocycle (R1), the synthetic linker (R2), or the conventional fluorophore (R3).

The specific linker chosen for such an application has a direct impact on the function of such a molecule in that the distance between acceptor and donor is directly related to the efficiency of energy transfer impacting both the intensity of acceptor emission and the lifetime. Other physical characteristics such as solubility and stability may also tuned by the specific nature of the linker.

In another exemplary embodiment, a functionally significant variation of the covalent linkage is embodied in the attachment of both donor and acceptor to the same oligo strand, via synthetic coupling schemes. Features include the ability to tailor quite specifically the distance between donor and acceptor as well as the nature of the donor and acceptor. In future application it is envisioned that there might be ‘off the shelf’ luminescent chelates (e.g., Tb chelates) coupled with the 4 standard base pairs (adenine, guanine, cytosine, thymine) with appropriate chemical make-up for use in current or future automated oligo synthesizers. In a preferred embodiment, this provides an assay readable as four different colors, which are produced by excitation at a single wavelength. Accordingly, the present invention provides assays that are readable in at least two, three, four, five, six, seven, or 8 colors (wavelengths), with excitation at a single wavelength.

### Analytes

The compounds, complexes and methods of the invention can be used to detect any analyte or class of analytes in any sample. A sample may contain e.g., a biological fluid (e.g., blood of a patient) or tissue. Other samples can e.g., include solutions of synthetic molecules or extracts from a plant or microorganism (e.g., for drug screening efforts). Exemplary analytes are pharmaceutical drugs, drugs of abuse, synthetic small molecules, biological marker compounds, hormones, infectious agents, toxins, antibodies, proteins, lipids, organic and inorganic ions, carbohydrates and the like. (see, e.g., U.S. Pat. No. 6,864,103 to Raymond et al. for additional examples of analytes).

### Synthesis

The compounds and complexes of the invention are synthesized by an appropriate combination of generally well-known synthetic methods. Techniques useful in synthesizing the compounds of the invention are both readily apparent and accessible to those of skill in the relevant art. US/2008/0213780 provides exemplary methods for synthesizing the compounds disclosed herein.

Lumiphore, Inc. has commercialized the next generation of luminescent lanthanide (Ln³⁺) complexing agent Lumi4®-Tb, based on a 2-hydroxyisophthalamide scaffold [1] that originated at the University of California at Berkeley. This state-of-the-art complexing agent confers exceptional brightness, stability and versatility to terbium fluorescent signals. Research conducted by Lumiphore has shown Lumi4-Tb to have an exceptionally high quantum yield (60%), extinction coefficient (26,000 M⁻¹ cm⁻¹), and long lifetime (2.7 ms) at dilute concentrations in standard aqueous buffer environments. Further, Lumiphore has demonstrated that Lumi4-Tb exhibits long-term stability while maintaining, lifetime, quantum yield and emission spectrum properties following covalent conjugation to a number of different proteins and small molecules.

Lumiphore has demonstrated feasibility of critical stages in developing Lumi4™-Tb into an effective reporter for life science applications. Specifically, 1) the ability to couple Lumi4-Tb donor with organic fluorophore acceptors (L⁴-AC), 2) the effectiveness of using linker modification to enable emission lifetime tuning for a representative acceptor-donor pair, 3) the ability to couple multiple Lumi4-Tb donors to a representative acceptor in an effort to increase total compound fluorescence intensity while maintaining lifetime characteristics, 4) the ability to couple L⁴-AC conjugates to a representative protein (streptavidin) while maintaining photophysical characteristics. These results show that molecules using the Lumi4-Tb lanthanide fluorescence engine, can increase the fluorescence lifetime of acceptor fluorophores in appropriately designed compounds, enabling time-resolved detection at multiple wavelengths. Leveraging this unique characteristic virtually eliminates fluorescent background, improving signal-to-noise ratios and detections limits. This property is expected to improve read length and significantly reduce the cost of whole genome sequencing forming the basis for 4^{th} generation DNA sequencing platforms as well as generally offering the life-science industry a set of multicolored time-resolved reporters.

There are many life science applications, such as high throughput screening (HTS) drug discovery, genomic screening, rapid DNA sequencing, clinical diagnostics and fluorescence microscopy, which require detection of target analytes in low concentration. Typically these applications require many analyses performed in a short period of time without preliminary purification of the sample [2-4]. These applications have led to a significant development of novel reporter molecules and mechanisms over the last two decades. To be useful, probe molecules must have particular properties that allow them to be detected at low concentration in a complex environment. Fluorescent/luminescent probes have been developed as suitable alternatives to radioactively labeled molecules for a variety of applications. Fluorescent/luminescent probes may be selected from fluorescent organic dye molecules, chemiluminescent and chemifluorescent small molecules, the naturally occurring fluorescent proteins such as green fluorescent protein (GFP) and its many genetically engineered relatives, R-phycoerythrin (RPE) and allophycocyanin (APC), luminescent colloidal semiconductor nanocrystals (quantum dots), fluorescent nanoparticles and lanthanide-based luminescent molecules [5-11].

One of the most significant applications is rapid DNA sequencing [12, 13], which plays a critical role in both basic and applied life science research. Fluorescent organic dye molecules have been critical for rapid DNA sequencing (e.g. four-color DNA sequencing technology has been pivotal to the overall success of the Human Genome Project [14]). Developments in fluorescent dyes [15-17] have helped define the current standard for DNA sequencing platforms. However, the core technology of assigning a base-call from the emission wavelength of a dye's fluorescence has not changed since its introduction in 1986 [12] and remains the underlying method used in most instruments [12, 13, 18, 19].

### Limitations of Systems Based on Organic Dye Molecules

Fundamental characteristics of conventional organic fluorescent dyes have limited the sensitivity, speed, and the multiplexed capacity and reliability of current applications. These limitations are caused by the inefficient excitation of fluorescent dyes from a single laser source, significant spectral overlap between the excitation and emission profiles of the dyes, the need to wash away interfering sample matrix, significant loss in fluorescent signal intensities from the required use of a dispersing element or band-pass filters for detection, and complex nature of equipment needed to reliably detect multiple wavelengths in a diagnostic setting. Multi-color systems that rely on small organic fluorophores that absorb and emit over a range of wavelengths are available, but these molecules suffer from broad absorption and emission spectra (which limit the use of multiple dye probes in a single experiment), are susceptible to photobleaching followed repeated excitation, and self-quench when their local concentration is high. In addition, their short fluorescent lifetimes (on the order of ns) correlate with natural fluorescent background found in biological samples, making signal/noise levels unsatisfactory for many applications[20].

These limitations in sensitivity have led to the development of alternative strategies based on non-dispersing techniques such as short fluorescent life-time [21-23] and radio frequency (RF) modulation [24]. However, these new approaches have not had a significant impact on the field of DNA sequencing. New technologies remain a critical need for increased efficiency and cost-effectiveness for high-throughput sequencing [14, 25].

### Potential of Luminescent Lanthanide Metal Complexes

In comparison to the fluorescent organic dye molecules used in DNA sequencing applications, luminescent lanthanide metal complexes have many properties that make them desirable for use [26] in biological assays:
Their long-lived (μs-ms) fluorescence lifetimes allow time-gated detection, leading to especially low detection levels and high signal-noise ratios. Time-gating removes interfering, short-lived auto fluorescence signals of biological samples and containers.
Lanthanide fluorophores have large Stokes shifts (separation between excitation and emission wavelengths) and narrow emission lines prevent the reabsorption of light often seen with conventional organic dyes.
Lanthanide fluorophores have very narrow emission lines, facilitating multiplexing by limiting the overlap between emission spectra.
Lanthanides exhibit exceptionally long Förster distances for transferring energy between molecules by fluorescence resonance energy transfer (FRET).

### Lumiphore's Luminescent Lanthanide Technology.

Lumiphore has developed the next generation of luminescent terbium complexing agent based on a 2-hydroxyisophthalamide scaffold [1]. Although other terbium complexes with higher extinction coefficients and quantum yields have been reported [27], no practical complexes have been described which retain their bright luminescence properties at dilute concentrations in aqueous solutions [28, 29]. The IAM-based structures have high molar extinction coefficients and quantum yields approaching the theoretical maximum in aqueous solutions, and retain all of the benefits of conventional lanthanide reporters (long fluorescence lifetimes, large Stokes shifts, narrow emission lines). For these fluorescent reagents to be useful for biological applications, it is critical that the metal complexes retain the metal ion in aqueous conditions and cause little perturbation of the biomolecule that it is bound to. The acyclic 2-hydroxyisophthalamide complexing agent 1 (A-IAM) forms highly luminescent complexes with Tb³⁺, but studies have indicated that this complex is kinetically labile in certain buffers, which translates to some loss of the metal ion in biological assays (Tb-1). To address this concern, macrocyclic ligand 2 (M-IAM) was prepared and studies have demonstrated that this chelator forms complexes that exhibit much higher kinetic stability than Tb-1. While the unfunctionalized macrocyclic complex is stable under a variety of aqueous conditions, the functionalized macrocyclic complex, Tb-3, exhibits even greater kinetic stability, maintaining its luminescence in the presence of strong competitors and low pH, and has proven useful in biological assays, yielding highly reproducible luminescence at very dilute concentrations.

### Superior Lanthanide Technology

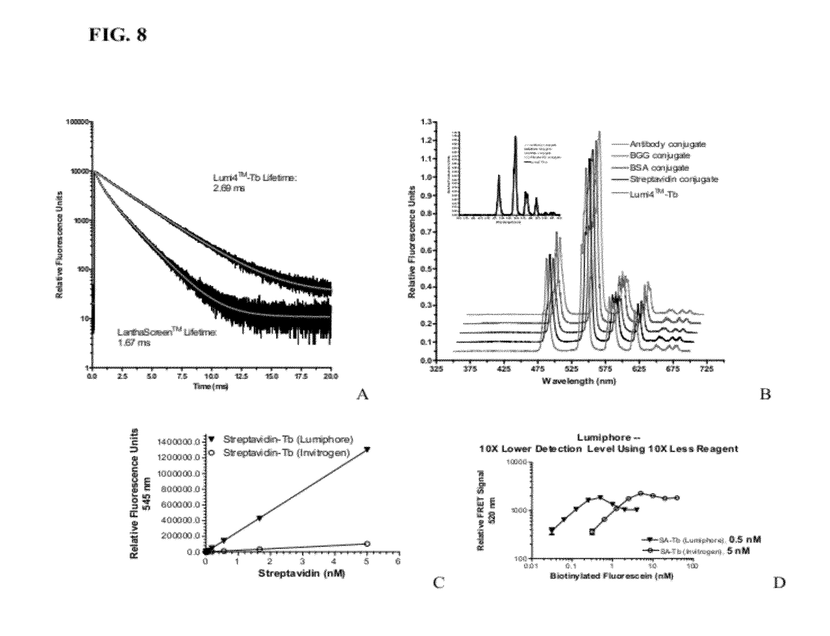

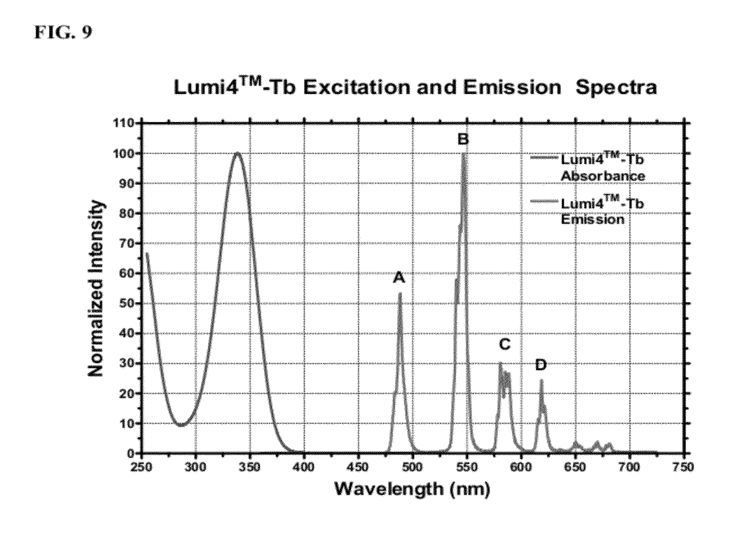

The Lumi4-Tb complex is more than an order of magnitude brighter than any other luminescent lanthanide complex that is currently available commercially and is near the theoretical maximum in quantum efficiency. It is rare for a small molecule fluorophore to achieve a level of brightness that is more than an order of magnitude brighter than any other known fluorophores in the same family. Significant work has occurred over the years in academic laboratories and large commercial companies to produce brighter lanthanide complexes, but these efforts have been unsuccessful. Direct comparisons between Lumiphore's luminescent Lumi4-Tb complex and Invitrogen's Lanthascreen™ technology provide insight into the superior nature of Lumi4-Tb. Lanthascreen complexes are based on a different Tb sensitizing scaffold and are prone to additional quenching when they are conjugated to proteins resulting in a shorter decay half-life and altered emission spectra (FIG. 8-A&B) [30], a complication that is not observed with Lumiphore's technology. Direct comparison in conventional TRF mode (FIG. 8-C) and prototypical TR-FRET experiments (FIG. 8-D) show that Lumi4-Tb is more than an order of magnitude brighter (TRF) and yields 10× lower detection levels (TR-FRET).

This brightness advantage significantly expands the potential value of this technology in the commercial biotechnology marketplace.

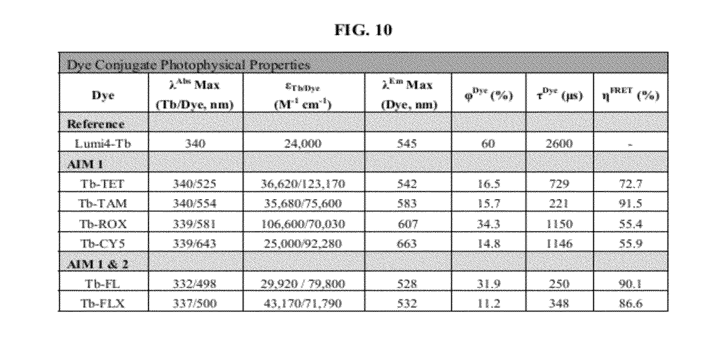

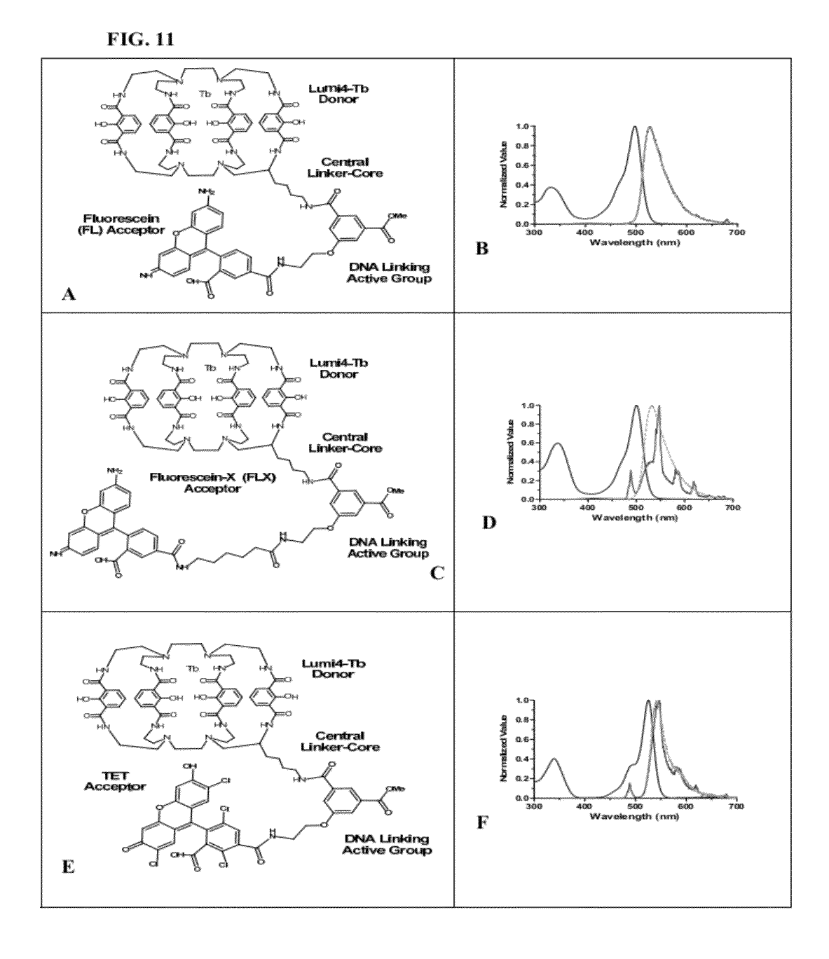

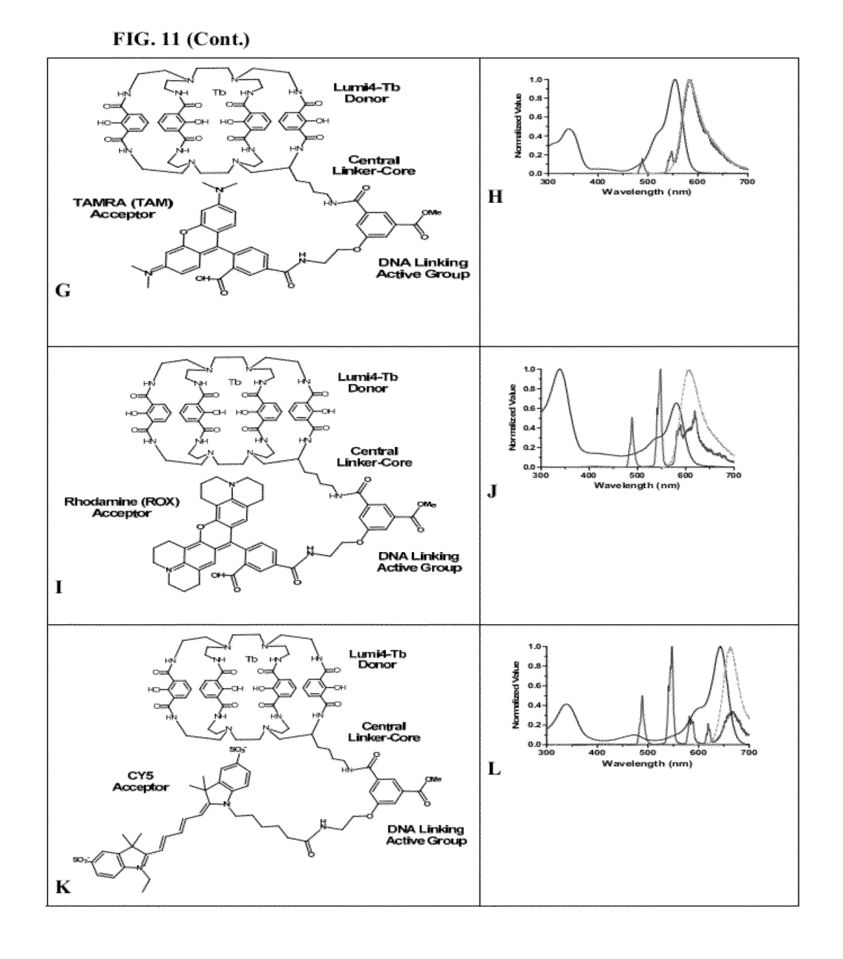

FIG. 9 shows the absorption and emission spectra of Lumi4-Tb. Characteristic of a terbium based fluorophore are the four narrow emission peaks centered at approximately 490 nm, 545 nm, 590 nm, and 620 nm. A set of conventional dyes are selected with peak absorption maxima at each of the Lumi4-Tb emission maxima, identified as A(490 nm), B(545 nm), C(590 nm), and D(620 nm). These conventional dyes can be brought into close proximity to the Lumi4-Tb dye for fluorescent resonance energy transfer (FRET) [31]. Our results indicate that molecules using the lanthanide fluorescence engine, can increase the fluorescence lifetime of acceptor fluorophores in appropriately designed compounds, enabling time-resolved detection. Leveraging this unique characteristic virtually eliminates fluorescent background, improving signal-to-noise ratios and detections limits. This property is expected to improve read length and significantly reduce the cost of whole genome sequencing forming the basis for innovative 4^{th} generation DNA sequencing platforms.

### Novel Real Time Sequencing Technique

The exceptional quantum yield and FRET efficiency results obtained have raised the possibility that sufficient brightness may be obtained to compete with conventional fluorophores without covalent bonding. This presents the reality of a technique for novel real-time sequencing method.

Real-time sequencing has advantages including high throughput and low reagent cost but rely on complex fabrication and various reporting modalities. Current market leaders for 3rd generation sequencers generally rely on expensive and difficult to manufacture substrates that have nanometer sized surface features including pores and wells. These are necessary to interrogate only the single base currently being incorporating into the current strand or passing by the sensing mechanism. Because some promising methods utilizes conventional fluorophores they are fundamentally limited by background fluorescence requiring a mechanism for exclusion of non-incorporating base pairs. Lumiphore's innovative approach overcomes both of these limitations by utilizing FRET mechanism and time resolved fluorescence. Lumiphore compounds' signal intensity rivals that of conventional fluorophores while possessing a long lifetime property. The exceptional transfer efficiency, leads us to propose a conventional FRET sequencing approach, in real-time, that relies on no complex fabrication substrates. No other long lifetime fluorescent donor molecules are bright enough to make this FRET based approach feasible. Innovative advantages include:
1. Single wavelength laser excitation for all bases. Other approaches require either multiple laser excitations (increasing expense and complexity), or rely on poor excitation overlap of a single laser source with some reporters (leading to decreased sensitivity).
2. No complex nanofabrication necessary. Our approach requires a standard slide with surface immobilized polymerase.
3. Leveraging the time domain reduces background to nearly zero, increasing sensitivity. This background reduction can lead to up to 2.5 orders of magnitude increased sensitivity relative to fluorescein.
4. FRET transfer mechanism allows simple exclusion of all bases except the one being incorporated by the polymerase. A simple proximity mechanism triggers the signal generation when a base pair is incorporated.

As this FRET based approach technique is novel but also high risk, we will pursue both it and a more traditional approach.

Lumiphore has covalently linked multiple conventional organic fluorescent dyes to the Lumi4-Tb lanthanide chelate via a central ‘linker-core’ to facilitate coupling to proteins, DNA and other biomolecules. The resultant fluorescent molecules combine the long-lifetime and large Stokes shift benefits of lanthanide fluorophores, with the wide selection of emission maxima available from conventional organic dyes.

Multiple covalent fluorophores were synthesized and characterized. Out of a total of 8 different constructs, 6 were chosen for full characterization and analysis due to their photophysical properties and suitability for this research effort, which are summarized in FIG. 9. The 6 acceptor fluorophores were fluorescein (FL), fluorescein-X (FLX, hexanoic linker), tetrachloro fluoroscein (TET), tetramethyl rhodamine (TAM), rhodamine (ROX) and CY5. All constructs were synthesized with a meta-dibenzoic phenol based linker core. Orthogonal protection allowed the covalent attachment of both the Lumi4-Tb compound to one arm and a variety of acceptor fluorophores to a second arm. The third activated position was left available for derivatization and linking to various biologically relevant molecules.

The absorption maxima for all species was centered around 340 nm, characteristic of the Lumi4-Tb donor although all species also had a specific absorption peak that was consistent with the direct absorption by the acceptor fluorophore. Emission peaks spanned the visible spectrum from 528 nm-663 nm with extinction coefficients generally in-line with expectations. Quantum yield determinations were 11.2-34.3% yielding high FRET efficiencies ranging from 55% to 92%.

Lumiphore prepared a fluorescein derivative that possessed a long linker (FLX) that was expected to effect the lifetime and efficiency of transfer as proof-of-concept for expanding the dye set in the into the lifetime domain. The ability to offer multiple fluorescein derivatives that emit at a maximal 520 nm, but with different lifetimes, will allow their use together as functionally separate reporters. The long linker did have the expected effect of extended lifetime and reduced quantum yield. A modest lifetime shift from 250 us to 348 us was observed when using an increased linker length. The expected reduction in FRET efficiency and overall quantum yield was observed. Additional investigation of linker constitution and rigidity are required to fully develop this family of reporters and it is expected that other acceptors will have significantly improved performance with similar linking extensions.

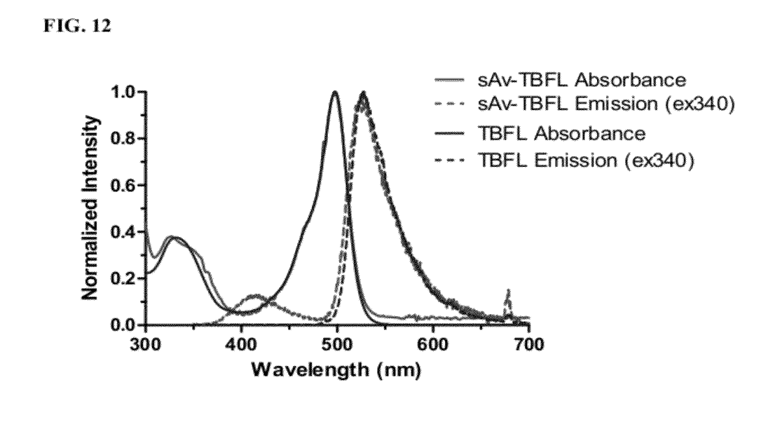

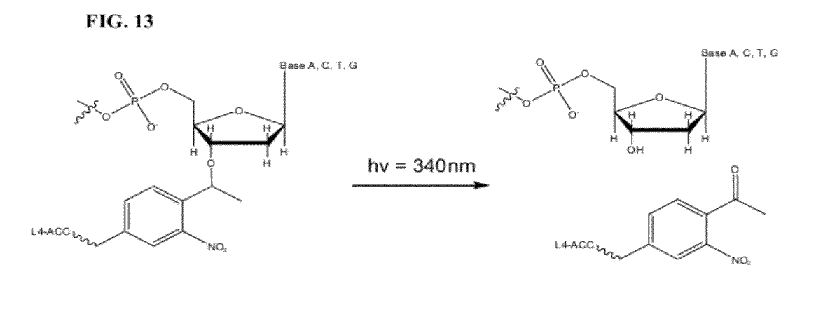

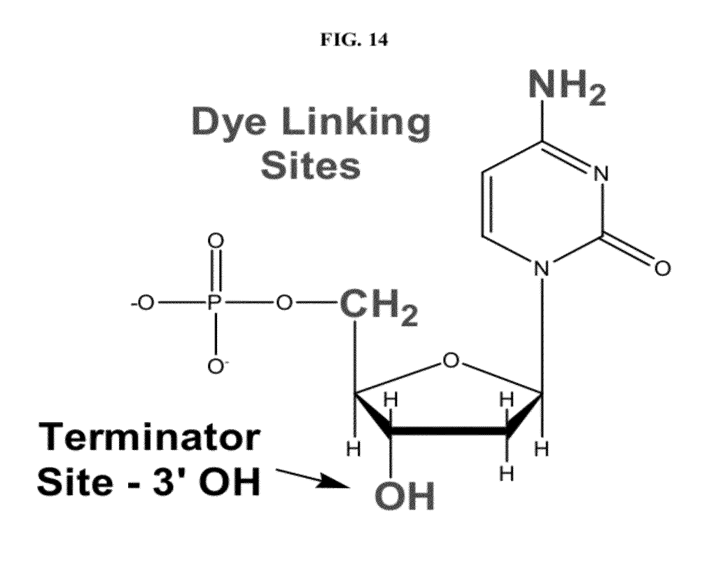

Lumiphore has validated the functionality of the new compounds by coupling with a representative protein—demonstrating that photophysical properties are not significantly altered following conjugation to streptavidin. The fluorescein-Lumi4-Tb construct (TB-FL) was activated in-situ following deprotection with EDC and sulfo-NHS and then coupled to streptavidin under standard coupling conditions. Size exclusion chromatography was used to purify the labeled protein from unbound small molecules. The photophysical properties of the coupled TB-FL was then compared with the free TB-FL (FIG. 12). No appreciable difference in the normalized spectra is observed, with the broad absorption peak at 340 nm characteristic of the Lumi4-Tb compound, the broad absorption peak at 494 nm characteristic of the fluorescein acceptor and the emission peak centered at 520 nm from fluorescein following excitation at 340 nm (normally only excitation at 494 nm would result in a fluorescein emission). This data demonstrates that the compound is robust and is unperturbed by coupled environment of a protein surface.

New molecules are using standard analytical methods such as mass spectrometry, HPLC, elemental analysis, absorption/emission spectra and H¹ and C¹³ NMR as well as photophysical properties such as quantum yield, emission life-time, and extinction coefficient as described below:
Extinction Coefficient. All compounds are purified to a minimum of 95% purity based on mass spectrometry and elemental analysis. A series of solutions of varied concentrations will be prepared from the solids based on mass and purity. The absorbance of each of these solutions at both 340 nm and at the appropriate acceptor wavelength will be measured and plotted according to Beer's Law (A=εCL where A is absorbance, c is the extinction coefficient in M⁻¹ cm⁻¹, C is concentration, and L is path length in cm) with the fitted linear regression slope equal to c on a plot of A as a function of C.
Quantum Yield. Quantum yield is measured using established methods [32, 33]. Fluorescence quantum yield Q are measured in diluted solution with an optical density lower than 0.1 using the following equation Qₓ/Qᵣ=[Aᵣ(λᵣ)/Aₓ(λₓ)][nₓ²/nᵣ²][Dₓ/Dᵣ] were A is the absorbance at the excitation wavelength (λ), n the refractive index and D the integrated luminescence intensity. Subscripts “r” and “x” stand for reference and sample, respectively. References will be a quinine bisulfate in 1N sulfuric acid in aqueous solution (Qᵣ=0.546)¹ and Ru(bpy)₃ 2Cl complexes in water solution (Qᵣ=0.028)² for all complexes. A plot of integrated emission intensity (i.e. Dᵣ) versus absorbance (i.e. Aᵣ(λᵣ)) yields a linear plot with a slope which can be equated to the reference quantum yield Qᵣ. By analogy, for the unknown sample, a plot of integrated emission intensity (i.e. D_{X}) versus absorbance (i.e. Aₓ(λₓ)) yields a linear plot and Qₓ can then be evaluated. A minimum of at least two independent measurements will be averaged to derive a reliable value.
Lifetime Determination. Fluorescent decay signals are acquired using a photon counting module and data analysis performed using the commercially available DAS 6 decay analysis software. Goodness of fit will be assessed by minimizing the reduced chi squared function, χ2, and a visual inspection of the weighted residuals. Each trace will use at least 10,000 points and the reported lifetime values result from at least three independent measurements.

### Preparation of TR-dNTPs

The advantages of using a photocleavable linker as a blocking agent is that no additional chemical reagents are required to be introduced into the system. The 2-nitrobenzyl group is the most widely used photocleavable linking agent and can be modified as a photocleavable linker to bridge functional groups to the nucleotide. It has high photocleavage efficiency under light irradiation at wavelength over 320 nm and does not cause DNA damage. Metzker et al. reported 3′-O-(2-nitrobenzyl)-dA(T)TP may be used as a terminator that can be efficiently removed by UV light in Sanger sequencing. The efficiency of fluorophore removal has been reported to be as high as 80% after UV irradiation at 340 nm. Standard synthetic methods can be used to prepare these compounds in quantities sufficient for further evaluation. Other cleavage strategies exist including catalytic deallylation reactions, enzyme cleavage, disulfide reduction schemes and chemical deactivation reaction that render the fluorophore nn-photoactive.

Additional strategies have been reported in the literature for linking fluorescent compounds via photocleavable linker to positions other than the 3′-hydroxy (Fig.D.4). (Genome Res. 2005, 15, 1767-1776, PNA, 2005, 102, 5926-5931, PNA, 2003, 100, 409-413) Seo et al. demonstrated four distinct fluorescent nucleotide analogs for DNA sequencing. Each different nucleotide analog uses a different fluorophore attached to the 5′ position of the pyrimidines and to the 7′ position of the purines that are linked by a photocleavable 2-nitrobenzyl. All four fluorophores can be detected and cleaved by using near-UV which allows further reading of the DNA sequence. Other options will be investigated if obstacles are encountered, for example, if the relatively large size of the novel reporters is a hinderance to polymerase activity in the 3′ hydroxyl position.

Evaluation of the novel TR reporters on the activity of various DNA polymerase is critical for the advancement of this project as the somewhat greater bulk may have an inhibitory effect due to steric hindrance. This will be evaluated with standard PCR techniques that will utilize the TR-dNTPs in solution with standard dNTPs for thermal cycling of a commercially available DNA plasmid template. Products are evaluated using standard agarose gel electrophoresis. The presence of a ‘ladder or smeared DNA product will indicate that the new reporters will effectively block polymerase activity. A second experiment will utilize the fully cleaved products in solution with the resulting product appearing as a single band at the expected molecular weight.

Photocleavage activity will also be verified via analytical methods such as mass spectrometry, under controlled conditions to rigorously evaluate the efficiency of cleavage and help pinpoint the expected performance in a chip based system with current excitation source energies.

The covalent immobilization of probe sequences onto the surface of an optically useful quartz surface is critical. A number of examples of biomolecule immobilization onto quart surfaces exist in the literature that utilize 5′-amino functionalized oligonucleotides. Oligonucleotides have been anchored to aldehyde-activated glass (Schena, M.; Shalon, D.; Heller, R.; Chai, A.; Brown, P.; Davis, R. Proc. Natl. Acad. Sci. U.S.A. 1996, 93, 10614.), or to glass surfaces modified with epoxide (Lamture, J. B.; Beattie, K. L.; Burke, B. E.; Eggero, H. D.; Ehrlich, D. J.; Fowler, R.; Hollis, M. A.; Kosicki, B. B.; Reich, R. K.; Smith, S. R.; Varma, R. S.; Hogan, M. E. Nucleic Acid Res. 1994, 22, 2121.). Silanized oligonucleotides have also been prepared and immobilized on glass (Kumar, A.; Liang, Z. Nucleic Acid Res. 2001, 29, e2.). An additional approach utilizes Oxanine synthetic base to couple DNA to an amine functionalized glass slide using a one pot synthesis I (Seung Pil Pack, Nagendra Kumar Kamisetty, Mitsuru Nonogawa, Kamakshaiah, Charyulu Devarayapalli, Kaki Ohtani, Kazunari Yamada, Yasuko Yoshida, Tsutomu, Kodaki and Keisuke Makino Nucleic Acids Research, 2007, Vol. 35, No. 17 e110). Our initial strategy will be to leverage commercially available options including thiol terminated oplios (Thermo Electron) and gold coated quartz slides (Nanocs, USA). (Beaucage S. L. et al., (2004) Synthesis of Modified Oligonucleotidesand Conjugates. Current Protocols in Nucleic Acid Chemistry, Chapter 4Bergstrom D. E. et al., (2002) Nucleic Acid based Microarrays and Nanostructures. Current Protocols in Nucleic Acid Chemistry, Chapter 12 Kumar A. et al. (2000) Silanized nucleic acids: a general platform for DNA immobilization. Nucleic Acids Res. 28: e71 Goa, H. et al., (1995))

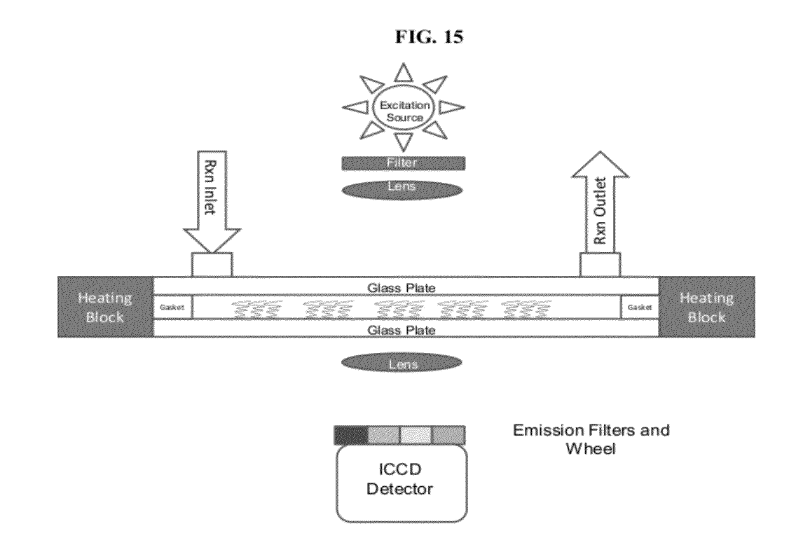

The equipment needed for DNA sequencing utilizing Lumi4®-Tb luminescent tags will consist of commercially available components including: an excitation source, excitation source filter, focusing lens, reaction well, emission focusing lens, emission filters and a detector. The reaction vessel for the DNA template strand extension will be similar to a DNA chip and a flow cell. A general scheme for the device is picture in FIG. 15.

The instrument will be synchronized using a personal computer (PC). A standard TTL trigger pulse that will control the excitation pulse, gating and averaging of the detector, cycling of emission filters, the pumping of the correct reaction solution and washes as need and temperature cycling of the reaction block. The general detection premise is predicated on the Förster resonance energy transfer mechanism (FRET) from the Lumi4-Tb chelate to fluorescent dyes and this will give this system flexibility as many commercially available dyes can potentially be excited by FRET [34-36]. The luminescent tags (Lumi4®-Tb chelate in addition to the fluorescent dyes) tethered to the base can be removed using photocleavage, a process which has previously been described and reported by Ju et al [36-38] in another DNA sequencing approach. The authors concluded that irradiation of the photocleavable linker for 10 sec using a 355 nm pulsed Nd:YAG laser at power of ca. 1.5 W/cm² is sufficient enough to excite and detect their fluorophore labeled nucleotide system, as well as remove the fluorescence tag. This approach will be a core principle of this instrument. We may expand on this excitation/photocleavage photon source strategy to exploit the advantageous time-resolved photoluminescence properties of the Lumi4®-Tb organic fluorophore FRET. We may excite the Lumi4-Tb luminescence agent near the absorbance maxima of 340 nm. The luminescence emission of the covalently linked fluorophores specific to each base will be monitored and used to identify the newly incorporated base.

The luminescent tag would be introduced to the reaction cell by pumping the labeled bases from their respective reservoir with reaction buffer (Thermo Sequenase DNA polymerase, 26 mM TAPS pH of 9.3 and 6.5 mM MgCl₂) using a peristaltic pump system with a multivalve manifold to switch between reaction buffers, nucleic acid solutions and wash [36-38]. After the new base has been ligated by the polymerase to the DNA primer (heating for ˜5 min at 72° C.) the reaction vessels may be washed with a buffered solution (50 mM potassium phosphate pH 6.5, 1 M NaCl 0.1% SDS and 0.1% Tween 20) for ca. 10 min then chamber will be rinsed with deionized water, ethanol and 50:50 acetonitrile/water [38]. The tag and the 3′ capping agent will be excited and cleaved from the nucleobase by irradiation with the excitation source for the appropriate length of time (see Table 1). The next ligation reaction mixture will be introduced to the vessel, followed be the previously described washing regiment to remove the non-ligated labeled nucleobases in the solution, thus removing interfering luminescence in the bulk solution.

Detection of the fluorophore specific bases (such as FITC, Rhodamine 81124, Fluorescein 81009 and Cy5) will be determine by the integrated luminescence signal after a time delay of 100 μs from the excitation pulse and will be averaged over many excitation cycles (ca. 100). Manufacturers and models of potential excitation sources are located in Table 1. Repetition rates, irradiation times, pulse width and/or power can be adjusted to give the largest amount of emission from the nucleic acid labeled fluorophore before the linker is cleaved.

TABLE 1
List of potential excitation sources and manufactures in order of most useful for this apparatus

| | Output | | | | | Pulse | |
|---|---|---|---|---|---|---|---|
| Source | Power | pulse | Wavelength | | | Repetition | Irradiation |
| Type | (W/cm²) | width (ns) | (nm) | Manufacturer | Model | Rate (Hz) | Time (s) |
| Nd:YAG | 1.5 | 4-6 | 355 | Litron Lasers | 150-20 | 10 | 10 |
| Ju et al | 1.5 | 7 | 355 | | | 30 | 10 |
| [37, 38] | | | | | | | |
| Nitrogen | 1.7 | 3.5 | 337.1 | Stanford | NL100 | 0-20 | 18 |
| Laser | | | | Research | | | |
| | | | | Systems | | | |
| Xenon | 60 | 2000-10000 | 275-2000 | Perkin Elmer | FX4401 | up to 1000 | 2 |
| flash lamp | | | | | | | |
| UV-LED | 5 | 10 | 355 | Laser Glow | LQS-355 | 10 | 3 |
| | | | | Technologies | | | |
| Diode | 1.5-0.5 | 10-20 | 355 | CrystaLaser | QUV- | 0-200,000 | 3.5 |
| Pumped | | | | | 355-150 | | |
| Nd:YAG | | | | | | | |

A Xenon flash lamp will provide the greatest flexibility when choosing excitation wavelengths, pulse width, power and irradiation time. However, the long pulse duration (2000 ns) is not ideal for most time-resolved luminescence detection, but should not be an issue with this instrument due to the long fluorescent lifetimes of the novel fluorophores. Monochromatic sources provide a more controlled excitation source with regard to pulse width and excitation wavelength and may not require a filter. However, optical filters may be needed remove unwanted wavelengths from the excitation source and to block stay light from entering the reaction chamber. Depending on the choice of fluorophore reporters selected, emission filters with high transmission will be required to optimize the detection of labeled bases. The multicolored labeling of bases by fluorophores is a detecting scheme currently implemented in commercially sequencing devices [39, 40].

The detectors commonly found in commercial DNA sequencing instruments are charged coupled devices (CCDs), such as the 454 sequencer. Our system can utilize this type of detector; however, our needs require a time-gated detector to remove background luminescence induced by the excitation source such as a gated image-intensified CCD camera (ICDD) from Stanford Research Systems, Princeton Instruments or Hamamatsu with appropriate delay and gate generator [41]. A delay of 100 μs after the excitation will be more than adequate to remove unwanted luminescence. A gate width of 3.0 ms would be appropriate for collection of the dye's emissions. An ideal detector setup would be similar to a digital camera with four (or more) pixels of the CCD detector dedicated to each reaction vessel. Each pixel will have a filter corresponding to the emission wavelength of each base's fluorophore using system similar to the Bayer matrix, thereby allowing detection of each base without using a spectrograph or mechanical moving optics. This approach would be the most cost effective, however, the detection system would suffer from inflexibility (limitation of fluorophore choices and low detector gain). A combination of a sensitive ICCD camera and a mechanical filter wheel controlled by a PC (Apogee AI-AFW25-4R) using three filters (Semrock, Inc.) will be necessary. A filter cycling method optimized to the length at which the luminescent tag resides on the nucleobase will be used to collect three sequential images of the reaction wells and from the intensities the identification of the nucleobase will be determined.

The sequencer plate may be in an array format consisting of multiple patches of template DNA on a glass slide. Each reaction vessel will contain a fragment of the DNA sequence to be analyzed and used as a template for elongation. The DNA template fragments should exist in large copy number (thousands) per patch to give greater signal to noise. The DNA fragments can anchored covalently (or anchored using biotin-streptavidin) to a glass surface of the well to prevent removal by washing [36-39]. The array plate will consist of quartz or optical glass windows to allow the filtered excitation light in and the emitted fluorescent tag luminescence out to the detector. Inlets for either washing the reaction chamber and/or introducing the labeled nucleotides should setup with all patches linked in parallel to the bulk solution of tagged nucleotides. The arrays must be able to withstand temperature changes necessary to drive the elongation of the complimentary DNA strand of the anchored template up to ca. 72° C. [36-40]. An open faced flow cell (CiDRA® Precision Services, LLC.) is an ideal array plate that would meet the needs of this system.

An alternative instrument could be built by modifying a 454 Sequencing unit (Roche Diagnostics Corporation). A pulsed excitation source could be mounted to excite the picotiter plate array and the CCD detector would need to be replaced with a time gated ICCD. Issues that would need to be addressed using this instrumental approach would be excitation pulse and detection timing with the flow sequence. Also excessive scattering and damage to the picotiter plate by the excitation source.

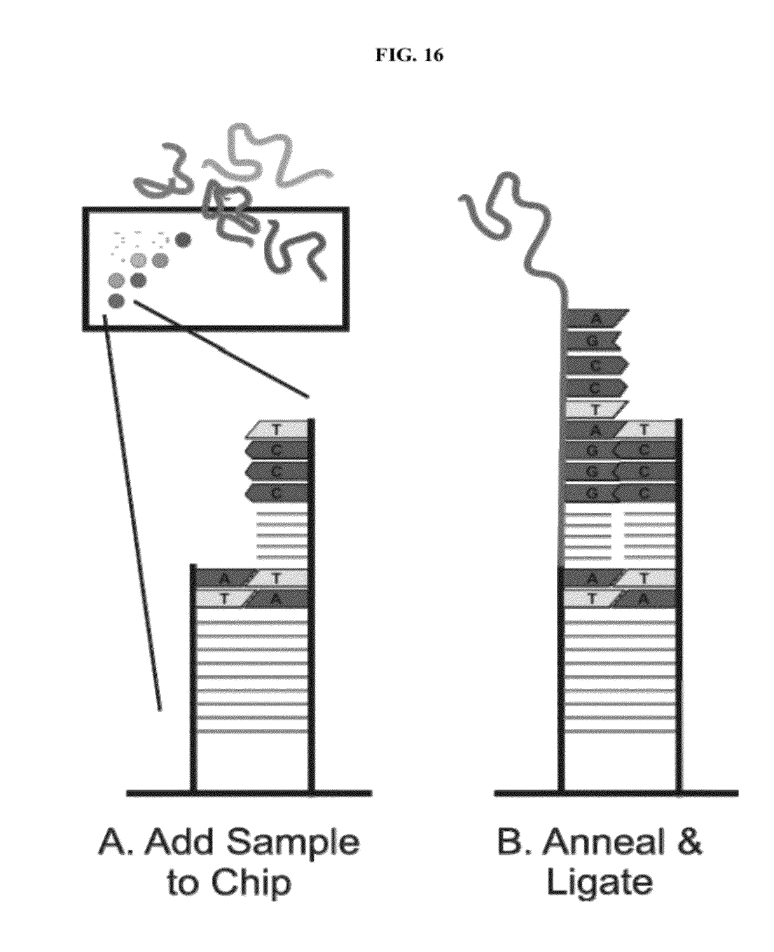

Spot immobilized probe sequences may be incubated with compliment strands that include 10-25 bp of uncomplemented sequence for initial work. These templates will be incubated briefly, annealed and washed with a ligase containing solution to form covalent attachment of the template sequence to the surface anchored probe (FIG. 16). Progressively longer template sequences will be used as the experimental parameters are optimized, but sequence reads well beyond 500 by are expected.

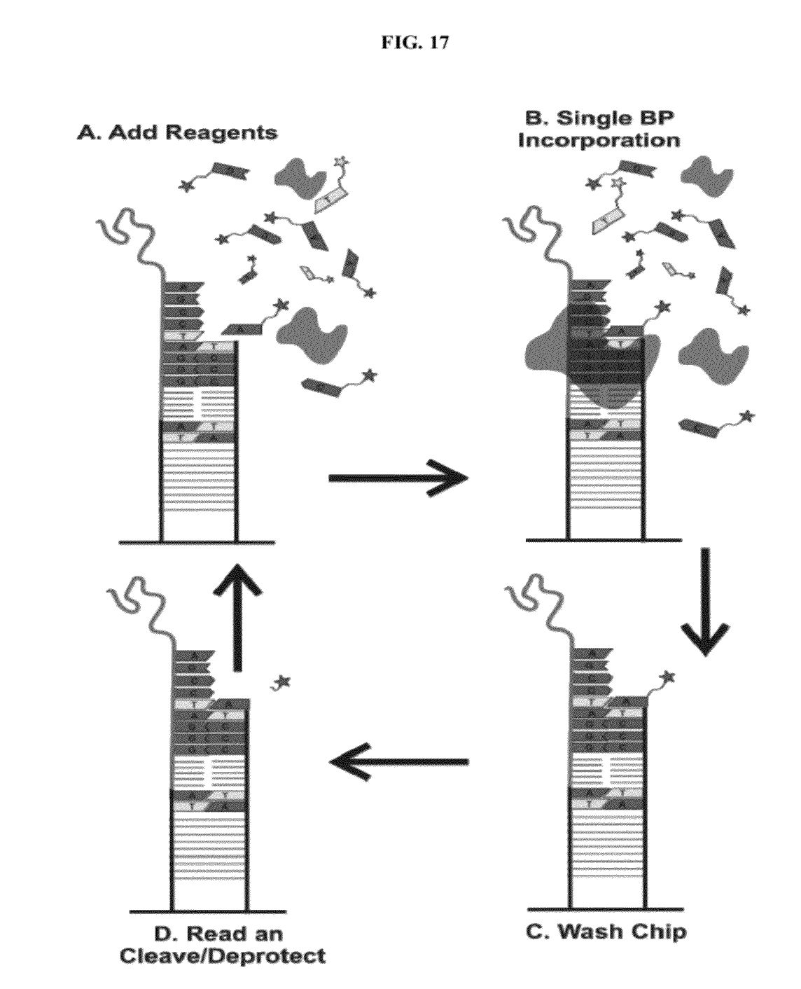

Sequencing by synthesis (SBS) will occur in a cycled fashion alternating base incorporation, polymerase bond formation, washing and read/cleavage steps (FIG. 17). During the initial step, the previously prepared chip will be flushed with a solution containing requisite reagents for polymerase catalysed TR-dNTP incorporation. This step may be controlled with microfluidic controllers from a temperature controlled reservoir that will allow recycling of reagents to reduce cost. Efforts will also be made to reduce flow-through and waste volumes to manage reagent costs. Previous experiments will have demonstrated that the fluorophore linking site is non-disruptive to the polymerase activity allowing base incorporation. Previous experiments will also have identified optimal polymerases for our substrates that combine fidelity and robustness. Since the proposed system is not real-time (relies on polymerase gating) the speed of the polymerase activity is the least important factor. The third step is a washing step where stringent conditions will remove all free reporters and polymerase from the template strand and offer an optically optimized solution for excitation, emission and blocker cleavage. Typically, the Lumi4-Tb reporter has no photobleaching susceptibility and thus repeated flash/read cycles are permitted to improve signal to noise ratios. In the current proposal, this repeated flashing will have the effect of continual erosion of the novel reporter into solution as it is cleaved; however, this will not degrade its fluorescence intensity and will remain as a signal generator until excitation ends. The critical aspect of the number of excitation pulses will be the necessity for nearly 100% effective cleavage. Incomplete cleavage in any cycle will increasingly convolute and degrade subsequent base pair reads as sequence strands will be out of sync an varied reported emissions will be intertwined. Following cleavage, the buffer is washed with an injection of the initial reaction buffer for the next base pair incorporation.

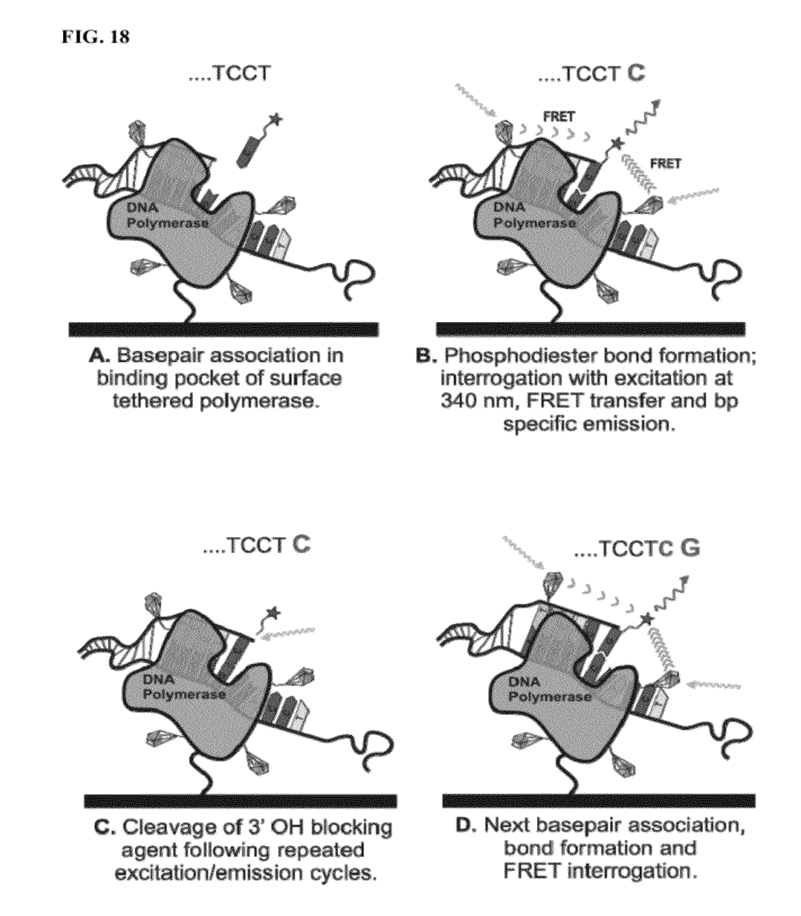

An alternate approach that will be investigated relies heavily on various acceptor fluorophores but separates the donor and acceptor molecules in a more traditional FRET approach (FIG. 18). In this approach, the polymerase is labeled with the Lumi4-Tb donor and is immobilized onto a quartz substrate from a dilute enough solution to allow optical isolation of individual enzymes. Conventionally labeled and photocleavable dNTPs are allowed to react with strand template at each immobilized positions and the cycling is followed by CCD equipment. This approach has the primary advantage that no washing is required as signal is generated only at the proximal interface of the polymerase and the incorporating dNTPs. Sequencing can continue at the rate of incorporation and is limited by the efficiency of cleavage. Some rate control may be implemented via temperature regulation may be required due to the long lifetime of the donor-acceptor pair.

This approach is only viable due to the exceptionally high quantum yield of Lumi4-Tb as well as the high transfer efficiencies observed in studies. In one mode, dNTP's would be labeled at a site that did not significantly hinder polymerase activity and the synthesis would be monitored in real time. As the synthesized strand moved out of the binding pocket the signal intensity from already incorporated bases would diminish. Corrective strategies could be used to deconvolute any overlapping signals from adjacent bases. A second approach utilizes the photocleavable acceptors and requires sufficient excitation pulses to cleave the blocking fluorophore before the next incorporation event. Both strategies have significant hurdles to overcome, however, recent successes following real-time polymerase activity, coupled with the significant advantages of TR-FRET make this approach, if successful, a real paradigm shift in next generation sequencing.

The articles “a,” “an” and “the” as used herein do not exclude a plural number of the referent, unless context clearly dictates otherwise. The conjunction “or” is not mutually exclusive, unless context clearly dictates otherwise. The term “include” is used to refer to non-exhaustive examples.

All references, publications, patent applications, issued patents, accession records and databases cited herein, including in any appendices, are incorporated by reference in their entirety for all purposes.

### CITATIONS

1. Petoud, S., et al., Stable lanthanide luminescence agents highly emissive in aqueous solution: multidentate 2-hydroxyisophthalamide complexes of Sm(3+), Eu(3+), Tb(3+), Dy(3+). J Am Chem Soc, 2003. 125(44): p. 13324-5.
2. Mathis, G., HTRF(R) Technology. J Biomol Screen, 1999. 4(6): p. 309-314.
3. Beeby, A., et al., Luminescence imaging microscopy and lifetime mapping using kinetically stable lanthanide(III) complexes. J Photochem Photobiol B, 2000. 57(2-3): p. 83-9.
4. Vereb, G., et al., Temporally and spectrally resolved imaging microscopy of lanthanide chelates. Biophys J, 1998. 74(5): p. 2210-22.
5. Glazer, A. N., Light harvesting by phycobilisomes. Annu Rev Biophys Biophys Chem, 1985. 14: p. 47-77.
6. Batard, P., et al., Use of phycoerythrin and allophycocyanin for fluorescence resonance energy transfer analyzed by flow cytometry: advantages and limitations. Cytometry, 2002. 48(2): p. 97-105.
7. Bruchez, M., Jr., et al., Semiconductor nanocrystals as fluorescent biological labels. Science, 1998. 281(5385): p. 2013-6.
8. Chan, W. C. and S, Nie, Quantum dot bioconjugates for ultrasensitive nonisotopic detection. Science, 1998. 281(5385): p. 2016-8.
9. Mattoussi, H., et al., Self-assembly of CdSe—ZnS quantum dot bioconjugates using an engineered recombinant protein. Journal of the American Chemical Society, 2000. 122(49): p. 12142-12150.
10. Tsien, R. Y., The green fluorescent protein. Annu Rev Biochem, 1998. 67: p. 509-44.
11. Wang, L., et al., Unnatural amino acid mutagenesis of green fluorescent protein. J. Org. Chem., 2003. 68(1): p. 174-6.
12. Smith, L. M., et al., Fluorescence detection in automated DNA sequence analysis. Nature, 1986. 321(6071): p. 674-9.
13. Prober, J. M., et al., A system for rapid DNA sequencing with fluorescent chain-terminating dideoxynucleotides. Science, 1987. 238(4825): p. 336-41.
14. Collins, F. S., et al., A vision for the future of genomics research. Nature, 2003. 422(6934): p. 835-47.
15. Ju, J., et al., Fluorescence energy transfer dye-labeled primers for DNA sequencing and analysis. Proc Natl Acad Sci USA, 1995. 92(10): p. 4347-51.
16. Metzker, M. L., J. Lu, and R. A. Gibbs, Electrophoretically uniform fluorescent dyes for automated DNA sequencing. Science, 1996. 271(5254): p. 1420-2.
17. Lee, L. G., et al., New energy transfer dyes for DNA sequencing. Nucleic Acids Res, 1997. 25(14): p. 2816-22.
18. Brumley, R. L., Jr. and L. M. Smith, Rapid DNA sequencing by horizontal ultrathin gel electrophoresis. Nucleic Acids Res, 1991. 19(15): p. 4121-6.
19. Kheterpal, I., et al., DNA sequencing using a four-color confocal fluorescence capillary array scanner. Electrophoresis, 1996. 17(12): p. 1852-9.
20. Metzker, M. L., Emerging technologies in DNA sequencing. Genome Res, 2005. 15(12): p. 1767-76.
21. Nunnally, B. K., et al., Characterization of visible dyes for four-decay fluorescence detection in DNA sequencing. Anal Chem, 1997. 69(13): p. 2392-7.
22. Lieberwirth, U., et al., Multiplex dye DNA sequencing in capillary gel electrophoresis by diode laser-based time-resolved fluorescence detection. Anal Chem, 1998. 70(22): p. 4771-9.
23. Lassiter, S. J., et al., Time-resolved fluorescence imaging of slab gels for lifetime base-calling in DNA sequencing applications. Anal Chem, 2000. 72(21): p. 5373-82.
24. Alayerdian, L., et al., A family of novel DNA sequencing instruments based on single photon detection. Electrophoresis, 2002. 23(16): p. 2804-17.
25. Kling, J., Ultrafast DNA sequencing. Nat Biotechnol, 2003. 21(12): p. 1425-7.
26. Bünzli, J. C. G. and G. R. Choppin, Lanthanide probes in life, chemical, and earth sciences: theory and practice. 1989, Amsterdam; New York: Elsevier. xiii, 432.
27. Brunet, E., et al., Lanthanide complexes of polycarboxylate-bearing dipyrazolylpyridine ligands with near-unity luminescence quantum yields: the effect of pyridine substitution. Photochem Photobiol Sci, 2002. 1(8): p. 613-8.
28. Alpha, B., et al., Quantitative photophysical results of luminescence studies: Eu3+ and Tb3+ cryptates of macrobicyclic polypyridine ligands. Angewandte Chemie, International Edition, 1987. 26: p. 1266.
29. Hemmila, I., Luminescent Lanthanide Chelates—a Way to More Sensitive Diagnostic Methods. Journal of Alloys and Compounds, 1995. 225(1-2): p. 480-485.
30. Xiao, M. and P. R. Selvin, Quantum yields of luminescent lanthanide chelates and far-red dyes measured by resonance energy transfer. J Am Chem Soc, 2001. 123(29): p. 7067-73.
31. Ullman, E. F., M. Schwarzberg, and K. E. Rubenstein, Fluorescent excitation transfer immunoassay. A general method for determination of antigens. J Biol Chem, 1976. 251(14): p. 4172-8.
32. Demas, J. N. and G. A. Crosby, The measurement of photoluminescence quantum yields. Journal Phys. Chem., 1971. 75: p. 991-1024.
33. Mugabe, C., A. Azghani, and A. Omri, Liposome-mediated gentamicin delivery: development and activity against resistant strains of Pseudomonas aeruginosa isolated from cystic fibrosis patients. J Antimicrob Chemother, 2005. 55(2): p. 269-271.
34. Förster, T., Ann. Phys., 1948. 2: p. 55.
35. Förster, T., Faraday Soc., 1959. 27: p. 7-17.
36. Ruparel, H., et al., Proceedings of the National Academy of Sciences of the United States of America, 2005. 102: p. 5932-5937.
37. Li, Z., et al., A photocleavable fluorescent nucleotide for DNA sequencing and analysis. Proceedings of the National Academy of Sciences of the United States of America, 2003. 100: p. 414-419.
38. Seo, T. S., et al., Four-color DNA sequencing by synthesis on a chip using photocleavable fluorescent nucleotides. Proceedings of the National Academy of Sciences of the United States of America, 2005. 102: p. 5926-5931.
39. Eid, J., et al., Real-Time DNA Sequencing from Single Polymerase Molecules. Science, 2009. 323(5910): p. 133-138.
40. Margulies, M., et al., Genome sequencing in microfabricated high-density picolitre reactors. Nature, 2005. 437(7057): p. 376-380.
41. Rajapakse, H. E., et al., Luminescent Terbium Protein Labels for Time-Resolved Microscopy and Screening. Angew. Chem. Int. Ed., 2010. 48: p. 4990-4992.

We claim:

1. A method of detecting a nucleotide, the method comprising:
(a) forming a complex between a nucleic acid and a DNA polymerase comprising a first luminescent group, wherein the nucleic acid comprises a template strand and a primer hybridized to the template strand;
(b) extending the primer with the nucleotide by contacting the complex with the nucleotide, wherein the nucleotide is a dNTP comprising a second luminescent group;
(c) exciting the complex with light, whereby energy is transferred between the first luminescent group and the second luminescent group; and
(d) detecting energy emitted by the complex, thereby detecting the nucleotide.

2. The method of claim 1 wherein the DNA polymerase is bound to a solid support.

3. The method of any preceding claim wherein the first luminescent group is an energy transfer donor.

4. The method of claim 3 wherein the energy transfer donor has a structure according to the formula: 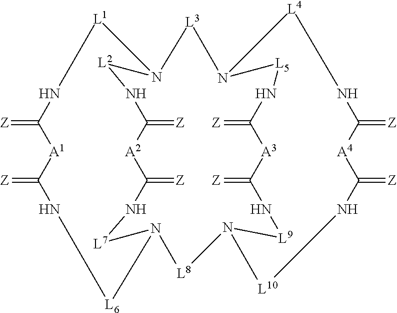
wherein each Z is a member independently selected from O and S;
L¹, L², L³, L⁴, L⁵, L⁶, L⁷, L⁸, L⁹ and L¹⁰ are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl;
A¹, A², A³ and A⁴ are members independently selected from the general structure: 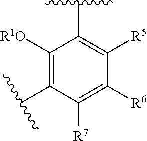
wherein each R¹ is a member independently selected from H, an enzymatically cleavable group, a hydrolytically cleavable group, a metabolically cleavable group and a single negative charge; and
each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and NO₂,
wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl; and
R¹⁷ and R¹⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R¹⁷ and R¹⁸, together with the atoms to which they are attached, are optionally joined to form a 5- to 7-membered ring,
wherein the energy transfer donor comprises a linkage to the DNA polymerase.

5. The method of claim 4, wherein the energy transfer donor has the structure: 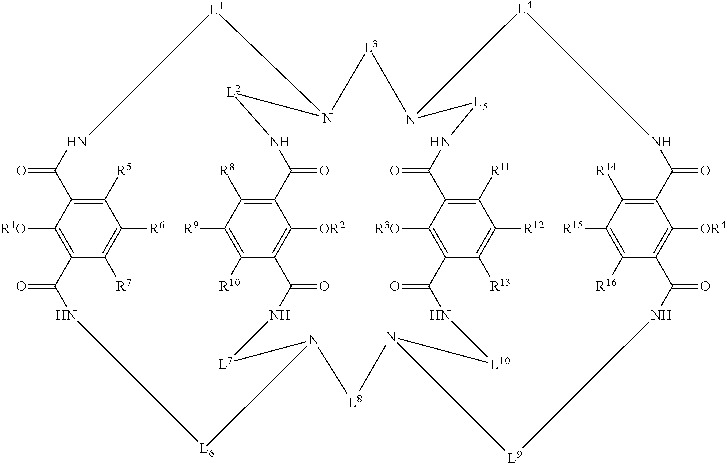
wherein R², R³ and R⁴ have the same definition as R¹; and
R⁸, R⁹ and R¹⁰ have the same definition as R⁵, R⁶ and R⁷, respectively; R¹¹, R¹² and R¹³ have the same definition as R⁵, R⁶ and R⁷, respectively; and R¹⁴, R¹⁵ and R¹⁶ have the same definition as R⁵, R⁶ and R⁷, respectively.

6. The method of claim 5, wherein the energy transfer donor has a structure selected from: 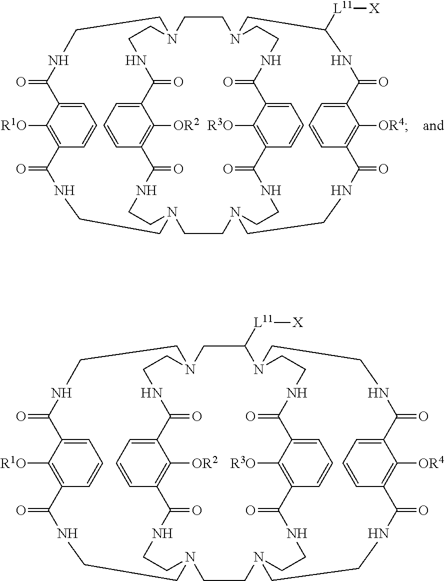
wherein L¹¹ is a member selected from a bond, acyl, substituted or unsubstituted alkyl, substituted or unsubstituted heteroalkyl, substituted or unsubstituted aryl, substituted or unsubstituted heteroaryl, substituted or unsubstituted heterocycloalkyl and nucleic acid; and
X is a linkage fragment covalently binding the DNA polymerase to L¹¹.

7. The method of any of claims 4 and 5, wherein the linker moieties L¹, L², L³, L⁴, L⁵, L⁶, L⁷, L⁸, L⁹ and L¹⁰ are members independently selected from substituted or unsubstituted C₁ to C₆ alkyl.

8. The method of any of claims 4, 5 and 7, wherein the linker moieties L¹, L², L³, L⁴, L⁵, L⁶, L⁷, L⁸, L⁹ and L¹⁰ are members independently selected from substituted or unsubstituted ethyl.

9. The method of any of claims 4, 5, 7 and 8 wherein at least one of L¹, L², L³, L⁴, L⁵, L⁶, L⁷, L⁸, L⁹ and L¹⁰ comprises a linkage to the DNA polymerase.

10. The method of any preceding claim wherein the dNTP is not blocked at the 3′ position.

11. The method of any of claims 1-9 wherein the dNTP is blocked at the 3′ position.

12. The method of claim 11 wherein the dNTP is covalently linked at the 3′ position to a cleavable group.

13. The method of claim 12 wherein the cleavable group is selected from a hydrolytically cleavable group, an enzymatically cleavable group and a photolytically cleavable group.

14. The method of claim 13 wherein the cleavable group is a photolytically cleavable group.

15. The method of any of claims 13 and 14 wherein the photolytically cleavable group comprises 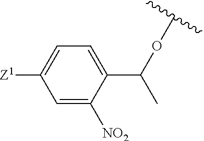
wherein Z¹ is the second luminescent group.

16. The method of any of claims 12-15 comprising exposing the complex to UV light.

17. The method of any preceding claim wherein the second luminescent group is a fluorophore acceptor.

18. The method of any preceding claim wherein one of the luminescent groups is chelated to a metal ion.

19. The method of claim 18 wherein the metal ion is a lanthanide ion.

20. The method of claim 19, wherein the lanthanide is a selected from neodynium (Nd), samarium (Sm), europium (Eu), terbium (Tb), dysprosium (Dy) and ytterbium (Yb).

21. A method of detecting a nucleotide, the method comprising:
(a) forming a complex between a nucleic acid and a capture probe, wherein the capture probe is bound to a solid support;
(b) contacting the complex with a DNA polymerase and the nucleotide, wherein the nucleotide is a blocked dNTP comprising a first luminescent group and a second luminescent group, thereby extending the nucleic acid with the nucleotide;
(c) washing the solid support;
(d) exciting the complex with light, whereby energy is transferred between the first luminescent group and the second luminescent group; and
(e) detecting energy emitted by the second luminescent group, thereby detecting the nucleotide.

22. The method of claim 21 wherein the first luminescent group is an energy transfer donor, the second luminescent group is an energy transfer acceptor, the donor and acceptor are covalently joined to form a donor-acceptor assembly, and the donor-acceptor assembly is joined to a dNTP by a donor-acceptor linker comprising a cleavable group.

23. The method of claim 21 wherein the first luminescent group is an energy transfer donor, the second luminescent group is an energy transfer acceptor, and the donor and acceptor are joined by a donor-acceptor linker comprising a cleavable group.

24. The method of any of claims 22 and 23 wherein the energy transfer donor has a structure according to the formula: 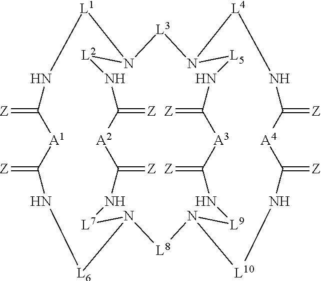
wherein each Z is a member independently selected from O and S;
L¹, L², L³, L⁴, L⁵, L⁶, L⁷, L⁸, L⁹ and L¹⁰ are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl;
A¹, A², A³ and A⁴ are members independently selected from the general structure: 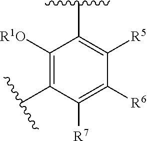
wherein each R¹ is a member independently selected from H, an enzymatically cleavable group, a hydrolytically cleavable group, a metabolically cleavable group and a single negative charge; and
each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and NO₂,
wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl; and
R¹⁷ and R¹⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R¹⁷ and R¹⁸, together with the atoms to which they are attached, are optionally joined to form a 5- to 7-membered ring,
wherein the energy transfer donor comprises a linkage to the donor-acceptor linker.

25. The method of claim 24 wherein the energy transfer donor has the structure: 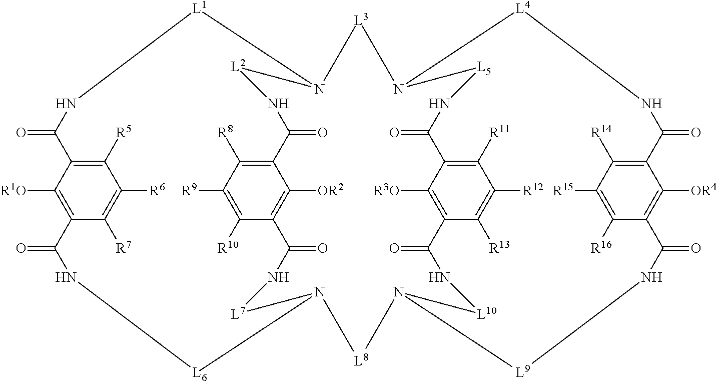
wherein R², R³ and R⁴ have the same definition as R¹; and
R⁸, R⁹ and R¹⁰ have the same definition as R⁵, R⁶ and R⁷, respectively; R¹¹, R¹² and R¹³ have the same definition as R⁵, R⁶ and R⁷, respectively; and R¹⁴, R¹⁵ and R¹⁶ have the same definition as R⁵, R⁶ and R⁷, respectively.

26. The method of claim 25, wherein the energy transfer donor has a structure selected from: 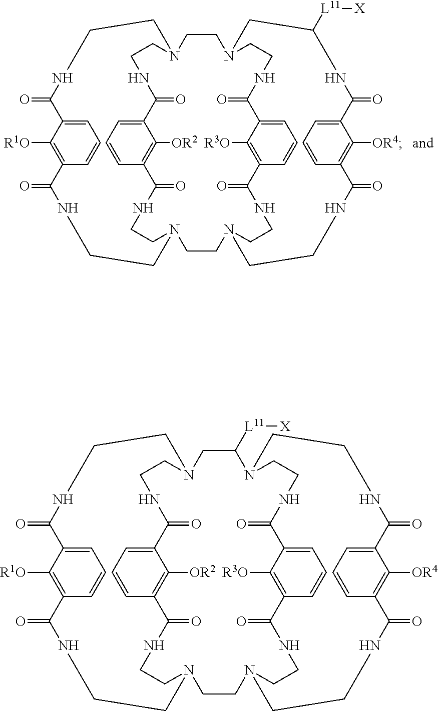
wherein L¹¹ is a member selected from a bond, acyl, substituted or unsubstituted alkyl, substituted or unsubstituted heteroalkyl, substituted or unsubstituted aryl, substituted or unsubstituted heteroaryl, substituted or unsubstituted heterocycloalkyl and nucleic acid; and
X is a linkage fragment covalently binding the donor-acceptor linker to L¹¹.

27. The method of any of claims 24 and 25, wherein the linker moieties L¹, L², L³, L⁴, L⁵, L⁶, L⁷, L⁸, L⁹ and L¹⁰ are members independently selected from substituted or unsubstituted C₁ to C₆ alkyl.

28. The method of any of claims 24, 25 and 27, wherein the linker moieties L¹, L², L³, L⁴, L⁵, L⁶, L⁷, L⁸, L⁹ and L¹⁰ are members independently selected from substituted or unsubstituted ethyl.

29. The method of any of claims 24, 25, 27 and 28 wherein at least one of L¹, L², L³, L⁴, L⁵, L⁶, L⁷, L⁸, L⁹ and L¹⁰ comprises a linkage to the linker between the first luminescent group and the second luminescent group.

30. The method of any of claims 22-29 wherein the donor-acceptor linker comprises substituted or unsubstituted aryl or substituted or unsubstituted heteroaryl.

31. The method claim 30 wherein the donor-acceptor linker comprises 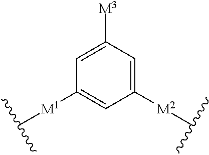
wherein M¹ and M² are independently selected from substituted or unsubstituted alkyl and substituted or unsubstituted heteroalkyl; and M³ is a linker to DNA and comprises a cleavable group.

32. The method of any of claims 22-31 wherein the cleavable group is selected from a hydrolytically cleavable group, an enzymatically cleavable group and a photolytically cleavable group.

33. The method of any of claims 22-32 wherein the cleavable group comprises

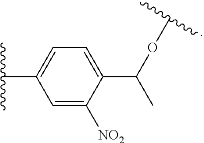

34. The method of any of claims 22-33 wherein the energy transfer acceptor is a fluorophore acceptor.

35. The method of any preceding claim wherein one of the luminescent groups is chelated to a metal ion.

36. The method of claim 35 wherein the metal ion is a lanthanide ion.

37. The method of claim 36, wherein the lanthanide is a selected from neodynium (Nd), samarium (Sm), europium (Eu), terbium (Tb), dysprosium (Dy) and ytterbium (Yb).

38. A compound having the structure:
Q¹-G
wherein Q¹ is a luminescent group and G is a cleavable group.

39. The compound of claim 38 wherein Q¹ is an energy transfer acceptor.

40. The compound of claim 39 wherein Q¹ is a fluorophore acceptor.

41. The compound of any of claims 38-40 wherein G is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

42. The compound of claim 41 wherein G is substituted or unsubstituted aryl.

43. The compound of claim 42 wherein G comprises 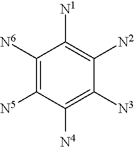
wherein N¹, N², N³, N⁴, N⁵ and N⁶ are independently selected from H, halogen, haloalkyl, —NO₂, —CN, —SO₃H, —CO₂H, —CHO, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and
one of N¹, N², N³, N⁴, N⁵ and N⁶ is bonded to Q¹.

44. The compound of any of claims 38-43 having the structure 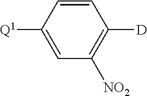
wherein D is selected from —C(O)CH₃, —C(O)(0)CH₃ and a linkage to a nucleotide.

45. A compound having the structure:
Q²-G
wherein Q² comprises a first luminescent group and a second luminescent group; and G is a cleavable group; wherein the first luminescent group is an energy transfer donor and the second luminescent group is an energy transfer acceptor.

46. The compound of claim 45 wherein G is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

47. The compound of claim 46 wherein G is substituted or unsubstituted aryl.

48. The compound of claim 47 wherein G comprises 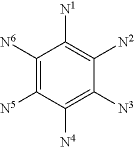
wherein N¹, N², N³, N⁴, N⁵ and N⁶ are independently selected from H, halogen, haloalkyl, —NO₂, —CN, —SO₃H, —CO₂H, —CHO, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and
one of N¹, N², N³, N⁴, N⁵ and N⁶ is bonded to Q².

49. The compound of any of claims 45-48 having the structure 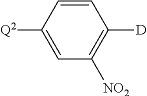
wherein D is selected from —C(O)CH₃, —C(O)(O)CH₃ and a linkage to a nucleotide.

50. The compound of any of claims 45-49 wherein the energy transfer acceptor is a fluorophore acceptor.

51. The compound of any of claims 45-50 wherein the energy transfer donor has a structure according to the formula: 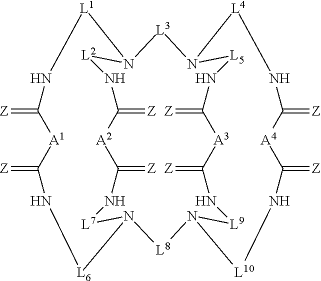
wherein each Z is a member independently selected from O and S;
L¹, L², L³, L⁴, L⁵, L⁶, L⁷, L⁸, L⁹ and L¹⁰ are linker groups independently selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl;
A¹, A², A³ and A⁴ are members independently selected from the general structure: 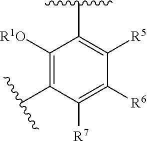
wherein each R¹ is a member independently selected from H, an enzymatically cleavable group, a hydrolytically cleavable group, a metabolically cleavable group and a single negative charge; and
each R⁵, R⁶ and R⁷ is a member independently selected from H, substituted or unsubstituted alkyl, substituted or unsubstituted heteroalkyl, halogen, CN, CF₃, acyl, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and NO₂,
wherein R⁶ and a member selected from R⁵, R⁷ and combinations thereof are optionally joined to form a ring system which is a member selected from substituted or unsubstituted cycloalkyl, substituted or unsubstituted heterocycloalkyl, substituted or unsubstituted aryl and substituted or unsubstituted heteroaryl; and
R¹⁷ and R¹⁸ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R¹⁷ and R¹⁸, together with the atoms to which they are attached, are optionally joined to form a 5- to 7-membered ring.

52. The compound of any of claims 45-51 wherein Q² has the structure 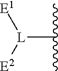
wherein E¹ is the energy transfer donor, E² is the energy transfer acceptor and L is a linker selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

53. The compound of claim 52 wherein Q² has the structure 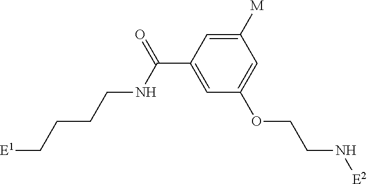
wherein M is a linkage to G.

54. The compound of any of claims 38-53 wherein one of the luminescent groups is chelated to a metal ion.

55. The compound of claim 54 wherein the metal ion is a lanthanide ion.

56. The compound of claim 55, wherein the lanthanide is a selected from neodynium (Nd), samarium (Sm), europium (Eu), terbium (Tb), dysprosium (Dy) and ytterbium (Yb).

57. The compound of any of claims 39-56 wherein the energy transfer acceptor is a compound disclosed herein.

58. The compound of any of claims 45-56 wherein the energy transfer donor is a compound disclosed herein.

59. The method of any of claims 1-37 wherein the first luminescent group or the second luminescent group is a compound disclosed herein.
